(19) United States
(12) Patent Application Publication
(10) Pub. No.: US 2024/0366135 A1
(43) Pub. Date: Nov. 7, 2024

(54) BINDER-FREE STRETCHABLE INTERCONNECT

(71) Applicant: Nanyang Technological University, Singapore (SG)

(72) Inventors: Ying JIANG, Singapore (SG); Zhiyuan LIU, Singapore (SG); Xiaodong CHEN, Singapore (SG)

(21) Appl. No.: 18/686,080

(22) PCT Filed: Aug. 25, 2022
(86) PCT No.: PCT/SG2022/050607

§ 371 Date: Feb. 23, 2024

(30) Foreign Application Priority Data
Aug. 26, 2021 (SG) 10202109356X
Nov. 29, 2021 (SG) 10202113264R

(51) Int. Cl. A61B 5/268; A61B 5/265; A61B 5/296; A61N 1/05

(52) U.S. Cl. CPC A61B 5/268; A61B 5/265; A61B 5/296; A61N 1/0551; A61B 2562/125; A61B 2562/164

(57) ABSTRACT

Herein disclosed include a flexible electronic device comprising a first component comprising a first biphasic portion, a second component, wherein the first component and the second component are in contact with an electrically conductive stretchable interface configured between the first component and the second component, wherein the electrically conductive stretchable interface comprises the first biphasic portion which is adhered to a portion of the second component, and wherein the first biphasic portion comprises a first polymer having (i) a surface partially covered with metal nanoparticles which are partially exposed at the surface, and (ii) metal nanoparticles which are completely embedded in the first polymer. The disclosure also includes a method of forming the flexible electronic device.

### CROSS-REFERENCE TO RELATED APPLICATION

This application claims the benefit of priority of Singapore Patent Application No. 10202109356X, filed on 26 Aug. 2021, and Singapore Patent Application No. 10202113264R, filed on 29 Nov. 2021, the content of it being hereby incorporated by reference in its entirety for all purposes.

### TECHNICAL FIELD

The present disclosure relates to a flexible electronic device and its uses. The present disclosure also relates to a method of forming the flexible electronic device.

### BACKGROUND

Stretchable hybrid electronics may have attracted tremendous attention for implantable/on-skin healthcare applications in recent decades, owing to its high endurance against mechanical deformation. Traditionally, stretchable hybrid electronics may consist of three types of elementary components: protective encapsulation with selective exposure, functional soft component with mechanical matching of human/soft robotics, and rigid component with silicon-based (Si-based) microelectronics technology (FIG. 1A). These components may be independently manufactured because of their disparate processing techniques, functions, form factor, material selections, before being integrated together through modular interfaces. Said differently, stretchable hybrid devices may be assembled by connecting different modules together (FIG. 1A). The terms “components” and “modules” may be interchangeably used in the present disclosure. The multifarious modules may be classified as three elementary types: soft modules that mechanically matches tissue and/or skin of humans or soft robots, rigid modules comprising the Si-based microelectronics, and encapsulation modules for protection.

Although the elementary components (or modules) may have been extensively explored, the interfaces between such elementary components still suffer from weak bonding and low stretchability, which undesirably limits the robustness and complexity of the whole device. This may be because every module tends to be made of different materials, have different form factors, and require disparate processing techniques, manufactured independently and assembled thereafter using traditionally available conductive pastes such as anisotropic conductive film (ACF) and silver paste. For instance, a traditional common approach for the connection may be to use available conductive paste, e.g. anisotropic conductive film (ACF), silver paste, and copper tape. Unfortunately, the pastes tend to introduce mechanical mismatch and weak bonding with soft components, leading to interfacial failure under mechanical deformation (FIG. 1G to 1N). Other traditionally proposed methods may include developing an all-soft electronics (without rigid silicon (Si) component) to eliminate the interface, using liquid metal to avoid rigid paste (but the surface tension of the liquid metal tends to provide undesirably low mechanical adhesion and hence smears easily), and/or using self-healing conductive composite as paste substitute (used due to their voluminous bulk phase, rather than interfacial phenomena, leading to mechanical mismatch and inapplicability for ultrahin electronics). However, for the all-soft electronics approach there may still be a huge gap in matching the performance of Si-based electronics, for the liquid metal it may easily leak when in direct contact with human skin/organ, and for the conductive self-healing composite approach the bonding capability results from its bulky volume rather than surface which tends to be difficult to pattern and hence unable to fulfill applications that require an ultrathin thickness.

There is thus a need to provide for a solution that addresses one or more of the limitations mentioned above.

### SUMMARY

Various non-limiting embodiments relate to a universal interface that can reliably connect soft, rigid and encapsulation modules together, in a plug-and-play manner, to form robust and highly stretchable devices. The universal interface can be referred in the present disclosure as a “biphasic nano-dispersed (BIND) interface”.

In various non-limiting embodiments, the interface may include interpenetrating phases of metallic nanoparticles and soft elastomeric polymer, which may connect any of aforesaid modules by simply pressing together without using pastes. Soft-soft modules joined by this interface achieved 600% and 180% mechanical and electrical stretchability, respectively. Soft modules and rigid modules based on, for example, polyimide, polyethylene terephthalate and silicon substrates can also be connected. Encapsulation on soft modules is strongly adhesive, displaying interfacial toughness up to 0.24 N/mm, 60 times larger than traditional encapsulation. Any module bearing the BIND interface can simply be pressed together face-to-face to form the BIND connection(s) in a short time (FIG. 1C). The BIND interface can be prepared, for example, by thermally evaporating gold (Au) or silver (Ag) nanoparticles onto a ˜100 μm thick self-adhesive styrene-ethylene-butylene-styrene (SEBS) thermoplastic elastomer, which represents a soft module widely used in stretchable electronics (see, for instance, example 2A). Nanoparticles near the surface of the SEBS matrix can form a biphasic structure, wherein some are immersed in the SEBS matrix while others are partly exposed above the matrix (FIG. 1B). This interfacial structure produces exposed SEBS and Au on the surface and interpenetrating Au nanoparticles inside the matrix. Exposed SEBS enhances binding while exposed and immersed Au nanoparticles maintain a continuous electrical pathway, allowing modules with BIND connections to remain functional even when stretched or deformed.

Certain non-limiting embodiments relate to a straightforward stretchable electrode for use in in vivo neuromodulation involving aforesaid interface. Certain non-limiting embodiments also relate to a more complex on-skin electromyography electrode involving aforesaid interface. The modular integration improves signal quality and electrode performance in all embodiments. It can be expected that such an interface, which is workable as a plug-and-play interface, simplifies and accelerates the development of on-skin and implantable stretchable devices.

Traditionally, stretchable hybrid devices have enabled high-fidelity implantable and on-skin monitoring of physiological signals. These traditional devices may contain soft modules that match the mechanical requirements in humans and soft robots, rigid modules containing Si-based microelectronics and protective encapsulation modules. While these devices may be mechanically compliant, the connection (i.e. interface) between the modules may experience stress concentration that limit their stretching and ultimately cause debonding failure. The interface as mentioned above is able to address such limitations.

In a first aspect, there is provided for a flexible electronic device comprising:
a first component comprising a first biphasic portion;
a second component,
wherein the first component and the second component are in contact with an electrically conductive stretchable interface configured between the first component and the second component,
wherein the electrically conductive stretchable interface comprises the first biphasic portion which is adhered to a portion of the second component, and
wherein the first biphasic portion comprises a first polymer having
   (i) a surface partially covered with metal nanoparticles which are partially exposed at the surface, and
   (ii) metal nanoparticles which are completely embedded in the first polymer.

In another aspect, there is provided a method of forming the flexible electronic device in various embodiments of the first aspect, comprising:
forming a first component comprising a first biphasic portion;
forming a second component;
pressing the first component and the second component against each other to form an electrically conductive stretchable interface configured between and in contact with both the first component and the second component,
wherein the electrically conductive stretchable interface comprises the first biphasic portion which is adhered to a portion of the second component,
wherein the first biphasic portion comprises a first polymer having
   (i) a surface partially covered with metal nanoparticles which are partially exposed at the surface, and
   (ii) metal nanoparticles which are completely embedded in the first polymer.

### BRIEF DESCRIPTION OF THE DRAWINGS

The drawings are not necessarily to scale, emphasis instead generally being placed upon illustrating the principles of the present disclosure. In the following description, various embodiments of the present disclosure are described with reference to the following drawings, in which:

FIG. 1A is a schematic diagram illustrating a traditional stretchable hybrid system, where three types of elementary components (also termed “modules” in the present disclosure) are integrated together through interfaces in between.

FIG. 1B shows illustrates a biphasic nano-dispersed (BIND) interface of the present disclosure, wherein gold nanoparticles are immersed in a SEBS polymer matrix. SEBS denotes for styrene ethylene butylene styrene polymer, i.e. a copolymer. The right image is a magnified illustration showing exposed SEBS and gold (Au) on the surface, as well as completely immersed Au, both can aid in providing continuous mechanical and electrical pathways in the layer of BIND interface.

FIG. 1C shows the BIND connection between soft-soft, soft-rigid and soft-encapsulation components. Specifically, the soft-soft modules involve two connecting BIND interfaces. The soft-rigid modules involve one BIND interface connected to another BIND interface fabricated on a rigid substrate. In the soft-encapsulation modules, the encapsulation material pressed onto a BIND interface can be patterned to reveal parts of the BIND interface.

FIG. 1D is a graphical plot illustrating the soft-soft BIND connection exhibits superior electrical (180%) and mechanical (600%) stretchability, compared to traditional conductive pastes used as the connected conventional interface. Error bars are standard deviation (s.d.) from 4 to 5 samples. Compared to traditional commercial pastes, soft-soft BIND connection (e.g. overlapping area: 5 mm width×10 mm length) displayed greater electrical (180%) and mechanical (600%) stretchability.

FIG. 1E shows that the soft-rigid BIND connection (BIND interface-PI) has an electrical stretchability of 200%, larger than traditional connection (PDMS-ACF-PI having only ˜67% stretchability).

FIG. 1F illustrates that the soft-encapsulation BIND connection which involves pressing a SEBS encapsulation layer (˜100 μm thick) onto a BIND interface, displaying a larger peeling force plateau (0.12 N/mm) than a traditional connection formed by pressing PDMS encapsulation material onto a PDMS/Au interface (0.002 N/mm).

FIG. 1G is a schematic of the configuration for traditional connection between soft components. In the traditional connection, the interfaces connected via commercial paste such as ACF (anisotropic conductive film), which suffers from weak bonding and stress concentration upon stretching.

FIG. 1H shows the performance of the traditional connection at ε=0%.

FIG. 1I shows the performance of the traditional connection at ε=50%, wherein electrical failure occurred. Specifically, the gold on interface was peeled off at 50% strain, losing its electrical stretchability.

FIG. 1J shows the performance of the traditional connection at ε=76%, wherein mechanical failure occurred. Specifically, the whole connection was mechanically broken at 76% strain.

FIG. 1K is a schematic of the configuration for the present BIND connection between soft components. The BIND connection without additional pastes shows strong adhesion and high stretchability.

FIG. 1L shows the performance of the BIND connection at ε=0%.

FIG. 1M shows the performance of the BIND connection at ε=180%, which maintains its conductivity until 180% strain.

FIG. 1N shows the performance of the BIND connection at ε=600%, which maintains its mechanical integrity even at more than 600% strain.

FIG. 1O is a plot of the electromechanical performance of the BIND connection at different strains. In both large strain (180% —see right plot) and small strain (<50% —see left plot) region, the BIND connection exhibits high electrical stretchability and low resistance change (remains conductive), compared to traditional connection using commercial pastes. The enlarged figure (on right) shows that for small strain (<50%), BIND connection exhibits much smaller resistance change (<4 times change at 50% strain), and for large strain the BIND connection still maintains a significantly high electrical stretchability even under 180% strain in contrast to a traditional soft-soft connection.

FIG. 1P shows the area of interest in Auger spectroscopy analysis, showing no damage prior to the measurement. The surface morphology of non-conductive interface (left image), BIND interface (center image) and non-adhesive interface (right image), are consistent with their morphology in FIG. 10A to 10C.

FIG. 1Q shows the analysis process to deconvolute Cu KLL and Au MNN signal, and eliminate the adventitious carbon. The original and differentiated Auger survey spectra exhibit C element from polymer, and Au element from Au nanoparticles (left image). In the intensity profile of Au on Si wafer sample, the original C KLL signal is noted as C (total), while the corrected C KLL signal after deconvolution is noted as C (real component) (center image). In this Au on Si sample, the atomic concentration as function of depth shows the deconvolution effectively exclude the effect from Au NVV signal (right image). Besides, it also shows the C concentration before etching (0^{th}, cycle) mainly comes from adventitious carbon, so the 0^{th} cycle was excluded in later analysis.

FIG. 1R shows the differentiated scan spectrum as function of depth, suggesting different inner structure. The non-conductive interface (top left) and BIND interface (top right) exhibited simultaneous C KLL signal and Au MNN signal in the first few cycles, suggesting interpenetrating polymer and metal phases. The non-adhesive interface without C KLL signals in the first few cycles indicates stacking Au nanoparticles without polymer immersion (bottom left). The Au on Si wafer showed no C KLL signals until 40 cycle, excluding the effect of adventitious carbon (bottom right).

FIG. 2A shows the direction for adhesion AFM mappings of a top-viewed BIND interface which are further illustrated through FIG. 2B to 2D. The colour scale below FIG. 2A is meant for FIG. 2B to 2D.

FIG. 2B is an adhesion AFM mapping that shows decreasing ratio of polymer/metal phase from a non-conductive interface, which explains the macroscopic electro-mechanical properties. The non-conductive interface has a higher polymer to metal ratio on its surface compared to FIGS. 2C and 2D.

FIG. 2C is an adhesion AFM mapping that shows decreasing ratio of polymer/metal phase from a BIND interface, which explains the macroscopic electro-mechanical properties. The surface of the BIND interface has both exposed polymer (lighter shaded regions) and metal (darker shaded regions) phases.

FIG. 2D is an adhesion AFM mapping that shows decreasing ratio of polymer/metal phase from a non-adhesive interface, which explains the macroscopic electro-mechanical properties. The non-adhesive interface has a higher metal to polymer ratio compared to FIGS. 2B and 2C.

FIG. 2E shows modulus AFM mapping of cross-sectional viewed BIND interface, demonstrating the biphasic nanostructure inside. FIG. 2E is a magnified view of a portion of the BIND interface of FIG. 2F, showing decreasing ratio of polymer to metal (i.e. increasing denser metal phase in a non-conductive connection). Scale bar denotes 400 nm.

FIG. 2F is a modulus AFM mapping that exhibits increasingly denser metal phase from a non-conductive connection. Scale bar denotes 400 nm.

FIG. 2G is a modulus AFM mapping that exhibits increasingly denser metal phase from the present BIND connection. Scale bar denotes 400 nm.

FIG. 2H is a modulus AFM mapping that exhibits increasingly denser metal phase from a non-adhesive connection. Scale bar denotes 400 nm.

FIG. 2I is a schematic showing increasing density and decreasing depth of Au nanoparticles in non-conductive interface, BIND interface, and non-adhesive interface. Specifically, FIG. 2I shows a schematic of the surficial, inter-penetrating and bottom layers of the interface.

FIG. 2J plots the corresponding atomic concentration measurements of FIG. 2I from Auger spectroscopy, showing increasing density and decreasing depth of Au nanoparticles in non-conductive interface, BIND interface, and non-adhesive interface. The Au nanoparticles are evenly distributed within the polymer phase near the surface of the BIND interface. Au nanoparticles in the non-conductive interface are sparsely distributed while those in the non-adhesive interface are densely packed at the surface.

FIG. 2K is a violin plot showing via Auger depth profiling that in the present BIND interface, the interpenetrating layer has atomic concentration of 75% C and 25% Au.

FIG. 2L is a box plot that shows the penetrating depth of Au for ˜70 etching cycle for the present BIND interface, which is between the non-conductive interface and non-adhesive interface. Data is obtained from 3 to 7 samples (middle line/hollow point: median; box limits: upper and lower quartiles; whiskers: 1.5× interquartile range).

FIG. 2M is a plot of surficial and cross-sectional sheet resistance, consistent with polymer/metal ratios for all the interfaces. Error bars are standard deviation from 3 samples.

FIG. 2N is a box plot that shows the robustness of BIND interface against tape peeling, owning to its interpenetrating nanostructure. Unlike traditional PDMS interface, the present BIND interface is able to withstand scotch tape peeling as shown by its lower change in resistance. Data is obtained from 3 to 7 samples (middle line/hollow point: median; box limits: upper and lower quartiles; whiskers: 1.5×interquartile range).

FIG. 2O shows the electrical performance of three interfaces as a function of nanoparticle evaporation rate and thickness. Specifically, FIG. 2O is an electrical stretchability curve showing that non-conductive interface was fabricated under lowest evaporation rate (0.1-0.2 Å/s) and thickness (45 nm). As evaporation rate and thickness increase, the electrical stretchability on single interface first become highest (180%), then decreased to ˜40%. Error bars are standard deviation from 3 to 5 samples.

FIG. 2P shows the electrical performance of three interfaces as a function of nanoparticle evaporation rate and thickness. Specifically, FIG. 2P is a contour colour plot showing that non-conductive interface was fabricated under lowest evaporation rate (0.1-0.2 Å/s) and thickness (45 nm). As evaporation rate and thickness increase, the electrical stretchability on single interface first become highest (180%), then decreased to ˜40%.

FIG. 2Q shows the electrical stretchability curves exhibiting similar tendency for single interface (as a function of nanoparticle evaporation rate and thickness). Only limited combination of evaporation rate (0.1-1.0 Å/s) and thickness (45-60 nm) forms the BIND connection, while lower rate and thickness results in non-conductive connection, and higher ones in non-adhesive connection. Error bars are standard deviation from 3 to 5 samples.

FIG. 2R shows the contour colour map exhibiting similar tendency for single interface (as a function of nanoparticle evaporation rate and thickness). Only limited combination of evaporation rate (0.1-1.0 Å/s) and thickness (45-60 nm) forms the BIND connection, while lower rate and thickness results in non-conductive connection, and higher ones in non-adhesive connection.

FIG. 2S shows the mechanical performance of three connections. The stretchability curves demonstrate monotonically decreasing mechanical stretchability, with increasing evaporation rate and thickness. This is because the mechanical adhesion originates from the exposed polymer, so larger rate and thickness cover more area on the interface with Au nanoparticles, and lower the bonding ability of interfaces. Error bars are standard deviation from 3 to 5 samples.

FIG. 2T shows the mechanical performance of three connections. The contour color map demonstrates monotonically decreasing mechanical stretchability, with increasing evaporation rate and thickness. This is because the mechanical adhesion originates from the exposed polymer, so larger rate and thickness cover more area on the interface with Au nanoparticles, and lower the bonding ability of interfaces.

FIG. 3A is a schematic of a BIND electrode of the present disclosure, comprised of the ultrathin, conformal electrode with encapsulation, and connected to the thick, robust wiring with encapsulation via the present BIND interface. The SEBS encapsulation protects the electrode, exposing only the contact areas.

FIG. 3B shows an application process of BIND electrode for in vivo neuromodulation. BIND electrode was wrapped around the tissue where ultrathin component in direct contact with surface, and was folded over before adhered to itself.

FIG. 3C is a BIND electrode of the present disclosure which exhibits both conformal contact on tissue and robust wiring, while ultrathin electrode crumpled easily, and thick electrode could not wrap conformably to tissue.

FIG. 3D shows in vivo CMAP signals of successful recording via all three electrodes from the peroneus longus muscle in a rat leg, wherein the peroneal nerve was stimulated using a 2-channel polyimide electrode.

FIG. 3E shows stimulation artifact and evoked CMAP from BIND electrode, demonstrating high repeatability, i.e. consistently displaying a pattern of stimulation artefact and evoked CMAP. Error bars are standard deviation from 10 peaks.

FIG. 3F shows from the CMAP recordings that the BIND electrode exhibits lowest baseline noise and highest signal-to-noise ratio (SNR), indicating the significance of modular configuration. Error bars are standard deviation from 10 peaks.

FIG. 3G shows the universality of BIND electrodes in an in vivo neuromodulation using simultaneous CMAP stimulation and recording. Specifically, the CMAP recordings fromperoneus longus muscle was obtained via a 2-channel (CH1 and CH2) BIND electrode after the common peroneal nerve was stimulated using a second BIND electrode.

FIG. 3H shows the universality of BIND electrodes in an in vivo neuromodulation using cortex ECoG recording. Observably, ECoG recording from epilepsy rat cortex has higher power in the ECoG frequency range than those from healthy rats.

FIG. 3I shows the universality of BIND electrodes in an in vivo neuromodulation using bladder stimulation. Bladder pressure changes when BIND electrode sutured on the bladder wall stimulates the bladder muscle to contract and release urine.

FIG. 3J shows that the adhesion strength of BIND connection was not much influenced by pressing time, demonstrating its usage convenience. The BIND connection can be conveniently formed by short pressing time of 1 s. Simply pressing for 1 s at a low pressure of 0.001 MPa (i.e. a normal finger press) is enough to connect BIND interfaces together. Error bars are standard deviation from 3 to 4 samples.

FIG. 3K shows that the adhesion strength of BIND connection was not much influenced by pressure, demonstrating its usage convenience. The BIND connection can be conveniently formed by low pressure of 0.001 MPa, which means simply finger pressing can construct the BIND interfaces together for a connection. Consistent with the results in FIG. 2O to 2T, non-adhesive connections (120 nm thick Au layer) cannot adhere regardless of pressure. Error bars are standard deviation from 3 to 4 samples.

FIG. 3L shows that the non-adhesive connection (Au 120 nm thickness) cannot adhere to each other at all, no matter how long the pressing time was. Moreover, the peeling direction, lap shear and 90° C. peeling in specific, has little influence on adhesion strength, showing large usage freedom in application. In all other experiment, only lap shear test was employed as it is more common for practical applications. Error bars are standard deviation from 3 to 4 samples.

FIG. 3M shows the anti-tearing performance of BIND connection, showing its resistance against cut. The BIND connection and ACF connection were initially cut (˜1 mm) in the middle. The lap shear test shows that the BIND connection still maintains high tearing force (>1.4 N) while the ACF connection easily broken in presence of initial cut. The error bars are s.d. from 3 samples.

FIG. 3N shows the anti-tearing performance of BIND connection, showing its resistance against cut. The BIND connection and ACF connection were initially cut (˜1 mm) in the middle. The lap shear test shows that the BIND connection still maintains high mechanical stretchability (>500%) while the ACF connection easily broken in presence of initial cut. The error bars are s.d. from 3 samples.

FIG. 3O shows the anti-tearing performance of BIND connection, showing its resistance against cut. Specifically, FIG. 3O shows a photo of ACF connection with initial cut broken easily at the cut location at 50% strain.

FIG. 3P shows the anti-tearing performance of BIND connection, showing its resistance against cut. Specifically, FIG. 3P shows the initial cut in BIND connection can open and endure stretchability of 500%.

FIG. 3Q shows the tape peeling test for the robustness of BIND interface against mechanical abrasion. A scotch tape was used to see whether the interface can be damaged by the tape.

FIG. 3R shows the BIND interface shows high resistance against tape peeling, while the surface remains nearly unbroken after tape peeling. This is because the interpenetrating polymer and metal phase largely enhances its robustness.

FIG. 3S shows the gold easily peeled off by tape. In comparison with FIG. 3R, the conventional PDMS/Au interface has weak bonding between gold nanoparticles and substrate, thus the gold were easily peeled off by tape, leaving the transparent substrate.

FIG. 3T plots for the present BIND connection to show high cyclic durability over 500 cycles, under 20% and 50% tensile strain, bending radius of 1 mm and 2 mm, and twisting angle of 600 and 90°.

FIG. 3U shows that the mechanical stretchability of the present BIND connection is basically unchanged after being subject to the different contortions. Error bars are standard deviation from 3 to 4 samples.

FIG. 4A is a schematic of an electromyography (EMG) electrode array that includes the BIND electrode of the present disclosure, which is composed of: the ultrathin, conformal electrode with encapsulation, and the thick, robust wiring with encapsulation, and the customized printed circuit board (PCB).

FIG. 4B shows a photo of an electromyography (EMG) electrode array that includes the BIND electrode on human arm, with enlarged view of BIND connection and encapsulation. Inset: 21-channel electrode pattern. Enlarged photos: three types of BIND connections in this system, including (i) flex-soft, (ii) soft-soft and (iii) soft-encapsulation.

FIG. 4C shows a photo of before (top image) and after (bottom image) connection pressing, using an insulating tweezer clamp.

FIG. 4D shows averaged EMG signals from 21 channels obtained from ACF-connected control electrode and the present BIND electrode, when the connection between the wiring and PCB undergoes connection pressing (wherein yellow area (shaded region) represents the pressing action) and release. The circled number indicates different pressing stages.

FIG. 4E shows that the BIND electrode shows much lower pressing artifact of 0.03 mV and releasing artifact of 0.12 mV, superior to ACF-connected electrode. Error bars are standard deviation from 21 channels. Circled numbers correspond to pressing stages in FIG. 4D.

FIG. 4F shows the SNR of action in BIND electrode is largely maintained during pressing (17.2 dB) and recovered after pressing (20.3 dB), where the control suffers from signal loss (−0.06 dB) during pressing, and poor recovery (7.56 dB) afterwards. Error bars are standard deviation from 21 channels. Circled numbers correspond to pressing stages in FIG. 4D.

FIG. 4G shows the intensity contour mapping of 21-channel EMG signals for clench gesture in air.

FIG. 4H shows the intensity contour mapping of 21-channel EMG signals for index finger stretching underwater.

FIG. 4I illustrates the soft-soft BIND connection, showing the patterning capability and diverse metal material choice. The BIND connection can be employed to connect patterned modules, because no additional paste is required (see left image). The center image shows a photo of patterned BIND connection with 2 channels shows its patterning capability (the inset shows the BIND connection is electrically conductive to light a light emitting diode (LED)). The right image shows the BIND connection can be further expanded to other conductive materials, including silver/silver connection, and silver/gold connection, all of which are able to exhibit robust electrical and mechanical stretchability. Error bars are standard deviation from 3 to 4 samples.

FIG. 4J shows the soft-rigid BIND connection having high electrical (200%) and mechanical (800%) stretchability, and diverse rigid/flex material choice. Using soft-rigid BIND connection, BIND interface and PI cable can be connected with 6 channels, showing its pattern ability (left image). Here the PI cable can be connected to other rigid/flex component by traditional methods in industry such as soldering. This soft-rigid BIND connection needs no additional pastes, compared to conventional connection (center image). Other rigid/flex materials (such as PET, glass and metal) can also be reliably connected to BIND interface with high electrical stretchability (200%) and mechanical stretchability (800%), demonstrating the material choice freedom and versatility (right image).

FIG. 4K illustrates for SEBS encapsulation layer on a BIND interface (see top image), it shows stronger interface toughness (0.24 N/mm) compared to various encapsulation layer on traditional PDMS/Au interface. All encapsulation layers are ˜100 μm thick. The SEM images of BIND encapsulation on a pair of electrodes, exposing two pads for signal collection are shown in the bottom image. SEBS encapsulation layer is ˜400 nm thick and magnified view on bottom right shows the precise patterned opening.

FIG. 4L shows a fabrication process of ultrathin, conformal electrode with encapsulation. The ultrathin encapsulation layer was gently placed on top of conformal electrode, leaving exposed pads to contact with skin, and one end for BIND connection.

FIG. 4M shows a fabrication process of thick, robust wiring with encapsulation, where one end was exposed for BIND connection, and the other end was connected to stimulator/recording equipment.

FIG. 4N shows in vivo neuromodulation electrode was integrated via BIND connection, by pressing the exposed BIND interfaces together face-to-face.

FIG. 4O shows that in terms of resolution, electrical stretchability of both single BIND interface (left plot) and BIND connection (center plot) decreases with reduced line width (overall width 5 mm), which is because the microcracks have larger influence on electron pathway for narrow interface. Mechanical stretchability of BIND connection (right plot) was almost unchanged for different line width, while the gap width was kept the same as line width, and overall width kept as 5 mm. Resolution of BIND encapsulation can reach 0.1 mm, where the encapsulation layer with through hole was fabricated using SU-8 pillars as mold (right plot). Error bars standard deviation from 3 to 4 samples.

FIG. 5A shows the enlarged AFM mapping on BIND interface, showing Au nanoparticles protrudes over the polymer level. The height mapping of BIND interface shows height variation of ˜14 nm. Scale bar denotes 40 nm.

FIG. 5B shows the normalized line profile, which clearly shows that the adhesion and height are out of phase with each other. Because the high/low adhesion region corresponds to polymer/metal phase, respectively, the line profile suggests that the metal phase is always higher than polymer phase. This height relation matches well with the model, which shows half-immersed gold nanoparticles are higher than polymer level.

FIG. 5C shows the top-viewed AFM images of three interfaces, showing decreasing polymer/metal ratio on the surface for the non-conductive interface, BIND interface and non-adhesive interface, which is consistent with the adhesion mapping for FIG. 2E to 2H. Due to the large modulus difference, polymer and metal phase can be easily distinguished and are labeled in blue (darker shade regions) and yellow (lighter shade regions), respectively. The AFM modulus mapping clearly shows that, the metal/polymer ratio increases from non-conductive interface (top left image), to BIND interface (top center), to non-adhesive interface (top right), which is consistent with the adhesion mapping (FIG. 2B to 2D). Because the self-healing polymer introduces adhesion and the metal provides conductivity, the metal/polymer ratio also well explained their electromechanical properties. The height AFM mapping of the non-conductive interface (top center) and BIND interface (top right) shows similar height variation. This is probably because in both cases, the height variation comes from the half-immersed gold nanoparticles. On the other hand, the height variation in non-adhesive interface is higher (bottom left), because it comes from the stacking gold nanoparticles on top of the polymer, instead of half-immersed nanoparticles. Scale bars denote 100 nm.

FIG. 5D is a schematic of simultaneous CMAP stimulation and recording, using a BIND electrode of the present disclosure. In this configuration, both stimulation and recording electrodes are BIND electrode, while the ultrathin electrode provides conformal contact, and the thick wiring provides robustness. The BIND stimulation electrode conveys electrical pulses to peroneal nerve so as to evoke peroneus longus muscle contraction. The generated subcutaneous CMAP signal was then collected by 2-channel BIND recording electrode wrapping on peroneus longus muscle.

FIG. 6A illustrates for enlarged AFM mapping on BIND connection, showing sandwiched metal phase inside polymer phase. The height mapping shows the existence of polymer phase inside the metal phase, indicating an interpenetrating nanostructure. Scale bar denotes 100 nm.

FIG. 6B illustrates for enlarged AFM mapping on BIND connection, showing sandwiched metal phase inside polymer phase. The height mapping shows the existence of polymer phase inside the metal phase, indicating an interpenetrating nanostructure. Scale bar denotes 100 nm.

FIG. 6C illustrates the cross-sectional AFM images of three connections, showing decreasing penetrating Au nanoparticles inside in non-conductive, BIND and non-adhesive connection. The non-conductive connection was prepared by pressing the corresponding interface together face-to-face. The non-adhesive interface cannot bind to each other, due to the lack of surface-exposed SEBS polymer. Thus, cross-sectional AFM was performed on connection composed of epoxy and non-adhesive interface. Scale bar denotes 400 nm.

FIG. 6D illustrates the cross-sectional AFM images of three connections, showing decreasing penetrating Au nanoparticles inside in non-conductive, BIND and non-adhesive connection. The BIND connection was prepared by pressing the corresponding interface together face-to-face. The non-adhesive interface cannot bind to each other, due to the lack of surface-exposed SEBS polymer. Thus, cross-sectional AFM was performed on connection composed of epoxy and non-adhesive interface. Scale bar denotes 400 nm.

FIG. 6E shows the non-adhesive interface was cut from the connection image, using the white dashed line in height mapping as boundary. Scale bar denotes 400 nm.

FIG. 6F is an adhesion AFM mapping of cross section, showing interpenetrating polymer and metal phases in non-conductive connection. Scale bar denotes 400 nm.

FIG. 6G is an adhesion AFM mapping of cross section, showing interpenetrating polymer and metal phases in the BIND connection. Scale bar denotes 400 nm.

FIG. 6H shows the mapping of non-adhesive interface shows compact metal phases, indicating nearly no penetrating Au nanoparticles. Scale bar denotes 400 nm.

FIG. 6I shows from the conductivity AFM mapping there is lower conductivity in non-conductive connection than BIND connection (compare to FIG. 6J). Scale bar denotes 400 nm.

FIG. 6J shows from the conductivity AFM mapping there is higher conductivity in the BIND connection than the non-conductive connection (compare to FIG. 6I). Scale bar denotes 400 nm.

FIG. 6K shows the non-adhesive interface shows little conductivity, probably because there was only one layer of gold nanoparticles in its cross section. Scale bar denotes 400 nm.

FIG. 6L shows the fabrication process of ultrathin, conformal electrode with encapsulation, where all electrode pads are exposed for skin contact, and one end is exposed for BIND connection.

FIG. 6M shows the Fabrication process of thick, robust wiring with encapsulation, where two ends are exposed for BIND connection via removal of sandwiched paper.

FIG. 6N shows the fabrication of customized PCB with BIND interface, where one end is compatible to biopotential signal collector via cable socket, and the other end was BIND interface for connection.

FIG. 6O shows the three parts are assembled together by simply pressing the BIND interface together, to obtain 21-channel EMG electrode system.

FIG. 7A shows the BIND electrode wrapping on common peroneal nerve (i), sciatic nerve (ii) and peroneus longus muscle (iii), respectively, shows that the suture-free procedure is compatible with different organs to provide conformal contact. The BIND electrode can also be placed on cerebral cortex, showing the conformal contact without wrapping (iv). It can also be sutured on bladder wall, where the conformal contact allows the electrode to shrink together with bladder (v). This bladder experiment can be further improved in the future, to wrap the BIND electrode around the bladder to avoid suture.

FIG. 7B shows the BIND electrode for sciatic nerve stimulation, showing strong resistance against mechanical interference, including touching and pulling. The BIND electrode was wrapped on sciatic nerve, with the ultrathin, conformal electrode in direct contact with nerve, and the thick, robust wiring relaying the signal via BIND connection. The stimulated effectiveness was measured by analyzing the moving distance of rat ankles. Mechanical interference was applied, including touching sciatic nerve with tweezers (i), pulling cathode (ii) and anode (iii) in turn. The statistics analysis shows little difference in rat ankle movement under mechanical interference, compared to no interference situation (iv).

FIG. 7C shows the impedance of BIND interface, demonstrating low impedance and high stability under strain. The 3D mapping of BIND interface impedance, showing typical impedance value of metal-based stretchable electrode (i). In frequency range of interest (e.g. ECoG 10-50 Hz, EMG 10-500 Hz), the impedance changed little until 70% strain (ii). This impedance test demonstrates the interfacial impedance in between organ/skin and ultrathin electrode, which can be improved via various methods (e.g. coating other materials, increasing surface area) in future study. The BIND connections construct electrical pathway by Ohmic contact between Au nanoparticles, so its influence on impedance is the same as its electrical resistances in DC tests.

FIG. 8A shows the experimental setup and performance of simultaneous CMAP stimulation and recording. The photo of BIND stimulation and recording setup, with BIND electrodes wrapping on common peroneal nerve and peroneus longus muscle, respectively.

FIG. 8B shows the stimulation current was applied to common peroneal nerve with a parallel resistance of 250 ohms.

FIG. 8C shows that from the P-P voltage of CMAP under different stimulation current, the evoked CMAP was triggered when stimulation current reached a threshold, then increased and reached a saturation stage.

FIG. 8D shows the spectroscopy of recorded CMAP shows significant power density in typical CMAP frequency range (10-500 Hz).

FIG. 8E shows that the ankle movement was measured by the distance of rat ankle before stimulation.

FIG. 8F shows that the ankle movement was measured by the distance of rat ankle during stimulation.

FIG. 8G shows the BIND electrode on cerebral cortex can successfully detect ECoG in healthy and epilepsy rat. In the 4-channel BIND electrode, the ultrathin part conformally contacts with cerebral cortex, and the thick wiring provides robustness.

FIG. 8H shows the 4-channel ECoG signal of heathy and epilepsy rat exhibits significant difference in both amplitude and frequency.

FIG. 8I shows the BIND electrode for bladder stimulation and recording. For bladder stimulation, the BIND electrode was sutured on bladder wall, and a catheter was inserted into bladder for urination and pressure sensing.

FIG. 8J shows that when the bladder filled with normal saline was stimulated by BIND electrode, the bladder muscle contracted, squeezing the saline solution out, which was detected by the external pressure sensor.

FIG. 8K shows that for bladder recording, BIND electrode was wrapping around the bladder. Normal saline was injected into the bladder through catheter, and the corresponding expansion can be detected by BIND electrode.

FIG. 9A demonstrates for performance under connection pressing. For ACF-connected electrode, the application and release of pressure on connection area 779 induced large noise in EMG signal. The EMG of muscle action under connection pressing can still be detected, yet with large noise, especially in between two actions. Displayed signals in the electrode underwent a 50 Hz notch filter, and a 30 Hz high pass filter.

FIG. 9B demonstrates for performance under connection pressing. For BIND electrode, EMG of muscle action can be clearly detected, in periods of before pressing, during pressing and after pressing. The noise from pressure application has little effect, while the noise from pressure release is a little larger. Displayed signals in the electrode underwent a 50 Hz notch filter, and a 30 Hz high pass filter. In all, the BIND electrode showed much better resistance to mechanical pressure on connection area, compared to ACF-connected electrode of FIG. 9A.

FIG. 9C shows performance under 50% connection stretching. Specifically, FIG. 9C are photographs showing both the BIND electrode and ACF-electrode stretched to 50% strain. One side of connection was fixed via scotch tape, and the other side was manually stretched to the 50% mark. Displayed signals in both electrodes underwent a 50 Hz notch filter, and a 30 Hz high pass filter.

FIG. 9D is a plot of the corresponding quantitative analysis of FIGS. 9C, 9F and 9G. Before strain, both ACF-connected and BIND electrode can detect EMG signal from muscle action (fist clenching). When 50% strain is applied, both electrodes showed interference noise. During strain, the EMG signal from BIND electrode has larger signal-to-noise ratio than that of ACF-connected electrode, indicating the detection capability even under large strain. The release of 50% strain induced larger noise in ACF-connected electrode, and both electrodes can recover the detection capability after strain releasing. The circled numbers correspond to the stages in FIG. 9B.

FIG. 9E is a plot of the corresponding quantitative analysis of FIGS. 9C, 9F and 9G. Before strain, both ACF-connected and BIND electrode can detect EMG signal from muscle action (fist clenching). When 50% strain is applied, both electrodes showed interference noise. During strain, the EMG signal from BIND electrode has larger signal-to-noise ratio than that of ACF-connected electrode, indicating the detection capability even under large strain. The release of 50% strain induced larger noise in ACF-connected electrode, and both electrodes can recover the detection capability after strain releasing. The circled numbers correspond to the stages in FIG. 9B.

FIG. 9F shows for the 21-channel EMG signal using the ACF-electrode under 50% connection stretching.

FIG. 9G shows for the 21-channel EMG signal using the BIND electrode under 50% connection stretching.

FIG. 10A shows the SEM morphology for an interface (non-conductive) demonstrating for microcracks under strain of 0%. Scale bar denotes for 10 μm.

FIG. 10B shows the SEM morphology of a BIND interface demonstrating for microcracks under strain of 0%. Scale bar denotes for 10 μm.

FIG. 10C shows the SEM morphology of a non-adhesive interface demonstrating for microcracks under strain of 0%. Obvious microcracks can be seen. Scale bar denotes for 10 μm.

FIG. 10D shows the SEM morphology for an interface (non-conductive) demonstrating for microcracks under strain of 50%. Scale bar denotes for 10 μm.

FIG. 10E shows the SEM morphology of a BIND interface demonstrating for microcracks under strain of 50%. Scale bar denotes for 10 μm.

FIG. 10F shows the SEM morphology of a non-adhesive interface demonstrating for microcracks under strain of 50%. Scale bar denotes for 10 μm.

FIG. 10G shows the SEM morphology for an interface (non-conductive) demonstrating for microcracks under strain of 100%. The 100% strain images showed larger microcrack opening compared to that of 50% strain, while the non-adhesive interface still shows the largest microcracks. Scale bar denotes for 10 μm.

FIG. 10H shows the SEM morphology of a BIND interface demonstrating for microcracks under strain of 100%. The 100% strain images showed larger microcrack opening compared to that of 50% strain, while the non-adhesive interface still shows the largest microcracks. Scale bar denotes for 10 μm.

FIG. 10I shows the SEM morphology of a non-adhesive interface demonstrating for microcracks under strain of 100%. Obvious microcracks can be seen. The 100% strain images showed larger microcrack opening compared to that of 50% strain, while the non-adhesive interface still shows the largest microcracks. Scale bar denotes for 10 μm.

FIG. 10J shows the adhesion AFM mapping of BIND interface under 50% strain. The adhesion mapping shows that the microcrack tears the whole interpenetrating layer, exposing pure polymer phase within the microcrack opening. Otherwise, if the interpenetrating layer is not completely torn in depth, the metal phase should be seen inside the opening.

FIG. 10K shows the height AFM mapping wherein the microcrack depth is ˜90 nm. It is taken that the depth of interpenetration layer is the same as the microcrack depth, because this depth value is consistent with that of cross-sectional AFM images. This depth value (90 nm) is larger than the expected thermal deposition thickness (45 nm on rigid substrate), attributed by the presence of interpenetration structure.

FIG. 10L shows the corresponding line profile wherein the microcrack depth is ˜90 nm. It is taken that the depth of interpenetration layer is the same as the microcrack depth, because this depth value is consistent with that of cross-sectional AFM images. This depth value (90 nm) is larger than the expected thermal deposition thickness (45 nm on rigid substrate), attributed by the presence of interpenetration structure.

FIG. 10M shows 21-channel EMG signal under in air environment from different gestures. The hand gesture (clench, open, raise and bend) mainly focuses on the overall hand movement, while the finger gesture corresponds to the stretching of each finger. The 50% and 100% maximum voluntary contraction (MVC) was measured by grip dynamometer.

FIG. 10N shows 21-channel EMG signal in underwater environment, from different gestures. Here the BIND electrode was totally immersed into water, including the ultrathin, conformal electrode with encapsulation, the thick, robust wiring with encapsulation, and BIND connection part in customized PCB. The displayed signal underwent a 50 Hz notch filter, and a 30 Hz high pass filter, except that signal under connection pressing/stretching underwent a 50 Hz notch filter, and a 10 Hz high pass filter. The hand and finger gesture can be clearly detected with high signal-to-noise ratio (top image). In underwater environment, the BIND electrode still demonstrates good mechanical resistance, such as connection pressure (bottom left image) and 50% connection strain (bottom right). In the stage of before, during and after mechanical resistance, the BIND electrode can clearly detect EMG signals with high quality. The application and release of connection pressure raised negligible interference noise. The noise raised from application and release of 50% connection strain is a little larger, yet is still small and distinguishable compared to the EMG signal, which may be analyzed and attenuated using advanced algorithm in further study.

FIG. 11A shows the thermal evaporation process which forms the interface that have gold nanoparticles network and SEBS stretchable substrate integrated.

FIG. 11B shows two single electrodes (modules I and II) overlapped face-to-face, forming stretchable interconnect through simple finger pressing on the overlapped area.

FIG. 12A shows the experimental setup for mechanical and electromechanical characterization. The machine stretches the interconnect in vertical direction, and corresponding mechanical load and electrical conductivity were recorded.

FIG. 12B plots the relative resistance change of interconnect, showing the optimized interconnect maintains conductivity even when the interconnect is under ˜160% strain.

FIG. 12C plots the statistic results of mechanical stretchability and adhesion strength of interconnect, showing the robustness and high adhesion.

FIG. 13A is a plot demonstrating that the binder-free and stretchable interconnect can be formed using conductive materials of silver and/or gold. The electrical and mechanical performance shows little difference, which largely expands the application of interconnects.

FIG. 13B plots the mechanical test results demonstrating that the present interconnect is notch-insensitive while the control sample made of PDMS easily breaks in presence of a notch.

FIG. 14A shows the adhesion mapping on the surface of single electrode, showing co-existence of SEBS polymer and Au (gold) nanoparticles on the surface. This is one reason why the present interconnect has both mechanical and electrical bonding. Scale bar denotes 40 nm.

FIG. 14B shows the adhesion mapping and corresponding illustration of samples fabricated by evaporation rate of 0.1 Å/s. Scale bar denotes 40 nm.

FIG. 14C shows the adhesion mapping and corresponding illustration of samples fabricated by evaporation rate of 0.5 Å/s. Scale bar denotes 40 nm.

FIG. 14D shows the adhesion mapping and corresponding illustration of samples fabricated by evaporation rate of 10.0 Å/s. Scale bar denotes 40 nm.

FIG. 15A shows the traditional connection modeled using finite element analysis (FEA). The terms “traditional” and “conventional” are used exchangeably in the present disclosure.

FIG. 15B shows the present paste-free BIND connection modeled using finite element analysis, wherein the same dimensional parameters in experimental study shown in FIG. 15A is used.

FIG. 15C is a nominal strain colour map under 50% total strain showing for traditional connection having sharper and more concentrated strain distribution than the present BIND connection.

FIG. 15D is a nominal strain colour map under 50% total strain showing for the present BIND connection having less sharp and less concentrated strain distribution than the traditional connection.

FIG. 15E shows that compared to conventional connection, the present BIND connection exhibits wider concentrated strain area and smaller maximum strain with the same applied total strain.

FIG. 15F shows that compared to conventional connection, the present BIND connection exhibits wider concentrated strain area and smaller maximum strain with the same applied total strain.

FIG. 15G is a plot of the concentrated strain curves as functions of total strain. The interface tolerance of single interface (conventional: ε_{conv,interface}=188%; BIND: ε_{BIND,interface} 269%) were used to anchor the maximum total strain of connection (conventional: ε_{conv,connection}=50%; BIND: ε_{BIND,connection}=126%).

FIG. 15H shows that the FEA-assisted analysis deduced a larger total strain of BIND connection compared to conventional connection, which is consistent with the experimental results.

FIG. 16A shows the present BIND interface was fabricated on LED with a surface mount device (SMD) packaging. Left inset: detailed structure of LED with BIND interface. Right inset: the edge of BIND interface. Scale bars denote 100 μm.

FIG. 16B shows that the connection using the present interface coupled to an LED exhibits 50% electrical stretchability.

FIG. 16C shows that the connection using the present interface coupled to an LED exhibits is resistant to tweezers pulling.

FIG. 16D shows that conventional connection for LED via ACF tape and liquid metal exhibit either electrical or mechanical stretchability, yet they cannot achieve both at the same time. In comparison, the present BIND connection demonstrates both electrical stretchability and mechanical adhesion. Error bars are standard deviation from 3 to 4 samples.

FIG. 17A is a plot of the in situ overall temperature of the SEBS copolymer achieves 90° C. during deposition.

FIG. 17B is a plot of the nanoscale dynamic mechanical analysis of AFM on SEBS copolymer exhibiting glass transition in 50 to 70° C.

FIG. 17C is a plot showing the decreasing density of SEBS copolymer with elevated temperature in molecular dynamics (MD) simulations.

FIG. 17D are simulation boxes showing physical picture of Au atom flux impinging SEBS copolymer (see leftmost image). FIG. 17D also shows time evolution of interface exhibits Au penetration and nucleation underneath the polymer surface, in early stage of deposition.

FIG. 18A is a plot showing that, on sweaty skin after exercise, SNR from 21-channel BIND-connected EMG electrode slightly increases compared to original non-sweaty skin. The possible reasons for slightly increased signal quality are accumulated sweat between BIND electrode and skin, as well as hydrated stratum corneum and filled sweat ducts, since sweat is essentially a saline solution. It is worth noting that BIND connection or BIND encapsulation is not influenced by sweat during exercise due to its airtightness. Error bars are standard deviations from 21 channels.

FIG. 18B is a plot of on-skin impedance measurement showing slightly decrease in presence of artificial sweat (bi-electrode configuration, electrode area 10*10 mm²), consistent with the SNR change. Enhancing the gas/sweat permeability in BIND electrode is still highly recommended for long-time wearing comfort, which can also prevent the accumulated sweat to short circuit two neighboring electrodes.

FIG. 19A demonstrates for a customized multifunctional circuit, which can be assembled via plug-and-play BIND connection, which simultaneously detects EMG, pressure, and strain signals. The assembled circuit consists of 5 modules: PI PCB, stretchable pressure sensor, stretchable strain sensor, and stretchable EMG electrodes with encapsulation module. These modules are connected through plug-and-play BIND interface with finger pressing for ˜10 s. Inset: photo of customized circuit on arm.

FIG. 19B shows that polyimide (PI) PCB module contains various standardized electronic components inside module, and the present BIND interfaces are used as modular connection points.

FIG. 19C is a plot demonstrating that the circuit of FIG. 19A when worn on the arm, the customized circuit can detect EMG signals.

FIG. 19D are plots demonstrating that the circuit of FIG. 19A when worn on the arm, the customized circuit can detect EMG signals even under pressure exerted (top plot) and bending (bottom plot).

### DETAILED DESCRIPTION

The following detailed description refers to the accompanying drawings that show, by way of illustration, specific details and embodiments in which the present disclosure may be practised.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure relates to a flexible electronic device. For brevity, the flexible electronic device can be exchangeably termed herein a “device” and “present device”. The device is configured and/or operable as an interconnect, hence can be termed herein an “interconnect”. Where the device is configured and/or operable as an electrode, the device can be termed herein an “electrode”.

The present device includes a component, which may be formed of a polymer incorporated with conductive metal nanoparticles. As the polymer and the metal nanoparticles constitute two different phases, this configuration of materials may be referred herein to as a “biphasic nano-dispersed interface” (abbreviated as BIND interface) and for brevity a “biphasic portion”, wherein “nano-dispersed” refers to the nanoparticles dispersed in another phase, e.g. the polymer. When such BIND interface of the component is contacted with another component, a BIND connection may be established. For example, two of aforesaid components each having the BIND interface may be placed together with the BIND interface of each component in contact, forming the BIND connection. In another example, one of aforesaid component can be placed in contact with another component absent of the BIND interface, and the BIND interface in contact with the another component establishes the BIND connection. It follows that in instances where the present device is configured/operable as an interconnect or electrode, the interconnect and electrode may be termed herein a “BIND interconnect” and “BIND electrode”, respectively. The terms “components” and “modules” may be interchangeably used in the present disclosure.

The present device is advantageous over electronic device having traditional interconnects and electrodes. The traditional interconnects and electrodes tend to be formed from traditional methods using stiff binders, such as anisotropic conductive film (ACF), conductive paste or solder, to combine stretchable components or other parts so as to form the resultant electronic device. However, the stiff binders limit the electronic device from stretching and may easily break when stretched, damaging the electronic device. Also, the stiff binders adhere poorly on the stretchable components and parts, rendering the stiff binders to detach easily from the components and parts when the electronic device undergoes stretching. Other traditional means include using liquid metal and self-healing conductive composites but the former is susceptible to leakage problem and the latter cannot bind components/parts with patterns. Compared to the traditional electronic device having the traditional interconnects and electrodes, the present device has one or more of the following advantages.

The present device is easy to use and fabricate, simply overlap two pieces of components (e.g. electrodes) with the biphasic portion face-to-face and gently press the overlapping area using fingers for a short duration (e.g. 10 seconds), and the two components automatically bond together, without compromising any electrical and mechanically properties.

The present device, as an interconnect for example, having components bound together using the biphasic portion are strongly bonded together. The interconnect can maintain its electrical performance even under ˜160% tensile strain and mechanical integrity is maintained even under ˜800% strain.

The fabrication of the present device, as an interconnect for example, having a component with the biphasic portion is straightforward. The interconnect can be fabricated via thermal evaporation of a conductive metal (e.g. gold) nanoparticles onto a thermoplastic elastomer, e.g. SEBS polymer. As thermal evaporation is used, the parameters for fabrication can be configured to render desirable properties of the biphasic portion for the present device to be used in different applications.

The conductive materials that form the nanoparticles are universal in that widely used materials, such as gold and silver, can be used and not restricted to a sole material. That is to say, a combination of gold and silver, or all silver, or all gold nanoparticles can be used to form the biphasic portion. This imparts versatility to the applications of present device which may require certain materials to be solely used.

The biphasic portion of the present device is notch-insensitive. Traditional materials such as polydimethylsiloxane (PDMS), when it has a notch or cut or damaged, then easily breaks when stretched. The biphasic portion is resistant to breakage even when it has a notch or cut, and yet still able to maintain its stretchability and mechanical/electrical properties, understandably rendering the present device to have the same advantage.

Details of various embodiments of the present device and method of forming the present device, and advantages associated with the various embodiments are now described below. Where the embodiments and advantages have been described in the examples section further hereinbelow, they shall not be reiterated for brevity.

In the present disclosure, there is provided a flexible electronic device comprising a first component comprising a first biphasic portion and a second component. The term “flexible” means that the device can be subjected to an extent of any form of contortion, such as twisting, compression, stretching, without compromising its electrical and mechanical properties.

In various embodiments, the first component and the second component are in contact with an electrically conductive stretchable interface configured between the first component and the second component. Such an electrically conductive stretchable interface is exchangeably termed herein as a “BIND connection”, which is mentioned above. In various embodiments, the electrically conductive stretchable interface comprises the first biphasic portion which is adhered to a portion of the second component.

In various embodiments, the first biphasic portion may comprise a first polymer having (i) a surface partially covered with metal nanoparticles which may be partially exposed at the surface, and (ii) metal nanoparticles which may be completely embedded in the first polymer.

In certain non-limiting embodiments, the electrically conductive stretchable interface may be absent of an adhesive paste.

In certain non-limiting embodiments, the portion of the second component adhered to the first biphasic portion comprises a second biphasic portion. This may be in non-limiting instances wherein the first component and the second component are formed of soft (i.e. less rigid) materials or where one of the components is a soft material and the other is a rigid material. In such non-limiting embodiments, the second biphasic portion may comprise a second polymer having (i) a surface partially covered with metal nanoparticles which may be partially exposed at the surface, and (ii) metal nanoparticles which may be completely embedded in the second polymer, wherein the metal nanoparticles of the first biphasic portion and the second biphasic portion, which are partially exposed, are in contact. In such non-limiting embodiments, the second polymer may comprise styrene ethylene butylene styrene or styrene-butadiene.

In various embodiments, the first polymer may comprise styrene ethylene butylene styrene or styrene-butadiene.

In various embodiments, the metal nanoparticles may comprise gold and/or silver.

In various embodiments, the first polymer and the metal nanoparticles residing (i) proximal to the surface of the first polymer and (ii) at a depth of up to 10 nm (e.g. up to 5 nm, up to 1 nm) from the surface of the first polymer, may have a weight ratio of 40:60 to 60:40 (e.g. 50:50).

In certain non-limiting embodiments, the first polymer and the metal nanoparticles residing (i) proximal to the surface of the first polymer and (ii) at a depth of up to 10 nm (e.g. up to 5 nm, up to 1 nm) from the surface of the first polymer may have a weight ratio of 40:60 to 60:40 (e.g. 50:50), and wherein the first polymer and metal nanoparticles residing at a depth of more than 10 nm and up to 100 nm from the surface of the first polymer have a weight ratio of 30:70 to 70:30 (e.g. 40:70 to 70:40, 50:70 to 70:50, 60:70 to 70:60, 50:50).

In certain non-limiting embodiments, the second polymer and the metal nanoparticles residing (i) proximal to the surface of the second polymer and (ii) at a depth of up to 10 nm (e.g. up to 5 nm, up to 1 nm) from the surface of the second polymer, may have a weight ratio of 40:60 to 60:40 (e.g. 50:50).

In certain non-limiting embodiments, the second polymer and the metal nanoparticles residing (i) proximal to the surface of the second polymer and (ii) at a depth of up to 10 nm (e.g. up to 5 nm, up to 1 nm) from the surface of the second polymer may have a weight ratio of 40:60 to 60:40 (e.g. 50:50), and wherein the second polymer and metal nanoparticles residing at a depth of more than 10 nm and up to 100 nm from the surface of the second polymer may have a weight ratio of 30:70 to 70:30 (e.g. 40:70 to 70:40, 50:70 to 70:50, 60:70 to 70:60, 50:50).

In certain non-limiting embodiments, wherein the metal nanoparticles, which are completely embedded in the first polymer and the second polymer, may be present in the first polymer and the second polymer up to a depth of 80 nm, 90 nm, 100 nm, etc.

In various non-limiting embodiments, the metal nanoparticles may penetrate to a depth of about 100 nm from the surface, as shown in cross-sectional AFM mapping. The nano-dispersed biphasic portion (or known herein as layer) may have a thickness of about 100 nm, wherein beyond 100 nm constitutes solely the polymer phase (i.e. an insulating substrate). In such instances, electricity may only conduct in the 100 nm-thick biphasic portion. For example, electricity may conduct horizontally in the biphasic portion, i.e. across x-y plane, and also conduct vertically to the 100 nm depth, which is sufficient for use as an interconnect and electrode in various applications.

In certain non-limiting embodiments, the first component and the second component may have identical rigidity. In certain non-limiting embodiments, the first component may have a higher rigidity than the second component.

In certain non-limiting embodiments, the second component may be an encapsulation layer.

In certain non-limiting embodiments, the first component having the higher rigidity than the second component may comprise polyimide, polyethylene terephthalate, glass, or silicon.

In various embodiments, the flexible electronic device may be an electrode or an interconnect. The electrode may be a neuro-modulation electrode, or a 21-channel electromyography electrode, attachable to a surface of a skin.

The present disclosure also relates to a method of forming the flexible electronic device described in various embodiments of the first aspect. Embodiments and advantages described for the device of the first aspect can be analogously valid for the present method subsequently described herein, and vice versa. Where the various embodiments and advantages have already been described above and in the examples hereinbelow, they shall not be iterated for brevity.

The method may comprise forming a first component comprising a first biphasic portion, forming a second component, pressing the first component and the second component against each other to form an electrically conductive stretchable interface configured between and in contact with both the first component and the second component, wherein the electrically conductive stretchable interface may comprise the first biphasic portion which is adhered to a portion of the second component, wherein the first biphasic portion may comprise a first polymer having (i) a surface partially covered with metal nanoparticles which may be partially exposed at the surface, and (ii) metal nanoparticles which may be completely embedded in the first polymer.

In various embodiments, forming the first component may comprise arranging the first polymer to face a metal source, and heating the metal source to evaporate metal from the metal source to have metal nanoparticles incorporated to the first polymer, thereby rendering the first biphasic portion.

In certain non-limiting embodiments, forming the second component may comprise arranging a second polymer to face a metal source, and heating the metal source to evaporate metal from the metal source to have metal nanoparticles incorporated to the second polymer, thereby rendering a second biphasic portion in the portion of the second component.

In certain non-limiting embodiments, when the first component has a higher rigidity than the second component, forming the first component may comprise providing a rigid substrate, treating the rigid substrate with oxygen plasma prior to contacting the rigid substrate with an organosilane, forming the first polymer on the rigid substrate, arranging the rigid substrate to have the first polymer face a metal source, and heating the metal source to evaporate metal from the metal source to have metal nanoparticles incorporated to the first polymer, thereby rendering the first biphasic portion.

In certain non-limiting embodiments, when the first component has a higher rigidity than the second component, forming the second component may comprise arranging a second polymer to face a metal source, and heating the metal source to evaporate metal from the metal source to have metal nanoparticles incorporated to the second polymer, thereby rendering a second biphasic portion in the portion of the second component.

In certain non-limiting embodiments, when the second component is an encapsulation layer, forming the first component may comprise arranging the first polymer to face a metal source, and heating the metal source to evaporate metal from the metal source to have metal nanoparticles incorporated to the first polymer, thereby rendering the first biphasic portion.

In certain non-limiting embodiments, when the second component is an encapsulation layer, forming the second component may comprise providing an encapsulation material as the second component.

In certain non-limiting embodiments, when the second component is an encapsulation layer, forming the second component may comprise providing a substrate, treating the substrate with oxygen plasma prior to contacting the substrate with an organosilane, and depositing an encapsulation material on the substrate to form the encapsulation layer.

In certain non-limiting embodiments, where the flexible electronic device is a neuro-modulation electrode or a 21-channel electromyography electrode, forming the first component may comprise providing a rigid substrate, treating the rigid substrate with oxygen plasma prior to contacting the rigid substrate with an organosilane, forming the first polymer on the rigid substrate, arranging the rigid substrate to have the first polymer face a metal source, heating the metal source in the presence of a mask to evaporate metal from the metal source to have metal nanoparticles incorporated to the first polymer according to a pattern defined by the mask, thereby rendering the first biphasic portion, and depositing an encapsulation material on the first polymer in a manner which exposes the first biphasic portion.

In certain non-limiting embodiments, where the flexible electronic device is a neuro-modulation electrode or a 21-channel electromyography electrode, forming the second component may comprise arranging a second polymer to face a metal source, and heating the metal source to evaporate metal from the metal source to form metal lines on the second polymer, and depositing an encapsulation material to cover the metal lines except for two opposing ends of each of the metal lines.

The word “substantially” does not exclude “completely” e.g. a composition which is “substantially free” from Y may be completely free from Y. Where necessary, the word “substantially” may be omitted from the definition of the present disclosure.

In the context of various embodiments, the articles “a”, “an” and “the” as used with regard to a feature or element include a reference to one or more of the features or elements.

In the context of various embodiments, the punctuation “.”, and the terms “about” and “approximately” as applied to a numeric value encompasses the exact value and a reasonable variance, e.g. ±20%, 10%, 5%, 1%, 0.5%, 0.1%, etc.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

### EXAMPLES

The present disclosure relates to a flexible electronic device which addresses the problem of stretchable devices having poor interconnects. The present flexilbe electronic device includes a binder-free stretchable interconnect that is straightforward in use and economical for fabrication. In brief, the flexible electronic device may include overlapping of two single electrodes face-to-face, which can be pressed together using as simple as human fingers for a short duration, e.g. 10 seconds, and the overlapped area becomes bonded together automatically, forming the binder-free stretchable interconnect of the present device. Such a device having aforesaid interconnect is both stretchable and flexible without compromising electrical and mechanical properties. With regard to electrical performance, the present flexible electronic device maintains it electrical conductivity even when the interconnect therein is stretch to ˜160% strain. With regard to mechanical performance, the present flexible electronic device maintains it mechanical integrity even when the interconnect therein is stretch to ˜800% strain.

The present disclosure also relates to a method of forming the present flexible electronic device. The fabrication method is straightforward. The method may involve thermal evaporation of a metal, such as gold, onto a supramolecular polymer, e.g. styrene ethylene butylene styrene (SEBS) polymer. The fabrication parameters and the device performance can be conveniently configured to suit needs of various applications. Moreover, the interconnect is suitable for construction using different conductive materials, including gold and silver. The interconnect is also insensitive to mechanical damage, which is helpful in practical applications. Besides, the underlying mechanism was investigated in order to efficiently modify the interconnect properties.

The present device may be a straightforward and reliable means to connect various stretchable device. The present device has great potential to integrate different stretchable modules in a lego-like way, which largely increases productivity of producers and design freedom of users.

The present device, its uses and method of fabrication are described in further details, by way of non-limiting examples, as set forth below.

### Example 1: General Introduction of a BIND Connection of the Present Disclosure

Comprised of soft, rigid and encapsulation components, stretchable hybrid devices can provide implantable/on-skin high-fidelity monitoring, owing to their mechanical compliance and data processing capability. However, the interfaces between different components tend to suffer from stress concentration, resulting in low stretchability and ultimately debonding failure. The present disclosure describes for a biphasic, nano-dispersed (BIND) interface that includes interpenetrating metal and polymer phases, rendering the integration of stretchable hybrid device with high stretchability and robustness. Such interface can be used in between soft-soft components (mechanical and electrical stretchability of 600% and 200%, respectively), soft-rigid components (various rigid materials including polyimide (PI), polyethylene terephthalate (PET), silicon (Si)) and soft-encapsulation components (peeling force of up to 0.454 N). The present BIND connection can integrate the in vivo neuromodulation device with mechanical conformality and robust wiring, showing advantages of modular design over an one-piece design. A more complex device via BIND connection—the 21-channel on-skin EMG electrode—exhibited high resistance against mechanical interference and waterproofness, achieving high signal fidelity even in underwater environment.

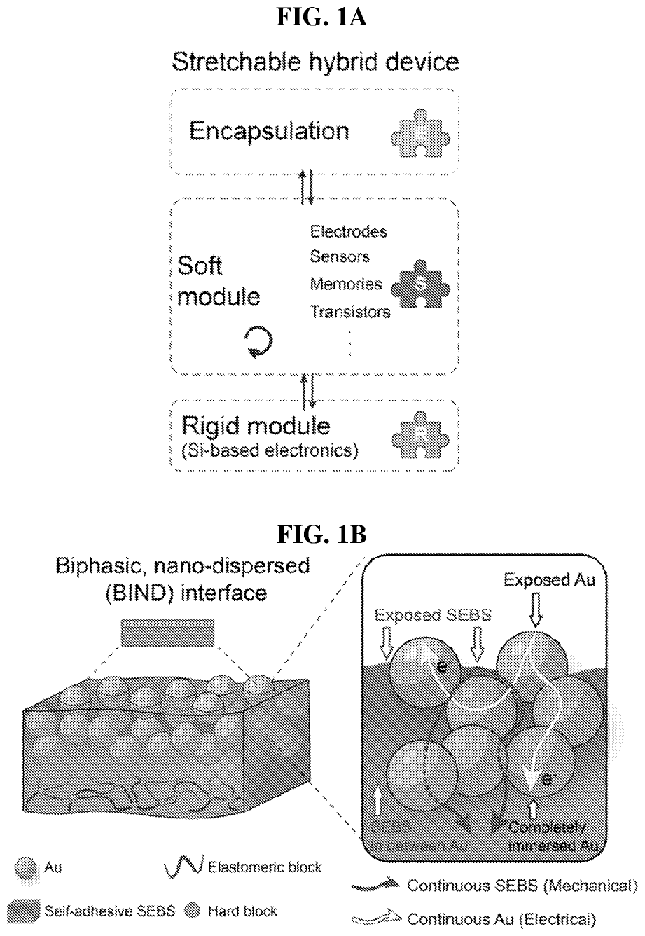

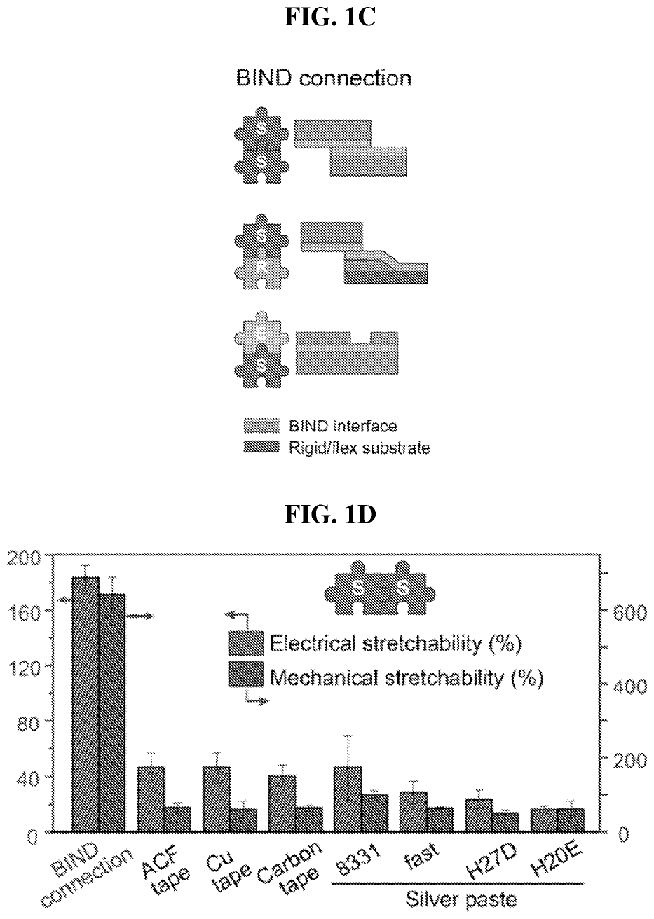

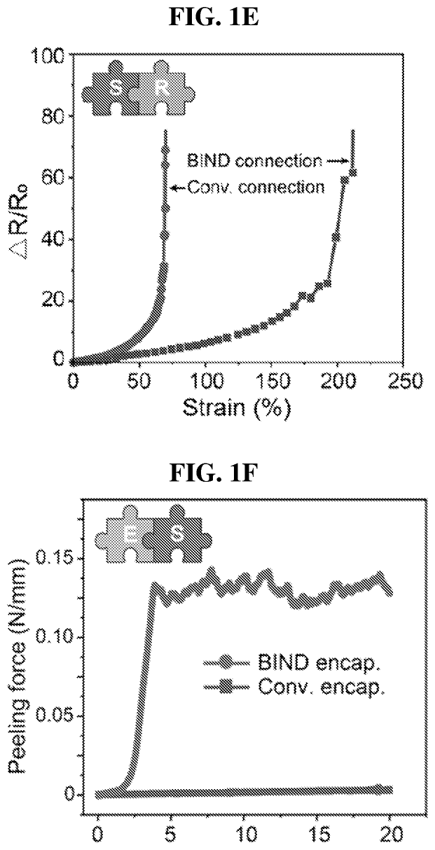

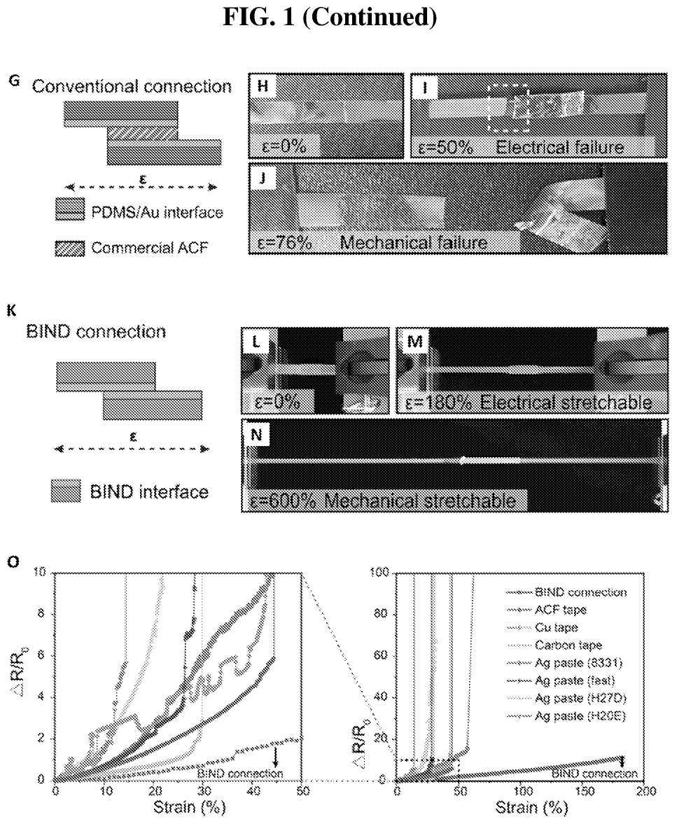

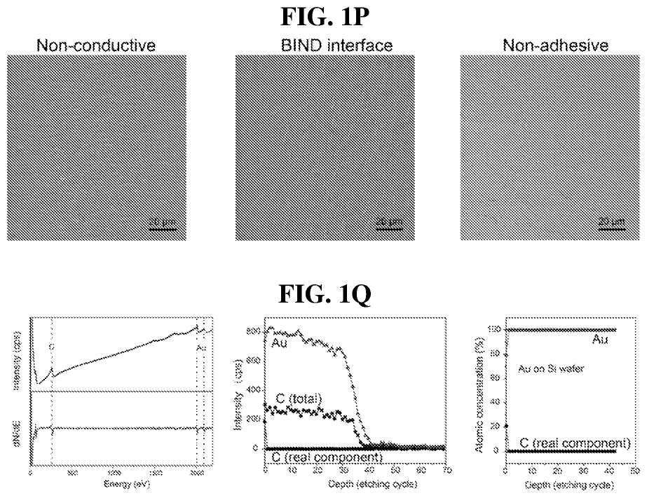

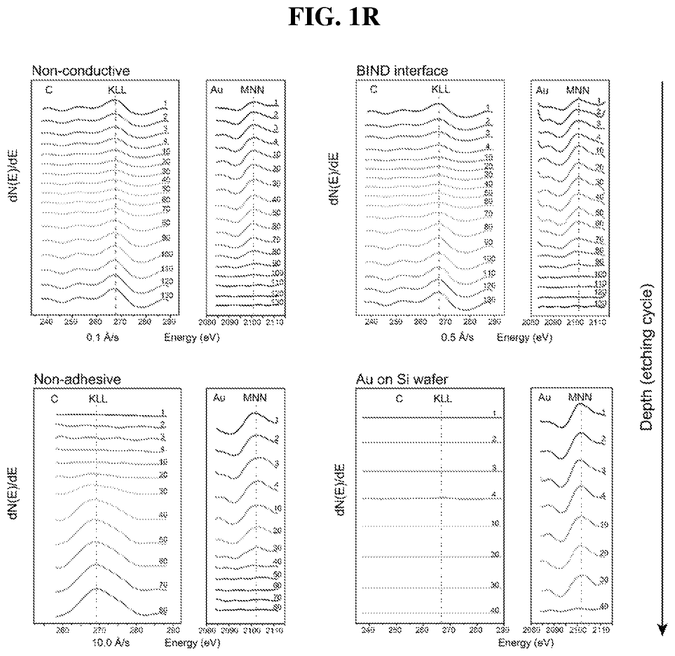

In this example, a biphasic nano-dispersed (BIND) interface to integrate the soft, rigid and encapsulation components together without paste, towards a reliable stretchable hybrid electronic device was explored. Such BIND interface comprises a biphasic nanostructure on the surface, where metallic nanoparticles, such as Au, interpenetrate into self-healing polymer matrix, resulting in continuous mechanical and electrical pathways (FIG. 1B). When pressed together face-to-face, the BIND interfaces can form BIND connection in between encapsulation, soft, and rigid components, with no need for any paste. Compared to traditional connections using paste, the BIND connection shows superior electro-mechanical properties, due to its balanced ratio between polymer and metal phases. As a typical stretchable hybrid device, the in vivo neuromodulation electrode via BIND connection shows the necessity for modular design over one-piece configuration. A more complex device, 21-channel on-skin EMG electrode, was successfully integrated, which exhibits high robustness and resistance against mechanical interference owing to the high signal fidelity and environmental adaptability of the BIND connection.

### Example 2A: Methods—Preparation of BIND Interface and Connection

BIND interface: SEBS solution (Tuftec™ H1221, 13 wt. % in toluene) of 19 ml was poured into glass mold with lid (diameter 150 mm), and evaporated in fume hood at room temperature (e.g. 25 to 40° C.) for 3 days. The evaporation speed was controlled to be slow by opening a small slit for lid. The as-prepared SEBS film (thickness ˜100 μm) was gently peeled off from the mold, and fixed on supporting filter paper by Kapton tape. The interpenetrating gold (or silver) nanoparticles were deposited using a tungsten boat (2.00 inch Diameter and 0.125 inch Thick 99.95%, Kurt J. Lesker) and a vacuum thermal evaporator (Nano 36, Kurt J. Lesker, chamber temperature 23° C., pressure ˜1*10⁻⁶ Torr, sample holder rotation 20%). The Au/Ag source was purchased from Kurt J. Lesker with purity of 99.99%. Customized mask was used when needed. For mechanical test, sample sizes were cut and standardized to 5 mm (width)*30 mm (length), and the resistance measuring distance was 20 mm.

As for a control interface, PDMS/Au thin film was prepared by spin-coating PDMS precursor (SYLGARD™ 184, Dow Inc., curing ratio 10:1) on fluorinated Si/SiOₓ substrate at 600 rpm for 60 s, and cured in 60° C. oven for more than 12 hrs. Then chromium (Cr) (5 nm, 0.5 Å/s) and Au (45 nm, 0.5 Å/s) was deposited on as-prepared PDMS film (thickness ˜100 μm), using the same vacuum thermal evaporator parameters. SiOₓ denotes for oxides of silicon.

BIND connection in between soft-soft components: BIND connection was synthesized by pressing two BIND interfaces (5 mm width and 30 mm length) face-to-face, with an overlapping area of 5 mm (width)*10 mm (length). The overlapping area was pressed via a 500 g weight for 1 hr. For mechanical and electrical test, the sample sizes were cut and standardized to 5 mm (width)*30 mm (length), and the resistance measuring distance was 20 mm. A control PDMS/Au connection was synthesized by using sandwiched commercial conductive paste/tape to bind PDMS/Au interface together, with the same sizes. The commercial conductive paste/tape are: ACF tape (3M™ ECATT 9703), copper (Cu) tape (3M Scotch™ 77802), carbon tape (PELCO Image Tabs™), and 4 types of silver paste (MG Chemicals 8331, Electrolube fast silver paste, EPO-TEK H27D silver epoxy, EPO-TEK H20E silver epoxy). The term “component” may also be referred to as “module” in the present disclosure.

BIND connection in between soft-rigid components: The rigid substrates (PI, PET, and glass) were coated with SAM (self-assembled monolayer) to enhance the adhesion with SEBS. The rigid substrates were treated with oxygen plasma to enhance hydrophilicity (PICO diener, pressure 5 mbar, power 80%, time 2 mins), and immersed into trichloro(phenyl)silane solution (Sigma 440108, 0.1 vol. % in toluene) for 0.5 hr, then rinsed with toluene, chloroform and ethanol in turn, before drying via nitrogen blow. Then the rigid substrates were dip-coated with SEBS solution (13 wt. % in toluene). The dip-coated area was −6 mm length from the end, which was defined by using scotch tape as mask during dip-coating. The rigid substrates with SEBS were put on 60° C. hot plate in fume hood for 0.5 hr to evaporate the toluene, and then put in 200° C. oven for 15 mins to avoid lifted edge of SEBS. Then, gold nanoparticles were deposited using thermal deposition, with the same parameters as before, resulting in rigid component with BIND interface. To synthesize BIND connection in between soft-rigid components, a rigid component with BIND interface and another BIND interface were pressed together face-to-face, with overlapping area of 5 mm (width)*5 mm (length). The overlapping area was pressed using 500 g weight for 1 hr. For mechanical and electrical test, the sample sizes were cut and standardized to 5 mm (width)*25 mm (length), and the resistance measuring distance was 15 mm. As a control, PDMS/Au connection was synthesized, using sandwiched commercial conductive paste/tape to bind rigid substrate with Au coating and PDMS/Au interface together, with the same sizes.

BIND connection in between soft-encapsulation components: For the peeling force test, the encapsulation layer was a SEBS thin film (thickness of ˜100 μm), and the BIND interface was synthesized as before. The BIND interface was covered with encapsulation layer on top, and pressed with 0.1 MPa pressure for 1 hr. As for the control, the PDMS/Au interface was covered with PDMS encapsulation layer (thickness of ˜100 μm), by spin coating PDMS prepolymer (at 10:1 curing ratio, 600 rpm, 60 s) on top and cured at 60° C. oven for more than 12 hrs. For practical electrode encapsulation with precisely exposed pads, much thinner encapsulation film was used (thickness of ˜300 nm). Here the Si/SiOₓ wafer was treated with oxygen plasma to enhance hydrophilicity (pressure 5 mbar, power 80%, time 2 mins), before spin coating (600 rpm, 60 s) water-soluble sacrificial layer (poly(4-styrenesulfonic acid) solution, Sigma 561223). Then dilute SEBS solution (3 wt. % in toluene, 2000 rpm, 60 s) was spin coated, and toluene was evaporated in fume hood slowly, resulting in SEBS film of ˜ 300 nm. The as-prepared sample was stuck to a PI frame, and immersed into water to get a frame supported SEBS encapsulation layer. On BIND interface, the electrode pads were covered by filter paper with the same diameter, and then the BIND interface was covered by encapsulation layer. By removing the sandwiched filter paper, the encapsulation layer on top of pads was removed together, resulting in precise exposed pads and well-defined encapsulation.

### Example 2B: Methods—Electrical, Mechanical, and Electrochemical Characterization

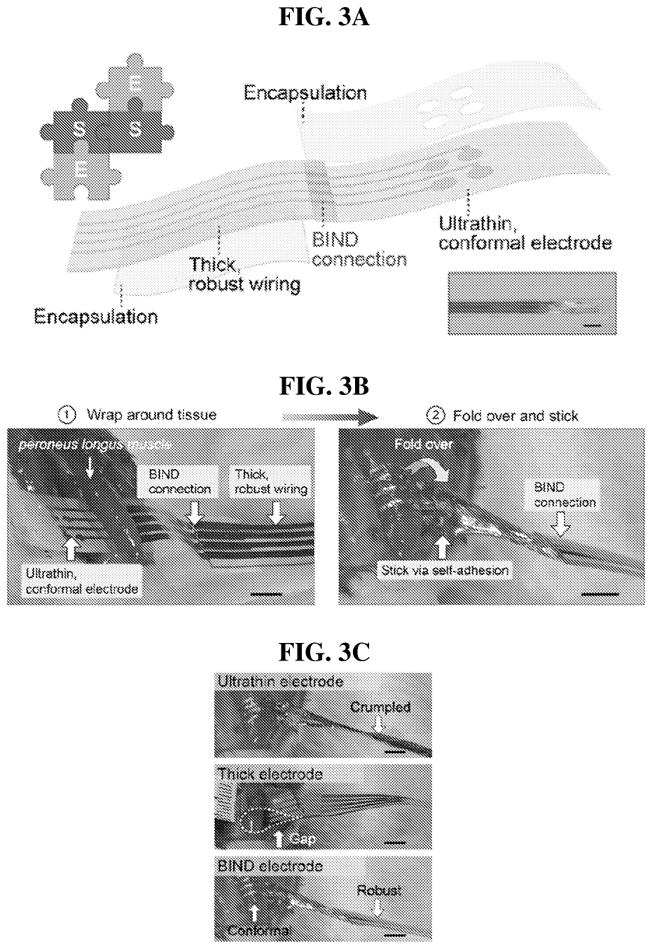

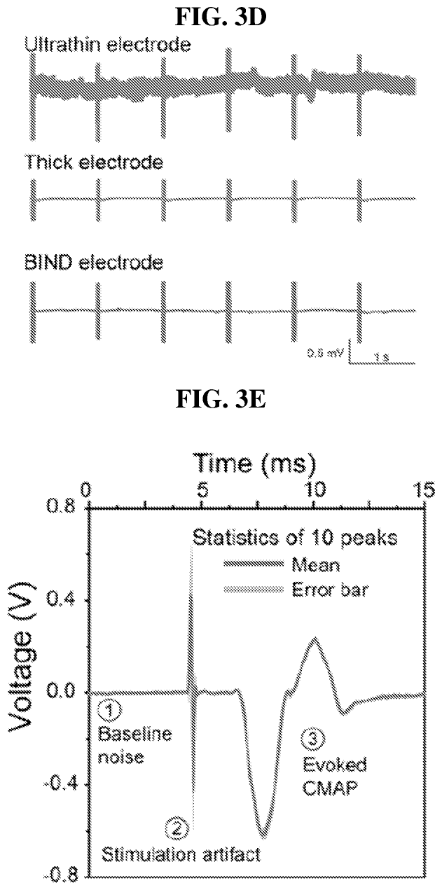

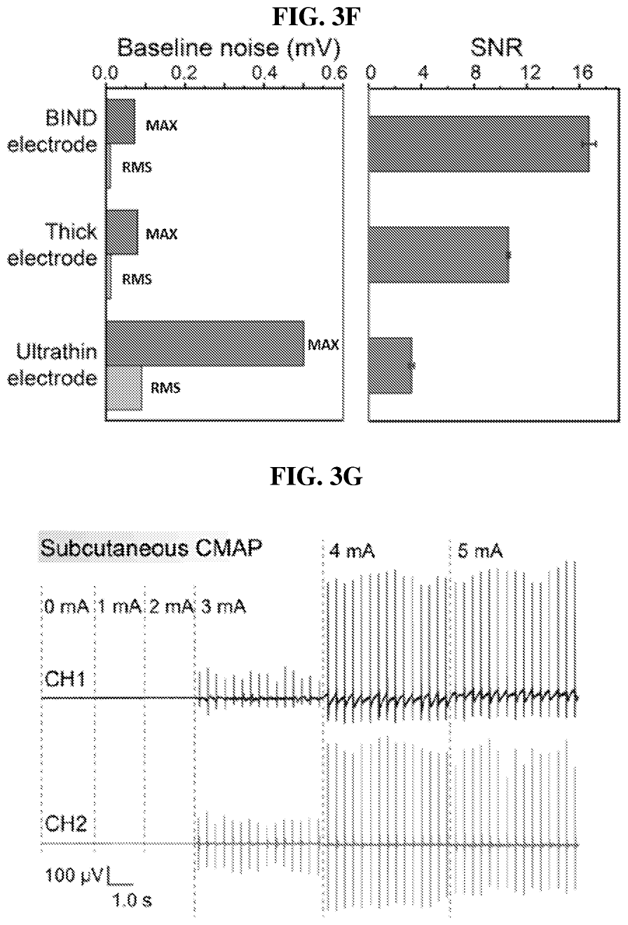

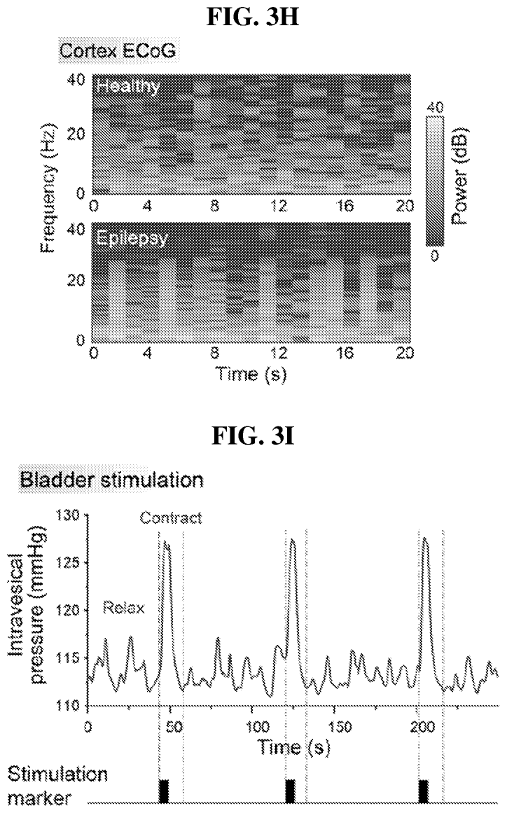

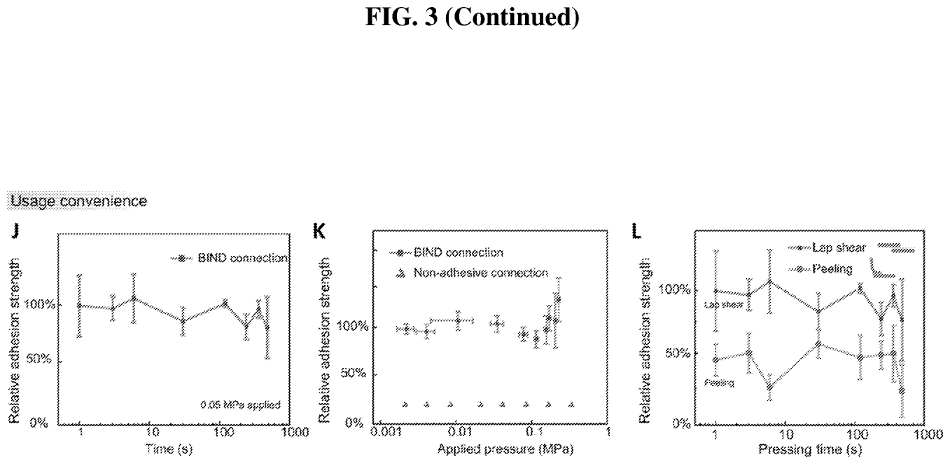

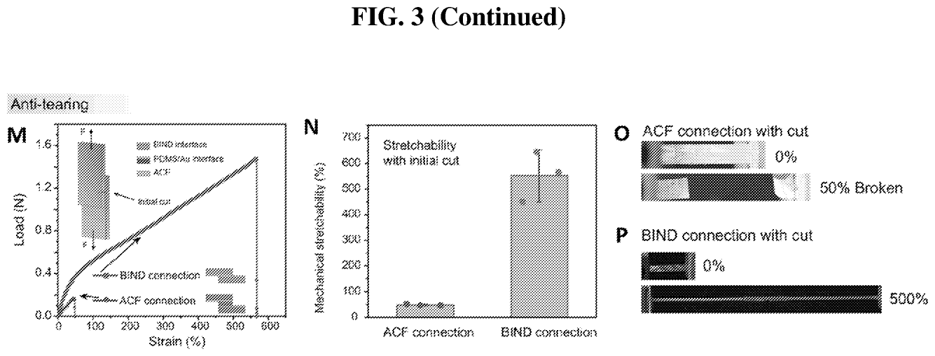

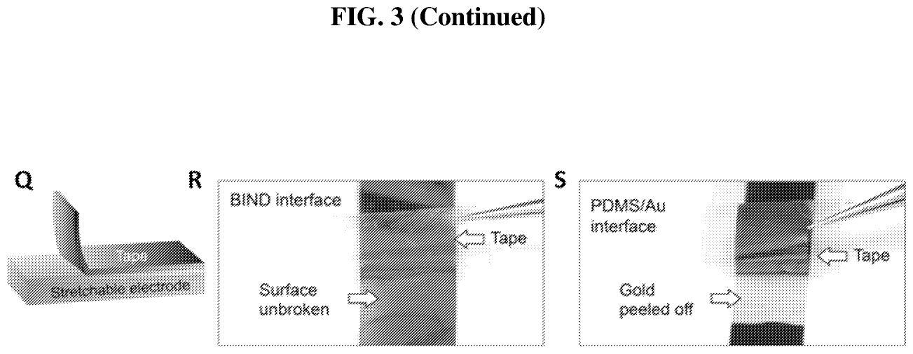

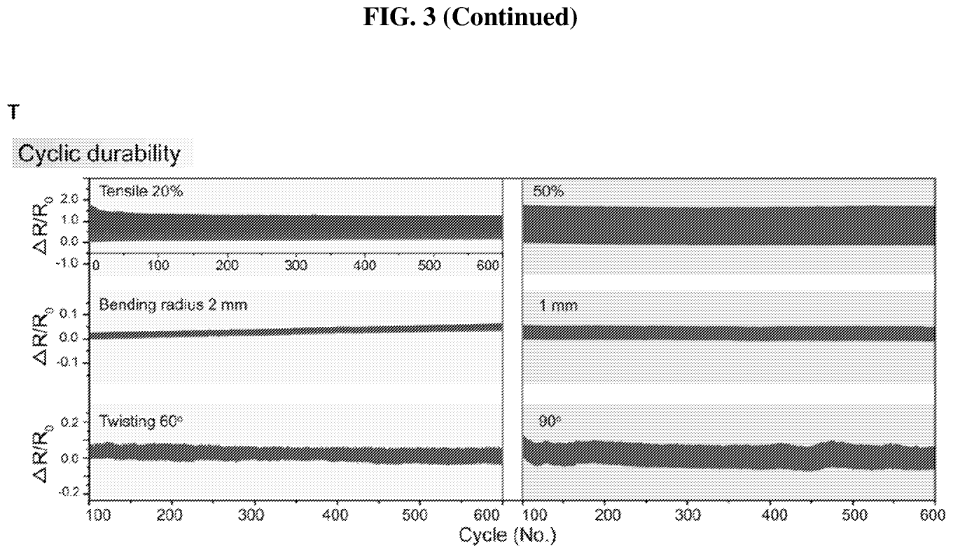

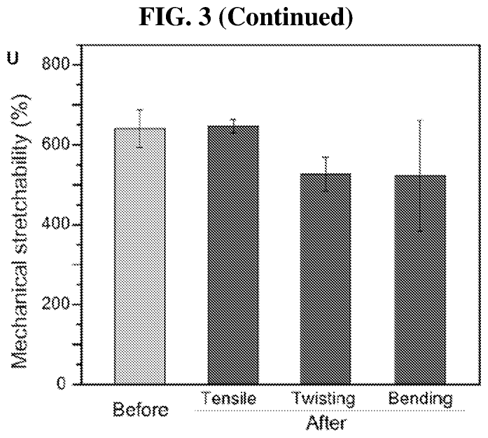

The electrical resistance was measured by semiconductor parameter analyzer (Tektronix Keithly 4200-SCS, or Keysight 34450A digital multimeter), using liquid metal EGaIn (Sigma 495425) to make contact. To simultaneously obtain electrical and mechanical results, mechanical strain was applied by a mechanical tester (MTS Systems C42, or Thorlabs LTS150/M). The electrical performance was not stable in the first few stretching/releasing cycles due to microcrack propagation (FIG. 3T), so all the electrical performance was measured after it reached stable status (usually after 100 cycles).

The sheet resistance was calculated by measuring the resistance between opposite sides of a square-sized sample (0.5 cm²). For surface sheet resistance, liquid metal is applied only on the surface of the sides. For cross section sheet resistance, liquid metal is in contact with both the surface and cross section of the sides. The resistance measurement was conducted within 2 mins to avoid dissolution of the gold in liquid metal.

Electrochemical impedance was measured using electrochemical workstation (ZAHNER ZENNIUM) from 1 Hz to 105 Hz. The BIND interface sample has original area of 0.5 cm (width)*1.6 cm (length), and its two ends were fixed on glass slide by tape. The strain was applied by controlling the distance of the tape. The sample was immersed in PBS buffer solution, with Pt as counter electrode and Ag/AgCl as reference electrode.

### Example 2C: Methods—AFM Characterization and Mechanical Analysis of Soft-Soft BIND Connection

To prepare cross section, the BIND connection was cut by glass blade using LEICA EM UC7 Ultramicrotome, under environment of −70° C. to obtain a flat cross-section surface. Specially, the non-adhesive interface cannot bind to each other, so epoxy was applied on it to form a connection. The image of this connection was segmented, to obtain separated cross section of non-adhesive interface. For AFM imaging, Bruker PeakForce™ QNM™ mode was used to simultaneously generate height, adhesion, and modulus images with quantitative data. Besides, Bruker PeakForce™ TUNA™ mode was used to generate conductivity mapping, where contact current is the average current when tip is in contact with the surface.

To investigate why BIND connection has high electrical stretchability, finite element analysis (FEA) assisted mechanical analysis was employed. ABAQUS commercial software (Dassault Systems) was used for two-dimensional FEA, to analyse the full deformation mechanics and strain distribution in connections, under uniaxial loads.

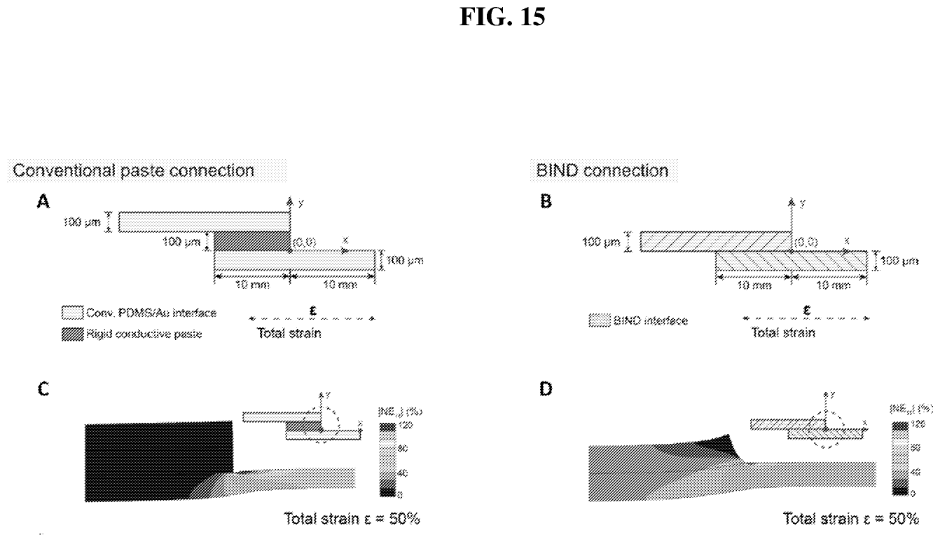

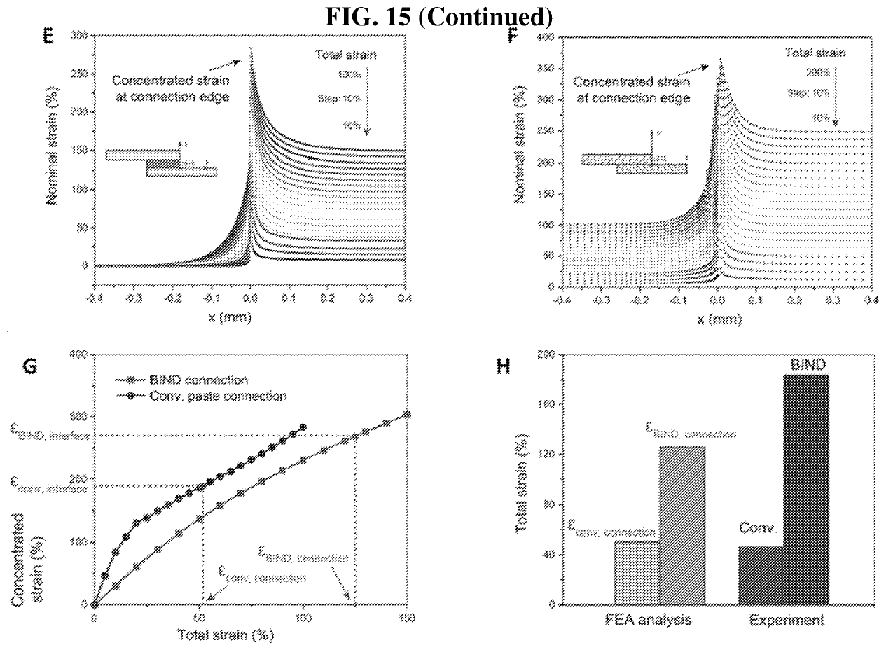

For traditional PDMS/Au interface and BIND interface, Arruda-Boyce model and Yeoh model was adopted as constitutive model, respectively. Both constitutive models employed experimentally measured non-linear stress-strain curves as input. Silver-epoxy was adopted as adhesive material, with mass density of 2 g/cm³, Young's modulus of 5 GPa, and Poisson's ratio of 0.38. Four-node plane stress quadrilateral elements were used and refined meshes were adopted to ensure the accuracy. The connection dimensions in FEA are the same as those in experimental study (interface: 20 mm length*100 μm thickness; overlapping length: 10 mm) (FIGS. 15A and 15B).

The nominal strain colour map reveals the strain distribution of conventional connection and BIND connection, under 50% uniaxial total strain (FIGS. 15C and 15D). Though both connections have concentrated strain at connection edge, the BIND connection shows more uniform strain distribution. The strain distribution under different total strain was further analysed, where BIND connection always shows wider strain distribution and lower concentrated strain (FIGS. 15E and 15F).

The maximum concentrated strain was extracted as a function of total strain of the whole connection (FIG. 15G). Compared to a traditional one, the BIND connection exhibits lower concentrated strain under the same total strain, due to the absence of rigid paste. The sufficient condition for connection to maintain conductivity is that, the local concentrated strain cannot exceed the strain tolerance of single interface, otherwise the adherend failure occurs. Here the strain tolerance of single interface can be represented as their electrical stretchability, beyond which the interface loses electrical conductivity. These values were obtained from experiments (conventional interface: ε_{conv,interface}=188%; BIND interface: ε_{BIND,interface}=269%). Therefore, from the curves of concentrated strain, the maximum total strain in connection can be deduced (conventional connection: ε_{conv,connection}=50%; BIND interface ε_{BIND,connection}=126%).

This FEA-assisted mechanical analysis shows that, compared to conventional connection, the BIND connection can endure higher total strain, due to its attenuated strain concentration. This tendency is consistent with experimental results (electrical stretchability of <46% for paste-connected connections, and >180% for BIND connection) (FIG. 15H).

### Example 2D: Methods—Auger Electron Spectroscopy

Auger electron spectroscopy (AES) measurement was performed on an Auger microprobe (JEOL JAMP-7830F) equipped with a field-emission electron gun and a hemispherical analyzer. Both secondary electron imaging (SEI) and AES were conducted with a primary electron beam having an accelerating voltage of 10 keV and a probe current of 10 nA. The analysis spot was approximately 10 μm in diameter, and the sample was tilted at 30° throughout the analysis. For Auger depth profiling, floating micro-ion etching device (FMIED) generating an ion beam of 1 keV Ar⁺ was used to sputter the sample over an area of 1.5*1.5 mm². Each cycle in the depth profiling records the spectra at an etching rate of 10 s/cycle. The spectral range for C KLL and Au MNN was 234-292 eV and 2083-2113 eV, respectively, collected with a step size of 1 eV and dwell time of 100 ms.

To examine the distribution of the Au nanoparticles in the SEBS polymer, Auger depth profiling was performed by monitoring the evolution of Au MNN and C KLL Auger transitions as a function of etch cycle. Here reactive ion etching was used to expose the inner layers.

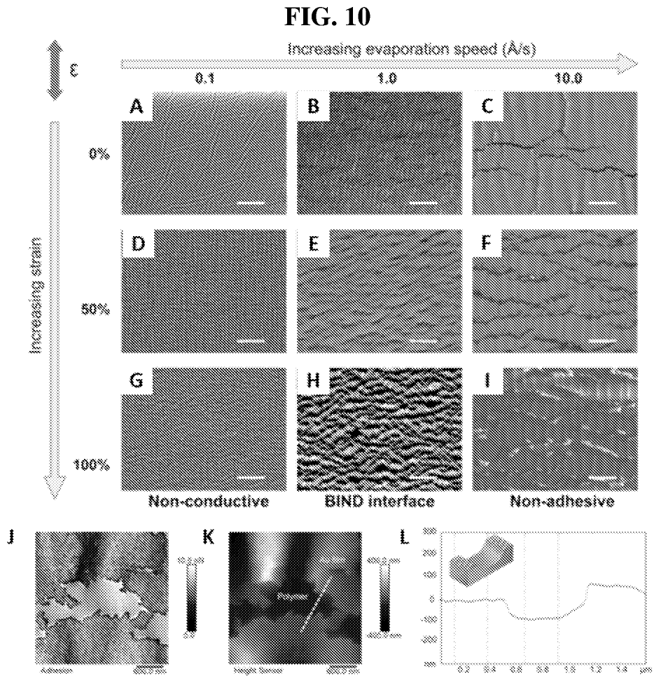

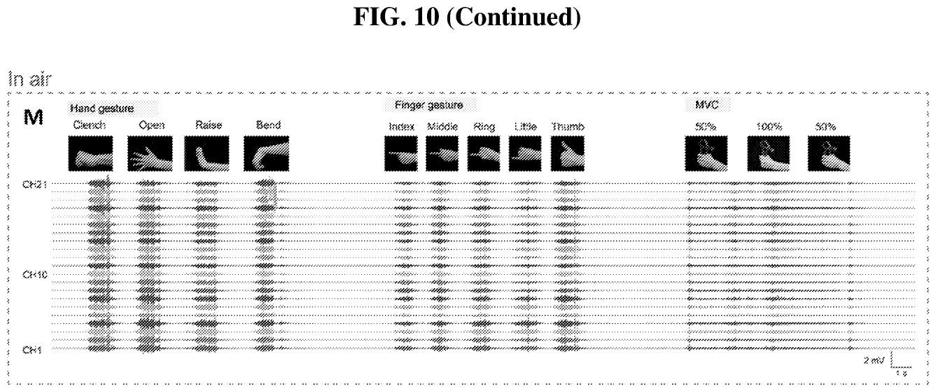

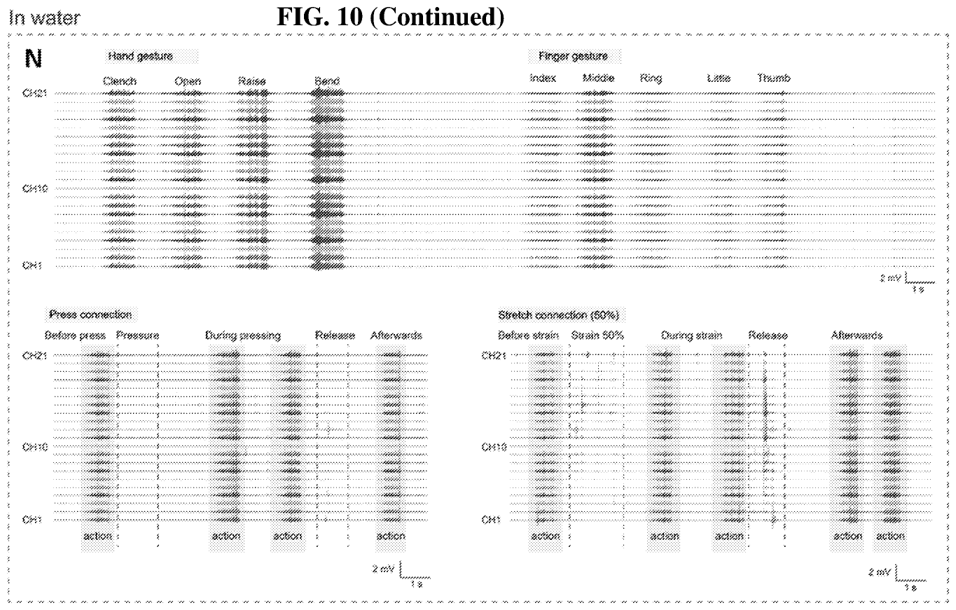

Due to the lack of structural rigidity of the samples, it is important that the sample loading was handled with care and that the samples were inspected for damage prior to the measurement. As shown in FIG. 10A to 10C, the site at which the depth profiling was performed has typical morphology, consistent with FIG. 10A to 10C.

The typical original and differentiated survey spectra shows the presence of two elements, C and Au, which corresponds to polymer and gold nanoparticles, respectively (FIG. 10D). But here the C KLL and Au NVV peaks have a significant spectral overlap, so the contribution of the latter needs to be considered, and there appears a need to deconvolute the Au NVV signal from C KLL signal. For this purpose, a control sample was prepared, i.e. Au on Si wafer. It was synthesized by thermal deposition of Au on Si wafer (the same batch as BIND interface). Spectral deconvolution was used to correct the C KLL signal, while the original C signal was noted as C (total), and the corrected C KLL signal was noted as C (real component) (FIG. 10E). Afterwards the depth profile was calculated, which is the atomic concentration as a function of depth, showing nearly 100 at. % Au and 0% at. C (FIG. 10F). This concentration is consistent with the outcome for the synthesize process of Au on Si wafer. The same deconvolution method was also applied for the other samples, including non-conductive interface, BIND interface, and non-adhesive interface. In depth profile (FIG. 10F), observably adventitious carbon significantly contributed to the C KLL signal of 0^{th} etching cycle, which scanned the top layer before any reactive ion etching. Thus, the 0^{th} etching cycle was considered as containing adventitious carbon, and not presented and calculated in further analysis.

To investigate the distribution of both polymer and metal phases in the inner structure, the differentiated scan spectra were compared and analysed (FIG. 10G to 10J). In the non-conductive interface, the C KLL signal was present in the first 10 etching cycles, then attenuated, and re-emerged at around 80^{th} cycle, where most of the Au phase disappeared (FIG. 10G). The coexistence of both C KLL and Au MNN signals suggests an interpenetrating structure of polymer and metal phases in the first 10 cycles. Similarly, the analysis of the BIND interface also shows polymer/metal interpenetration in the first few etching cycles, which is consistent with the top-viewed AFM images (FIG. 10H). On the contrary, the analysis of the non-adhesive interface exhibits nearly no carbon signal until 30^{th} etching cycles, demonstrating nearly no interpenetration of Au nanoparticles inside the polymer (FIG. 10I). Finally, the Au on Si wafer sample with obvious Au MNN signal and nearly no C KLL signal across the etching cycles further proves that in the former interfaces, the carbon signal should originate from the SEBS polymer, instead of adventitious carbon (FIG. 10J).

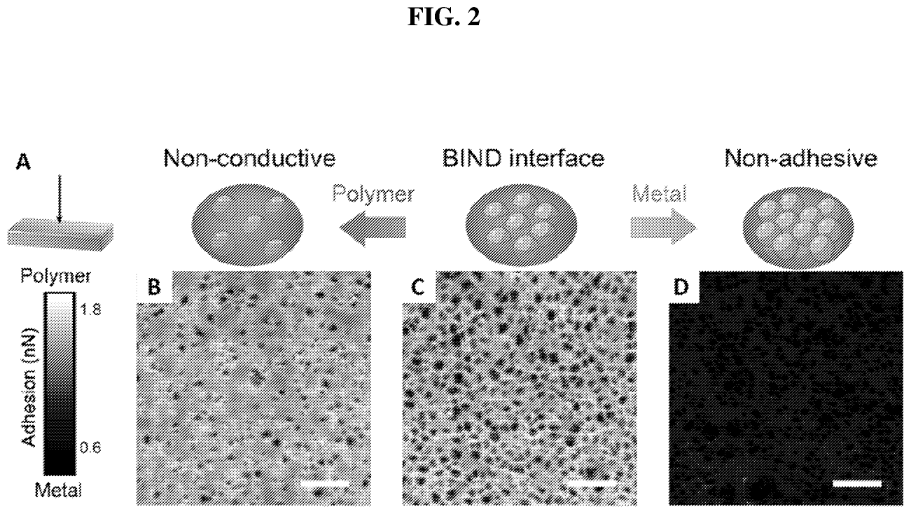

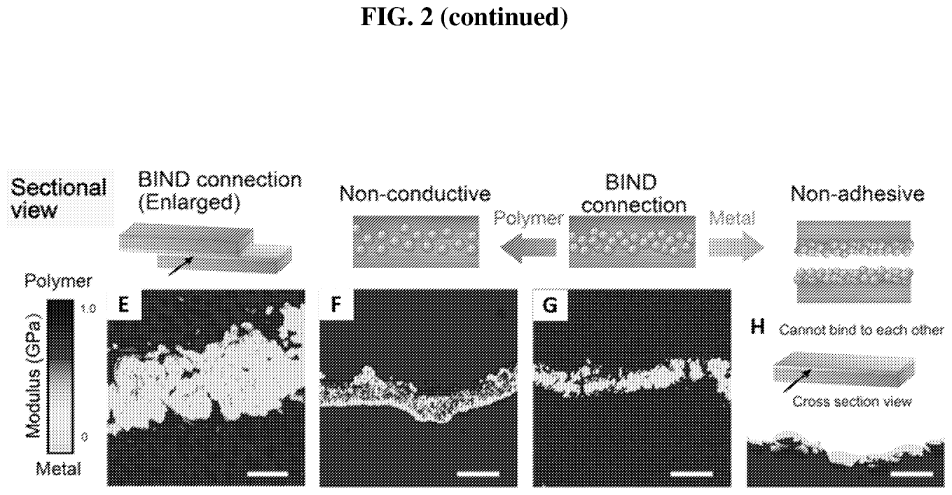

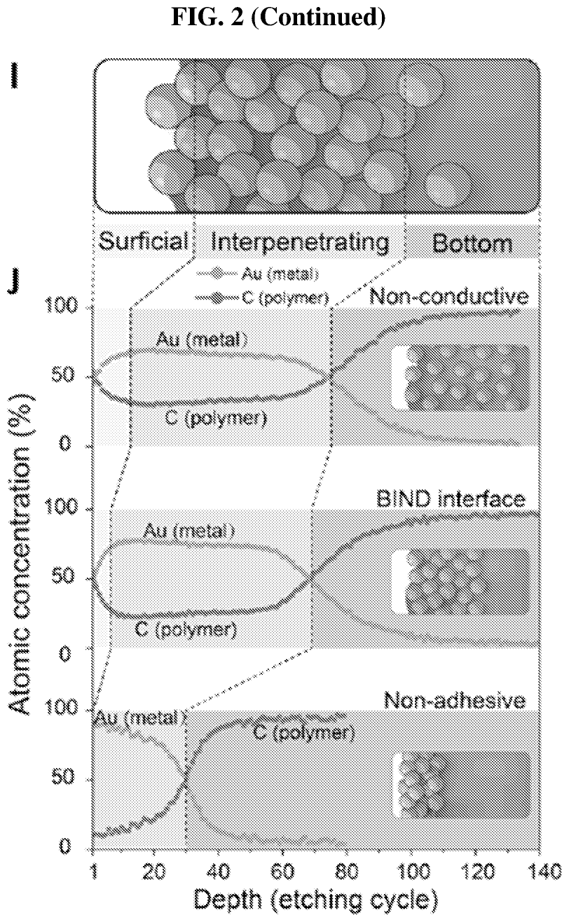

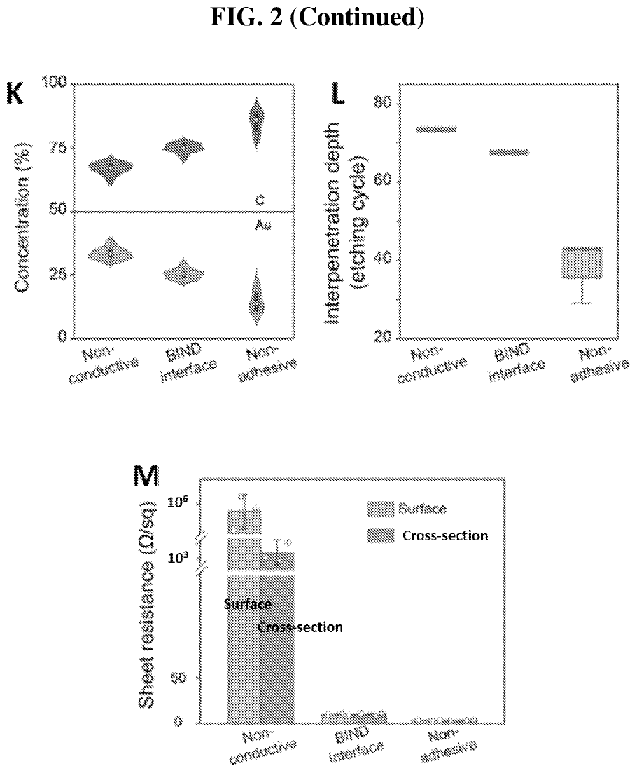

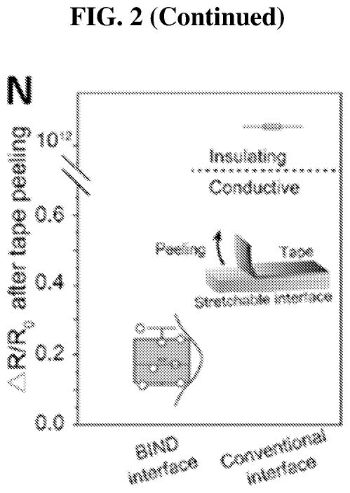

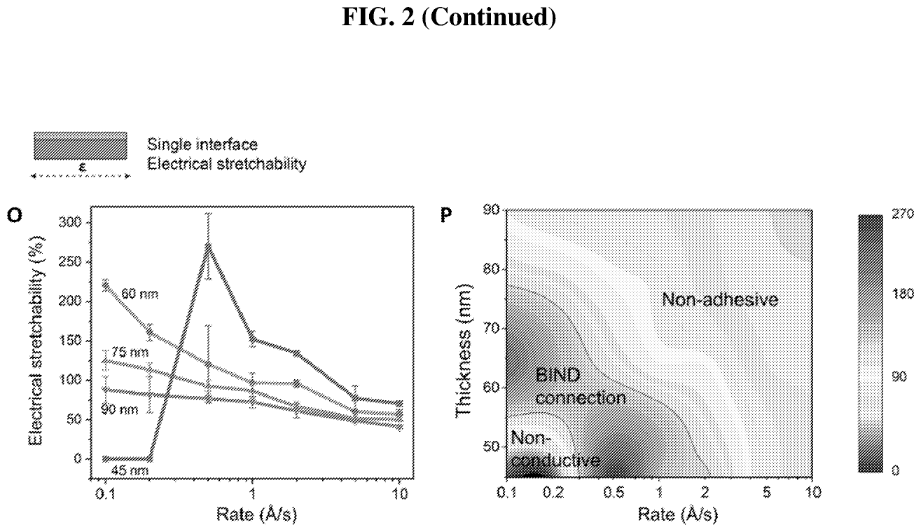

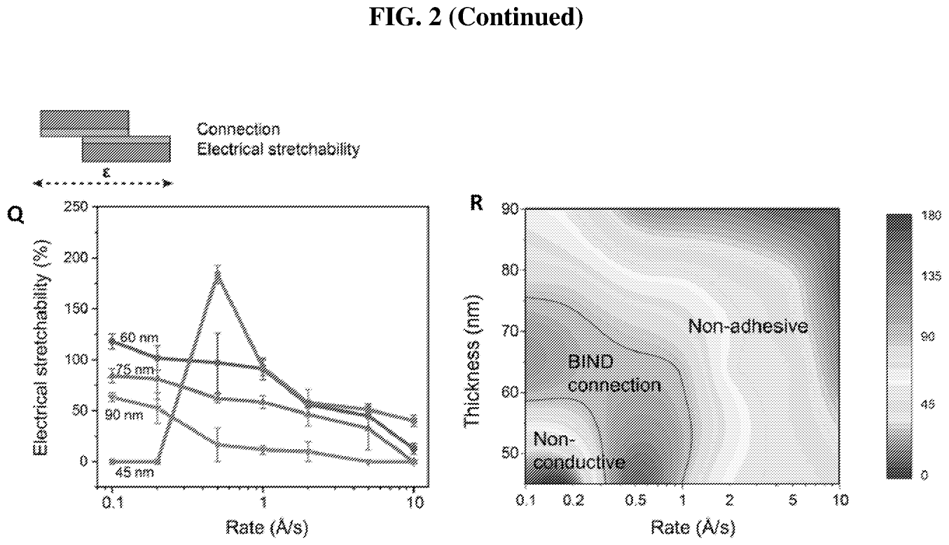

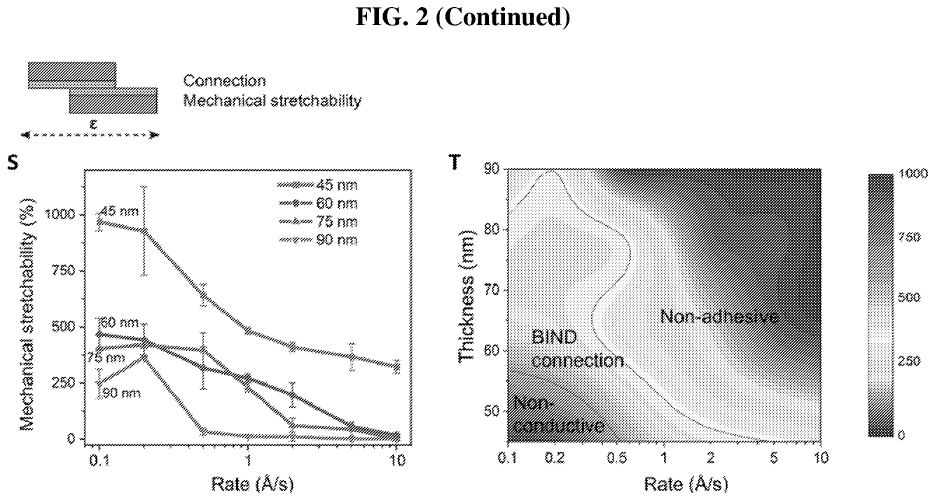

These scan spectra can be further transformed to depth profiles, which are depth-dependent atomic concentrations, based on the quantitative analysis of the measured Auger signals. The peak-to-peak height of element X, I_{X}, in the differentiated Auger electron spectra, i.e. dN(E)/dE, can be correlated with its atomic concentration, C_{X}, based on the following expression:

$${C}_{X} = \frac{{I}_{X}}{{S}_{X}} / {\sum }_{i} \frac{{I}_{i}}{{S}_{i}}$$

where S_{X} is the relative sensitivity of element X (S_{Au}=0.127 and S_{C}=0.121 for differentiated Auger spectra with an incident electron energy of 10 keV). Using this equation, the depth-dependent atomic concentration was calculated for non-conductive interface, BIND interface and non-adhesive interface, respectively (FIG. 2J).

### Example 2E: Methods—SEM Imaging

SEM images were obtained through JEOL JSM-7800FPRIME scanning electron microscope, at acceleration voltage of 2-5 kV under secondary electron image (SEI) mode. To obtain image of samples under strain, the sample was firstly stretched to 50% and 100% strain, then fixed with tape at two end. Only the middle of the sample was used for imaging.

### Example 2F: Fabrication of In Vivo Neuromodulation Electrode

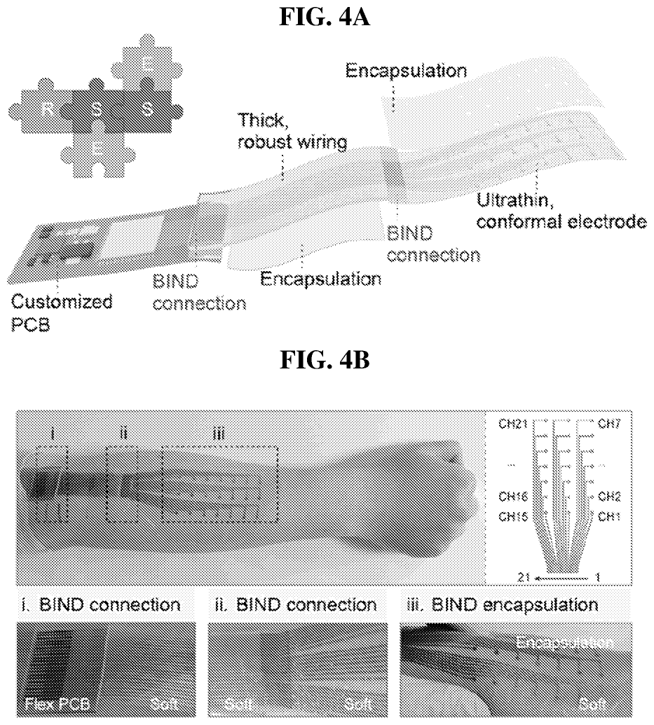

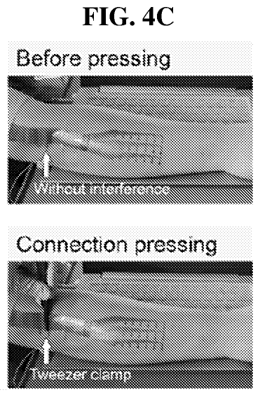

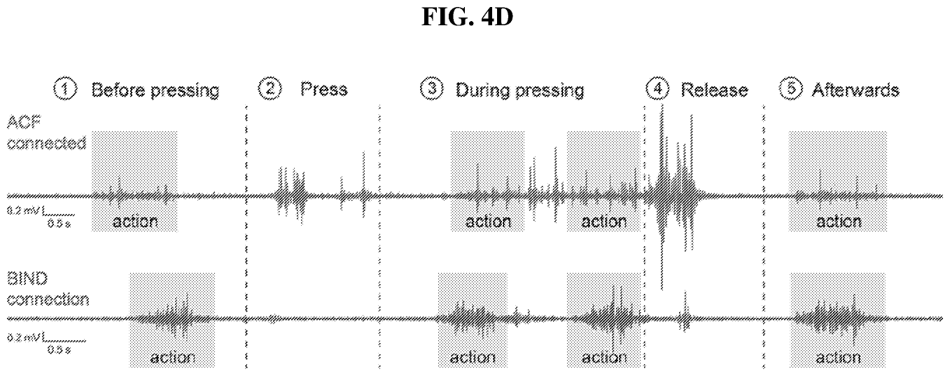

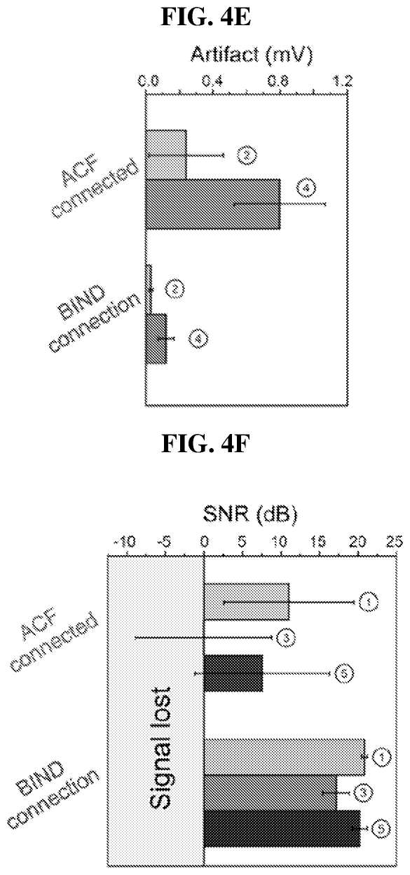

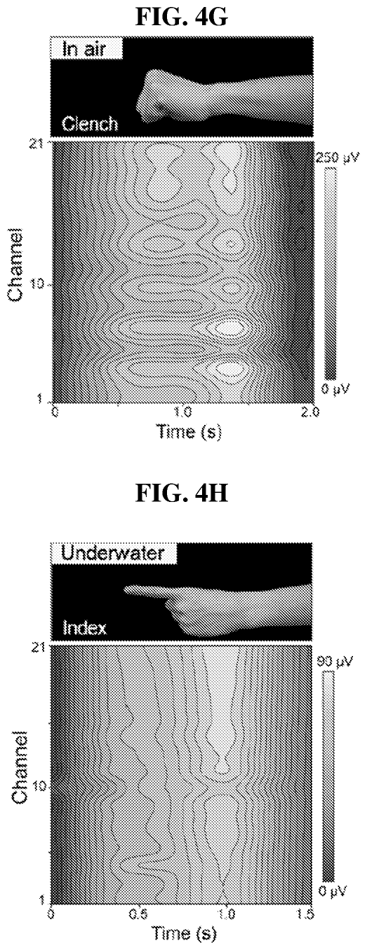

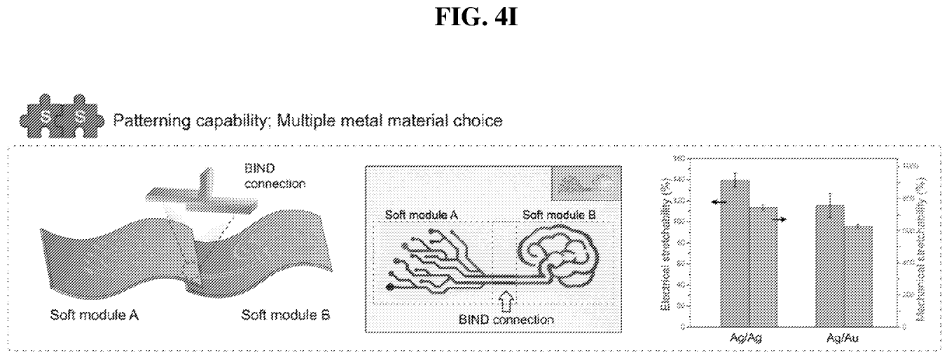

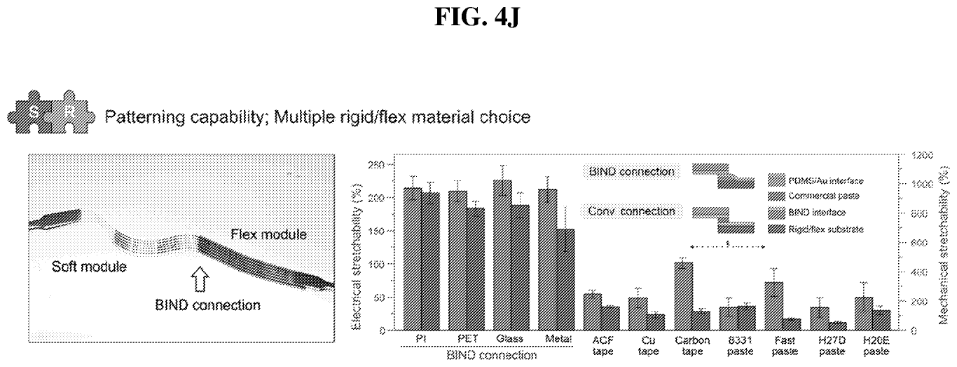

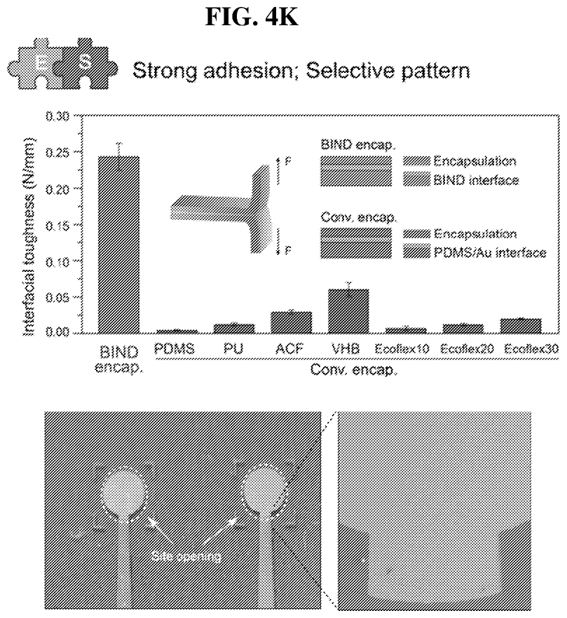

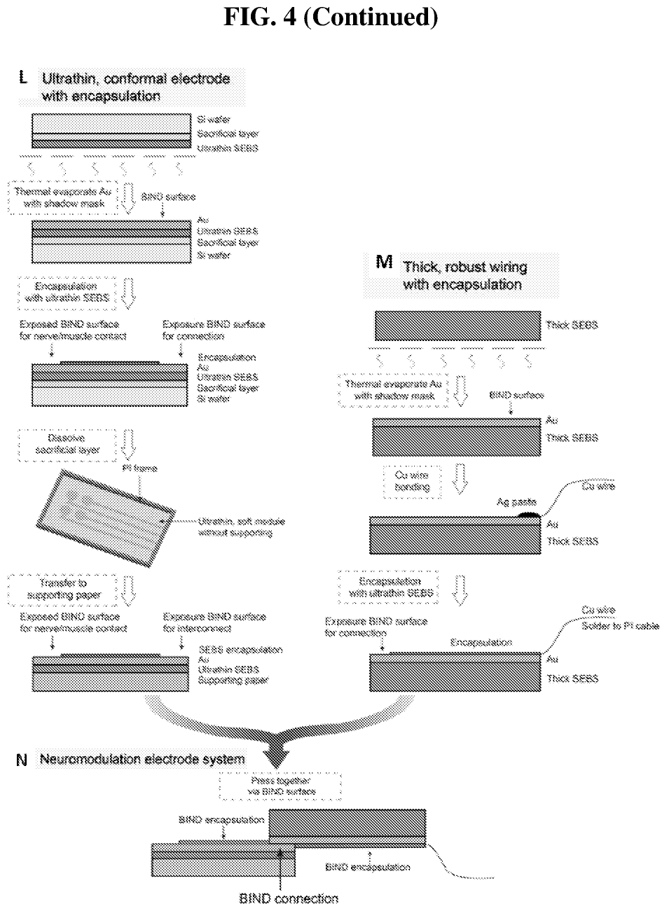

The in vivo neuromodulation electrode consists of ultrathin and conformal electrode with encapsulation, and thick and robust wiring with encapsulation. Each part was fabricated separately, and assembled via BIND connection (FIG. 4L to 4N).

Ultrathin, conformal electrode with encapsulation: Firstly, an ultrathin SEBS substrate (thickness ˜2-4 μm) was synthesized. The Si wafer was treated with oxygen plasma to enhance hydrophilicity (pressure 5 mbar, power 80%, time 2 mins), before spin coating (600 rpm, 60 s) water-soluble sacrificial layer (poly(4-styrenesulfonic acid) solution, Sigma 561223). Then dilute SEBS solution (5 wt. % in toluene) was spin coated (1000 rpm, 60 s), and the toluene was evaporated in fume hood slowly, resulting in ultrathin SEBS film of ˜2-4 μm. Secondly, the as-prepared substrate was deposited with Au (45 nm, 0.5 Å/s), using thermal evaporation and customized mask, via procedure described before. Here, an ultrathin, conformal electrode with desired pattern was obtained. Thirdly, encapsulation layer of 300 nm was attached on top of the electrode with exposed area, using procedures described before. Finally, the whole electrode with encapsulation was stuck to a PI frame, and immersed into water, before transferred to a flexible supporting paper.

Thick, robust wiring with encapsulation: The thick, robust wiring was synthesized by depositing Au (45 nm, 0.5 Å/s) on SEBS film (˜150 μm), using thermal evaporation and customized mask, via procedure described before. Then the wiring was covered with encapsulation layer (thickness ˜300 nm), except two ends to expose BIND interface for further connection.

As a control to show advantages of modular design, two types of electrodes were fabricated: the ultrathin electrode, and the thick electrode. The two electrodes were fabricated by depositing Au (45 nm, 0.5 Å/s) on ultrathin SEBS film (˜2-4 μm) and thick SEBS film (˜150 μm), separately, via thermal evaporation procedure described before.

### Example 2G: Methods—In Vivo Experiment

All in vivo experiments except bladder experiments were performed in Shenzhen Institutes of Advanced Technology, Chinese Academy of Sciences, which were approved and adhered to guidelines of its Institutional Animal Care and Use Committee. Experimental subjects (healthy, adult male Sprague-Dawley rats, ˜250-300 g) was adopted, and housed within appropriate temperature (23-25° C.) and humidity (50-70%). The animal was anaesthetized by injecting sodium pentobarbitone (density 2%, dose 0.3 ml/100 g) into the intraperitoneal space, and checked for the depth of anesthesia. The skin on area of interest was shaved and depilated, and the body temperature of the rat was maintained with an electrical heat pad at 37° C.

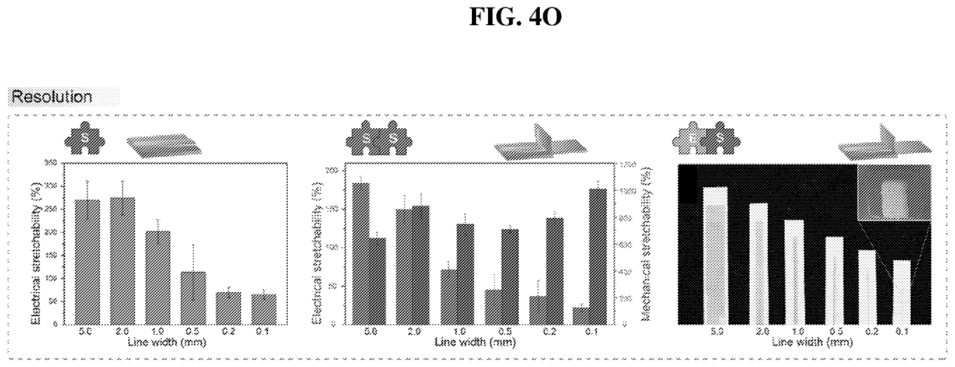

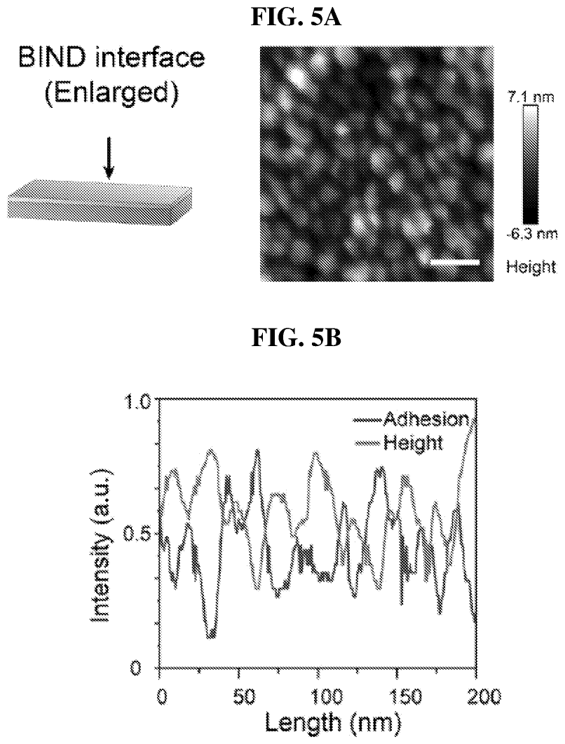

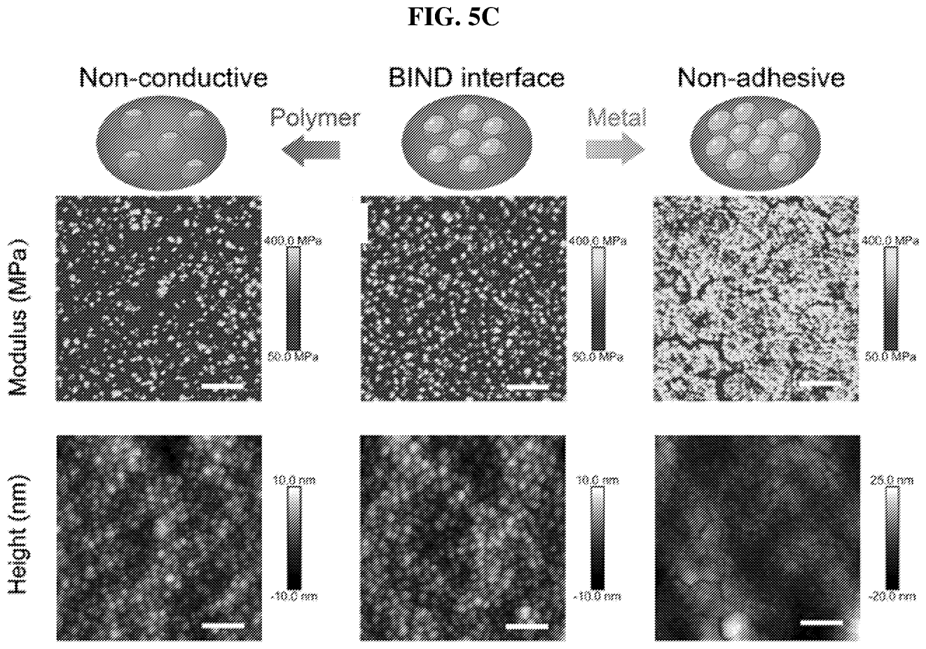

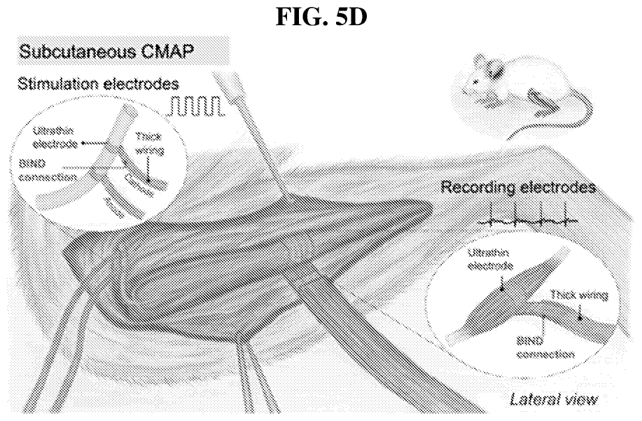

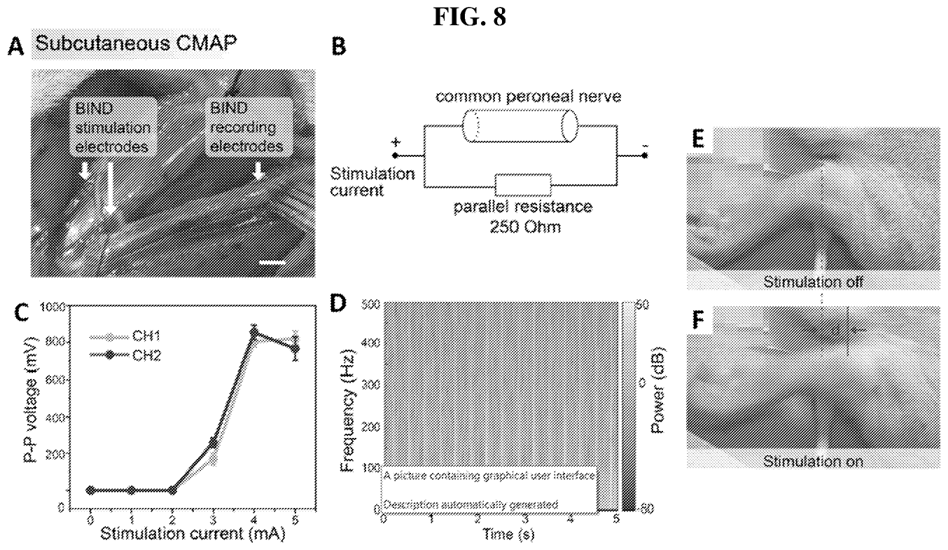

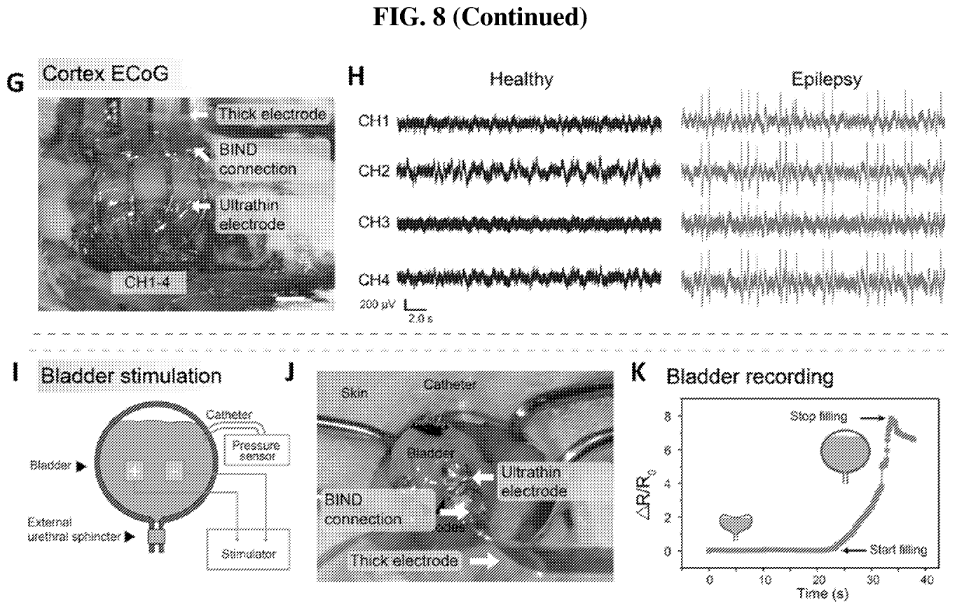

Stimulation of sciatic nerve and peroneal nerve: In simultaneous CMAP stimulation and recording experiment, a BIND electrode was used to stimulate peroneal nerve via suture-free procedure (FIG. 5D). In this procedure, biceps femoris was bisected to uncover and isolate peroneal nerve, and the area was irrigated with sterile saline to ensure clear view and good electrical contact. The ultrathin module of BIND electrode with supporting paper was firstly inserted into the gap in between nerve and underneath muscle, then it was connected to the thick module. Then the ultrathin module was wrapped around the nerve, and the tail was stuck via self-adhesion before removing supporting paper. The procedure was repeated two times for cathode and anode. The stimulation pulse was applied via Neurotrac continence electrical stimulator (monophasic pulse, width 150 μs, and frequency 3 Hz), and the stimulation current was applied to the nerve with a 250 ohm parallel resistance (FIG. 8B).

In sciatic stimulation experiment, the BIND electrode was used to stimulate sciatic nerve, with the same process and parameter as aforementioned.

In CMAP recording comparison experiment, 2-channel polyimide electrode (anode and cathode) was used to stimulate peroneal nerve, via Neurotrac continence electrical stimulator (monophasic pulse, width 100 μs, and frequency 1 Hz).

Subcutaneous CMAP recording on peroneus longus muscle: Incision was made on skin for clear visualization, and the peroneus longus muscle was isolated, with sterile saline irrigation. Similar to nerve stimulation, BIND electrode was wrapped on peroneus longus muscle via suture-free wrapping procedure. In CMAP recording comparison experiment, the control electrodes (ultrathin electrode and thick electrode) were wrapped around peroneus longus muscle using the same procedure, and reference electrode was placed on rat sole. In CMAP simultaneous simulation and recording experiment, channels in BIND electrode was used for both working and reference electrodes.

ECoG recording: The anaesthetized rat was positioned on a stereotaxic frame, then a longitudinal incision was performed to expose the skull surface, and a round parietal craniotomy was made with a surgical drill. The BIND electrode was gently placed on the surface of cerebral cortex to record ECoG signals, where the ultrathin module directly contact with the cortex. The BIND electrode was connected to external equipment (Bluetooth video electroencephalograph system, Nation Inc., Shanghai, China) by clamping the equipment cable. After half an hour of stable recording, penicillin (dose of 2 million units/kg of body weight) was injected to induce seizure, and the corresponding ECoG signal was recorded after about half an hour.

Bladder experiments: Bladder experiments were performed in the N.1 Institute of Health, Singapore, where animal care and use procedures were approved by the Institutional Animal Care and Use Committee (IACUC) of the National University of Singapore. The experimental subjects were adult female Sprague-Dawley rats (˜220-300 g), and were anaesthetized by injecting a mixture (0.2 ml/100 g) of ketamine (37.5 mg/ml) and xylazine (5 mg/ml) into the intraperitoneal space for induction, and checked for the depth of anesthesia regularly with supplementary dose of 0.1 ml/100 g injected intraperitoneally for maintenance. After shaving hair on lower abdominal area, incisions were made on the skin and underlying subcutaneous tissue, muscle, and peritoneum using aseptic techniques. Fat and connective tissues were gently removed or pushed aside, in order to make the bladder wall come into view. In bladder stimulation experiment, BIND electrode was sutured on bladder wall, while the ultrathin part directly contacted the bladder wall, and thick part provided robust wiring (FIG. 8I to 8J). A catheter (C30PU-RCA1302, Instech Laboratories Inc., PA, USA) was inserted into the bladder via a small cut at the bladder dome and secured with a 4-0 silk suture, which enabled injection of saline to fill the bladder and connection to an external pressure sensor (Transpac® IV, ICU Medical Inc., CA, USA) to detect the intravesical pressure inside the bladder. When electrical stimulation pulses (300 s biphasic pulses, 10 Hz, 3 mA) were applied to bladder wall via BIND electrode for a duration of 5 seconds, the bladder muscle contracted and resulted in increases in intravesical pressures that were measured by the external pressure sensor. In bladder sensing experiment, the BIND electrode was wrapped around the bladder wall. Normal saline was filled into bladder through the catheter, and changes in bladder size were detected by BIND electrode (FIG. 8K).

### Example 2H: Methods—Fabrication of 21-Channel On-Skin EMG Electrode

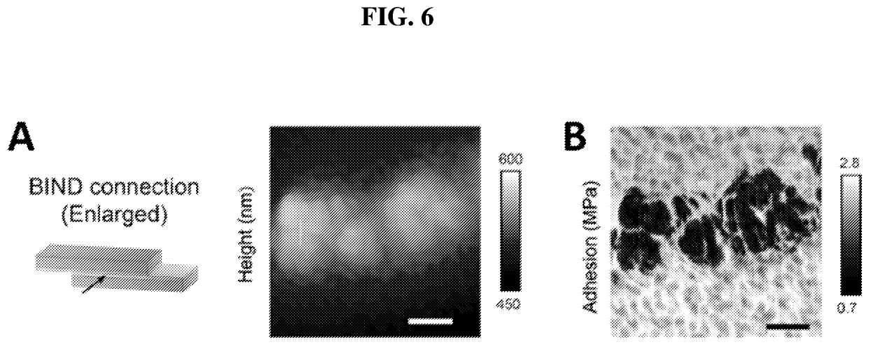

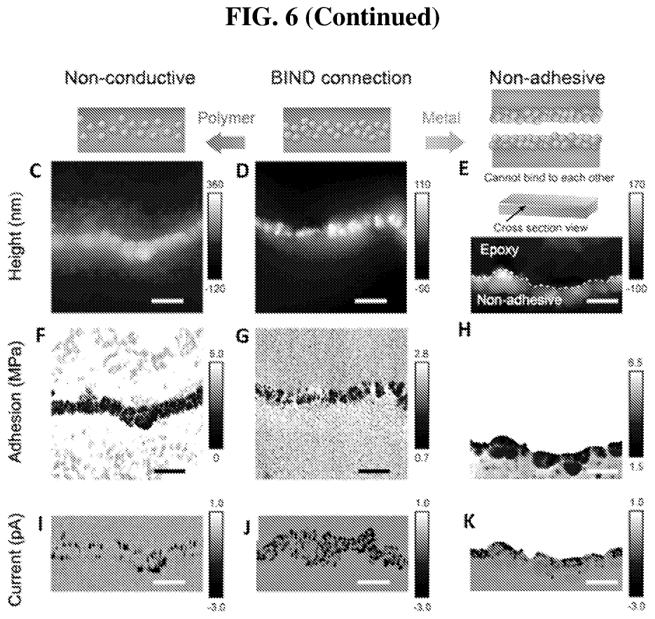

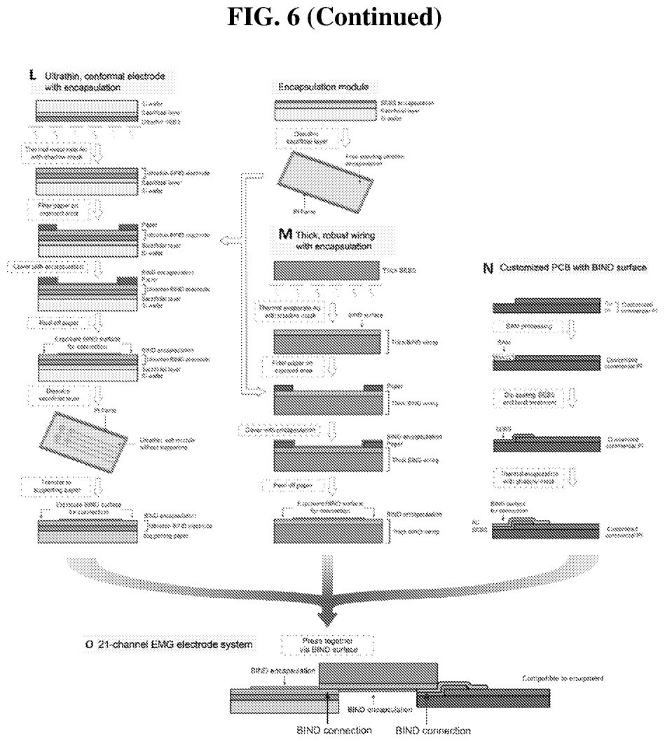

The 21-channel on-skin EMG electrode consists of: the ultrathin, conformal electrode with encapsulation, the thick, robust wiring with encapsulation, and the customized PCB with BIND interface. Each part was fabricated separately, and assembled via BIND connection (FIG. 6L to 6N). For both BIND electrode and ACF-connected electrode, the recorded signals were applied to a 50 Hz notch filter and a 10 Hz high pass filter, except that the signals under connection pressing/stretching were applied to 30 Hz high pass filter.

Ultrathin, conformal electrode with encapsulation, and thick, robust wiring with encapsulation: These two parts were synthesized via the same procedure as described in method “Fabrication for in vivo neuromodulation electrode”, i.e. example 2F above, using different customized mask for 21-channel electrode pattern.

Customized PCB with BIND interface: The commercial customized PCB (0.5 oz Cu on polyimide substrate, gold plating) was compatible to high density biopotential signal collector (SIAT, China) via cable socket. The exposed polyimide substrate at the end was coated with BIND interface, using the procedure described in method “BIND connection in between soft-rigid components”.

As control, ACF-connected electrode used the same configuration/size/pattern as BIND electrode, which also consists of ultrathin, conformal electrode with encapsulation, thick, robust wiring with encapsulation, and customized PCB. Firstly, the ultrathin, conformal electrode was prepared by depositing Cr (5 nm, 0.5 Å/s) and Au (45 nm, 0.5 Å/s) on ultrathin PDMS substrate (thickness ˜2-4 μm), using the same thermal evaporation parameters. The ultrathin PDMS substrate and encapsulation layer was prepared by spin coating diluted PDMS prepolymer (PDMS 10:1 curing ratio, 20 wt. % in hexane, 1000 rpm, 300 s) on top of fluorinated Si/SiOₓ substrate and cured at 60° C. oven for more than 12 hrs. Both the electrode and encapsulation layer were treated with oxygen plasma (power 80%, time 2 mins) to enhance the adhesion on both surfaces, before they were joined by pressing together. The electrode pads were exposed by removing sandwiched filter paper with the same diameter of the pads. Secondly, the thick, robust wiring with encapsulation was prepared on PDMS substrate (thickness ˜100 μm), using the same procedure as in ultrathin PDMS electrode. Thirdly, commercial ACF tape was used to connect the ultrathin, conformal electrode, the thick, robust wiring, and the customized PCB together, in order to obtain the ACF-connected electrode.

### Example 3A: Results and Discussion—Electro-Mechanical Performance

Using the BIND interface, BIND connections can be formed in between soft, rigid and encapsulation components (FIG. 1C). The soft-soft BIND connection is formed by simply pressing two BIND interfaces against each other without additional paste, wherein an exposed polymer and metal phase at the surface contribute to mechanical and electrical binding, respectively. For soft-rigid BIND connection, BIND interface was first deposited on rigid/flexible substrate, before pressed to another soft BIND interface. Lastly, the soft-encapsulation BIND connection is formed by simply pressing an encapsulation SEBS layer on top of the BIND interface.

The soft-soft BIND connection exhibits high electrical stretchability of >180%, and mechanical stretchability of >600%, superior to that of commercial conductive pastes (FIG. 1D and FIG. 1G to 1N). Meanwhile, the small relative resistance change (4 times resistance change at 50% strain) in the BIND connection contributes to stable performance under mechanical deformation (FIG. 10). The electro-mechanical performance of BIND interface is regulated via fabrication parameters, specifically, deposition rate and thickness (FIG. 2O to 2T). Only a certain range of deposition rate (0.5-1.0 Å/s) and thickness (45-60 nm) results in BIND interface that can form soft-soft connection. Larger deposition rate and thickness results in non-adhesive interface that cannot bond to each other, while smaller rate and thickness makes non-conductive interface that is not electrically conductive at all.

Furthermore, the pressing time, pressure and peeling direction (lap shear or 90° C. peeling) has little influence on the adhesion strength of BIND connection, resulting in fast formation and high usage freedom in practical application (FIG. 3J to 3L). In a traditional connection, the commonly encountered damage results in fast break due to stress concentration, but the present BIND connection demonstrates 500% mechanical stretchability with initial cut, showing its anti-tearing property (FIG. 3M to 3P). The present BIND connection also demonstrates cyclic durability for over 500 cycles under different contortions (e.g. tensile, bending and twisting as demonstrated in FIGS. 3T and 3U). The paste-free feature of BIND connection makes it suitable for high-resolution patterning, where conventional pastes has resolution limitation (FIG. 4I (left and center images)). Also, the conformality of connection can be improved without the use of paste, beneficial for soft/ultrathin electronics device. Moreover, the metallic material choice can be expanded to Ag/Ag and Ag/Au, suitable for different device requirements (FIG. 4I right image).

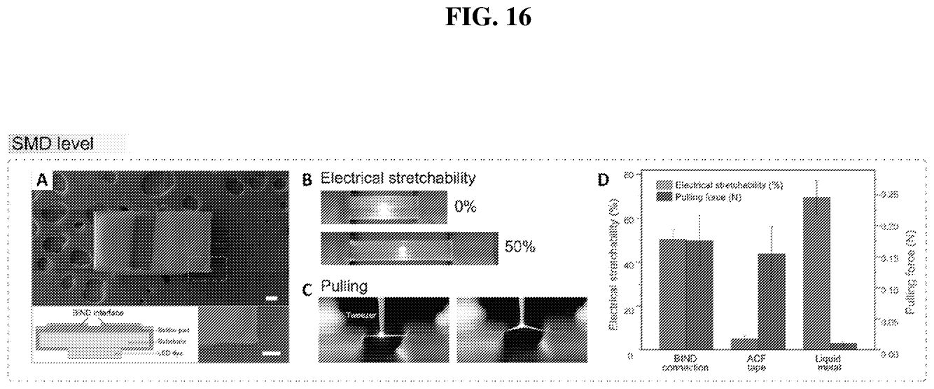

Similar to soft-soft connection, the soft-rigid BIND connection also yields high electrical stretchability and low relative resistance change, compared to ACF-connected electrodes (FIG. 1E). The feasibility for patterning enables multi-channel BIND connection, and the material choice of rigid/flexible component can be further expanded to polyimide (PI), polyethylene terephthalate (PET) and glass substrate (FIG. 4J). These rigid/flexible components can then be connected to other rigid electronics using mature technology, beneficial to utilize the high processing capability of Si-based microelectronics, e.g. the soft-rigid BIND connection is also compatible to surface mount devices (SMDs) such as LED (see FIG. 16). The soft-encapsulation BIND connection can be selectively patterned to expose necessary opening (see example 2A, also see FIG. 4K bottom image), such as electrophysiological electrode pads. Because the surface-exposed polymer on BIND interface provides mechanical bonding to encapsulation layer, the BIND encapsulation exhibits a peeling force of 0.437 N, 22 times larger than that of conventional PDMS encapsulation (FIG. 1F, FIG. 4K top image).

The present BIND interface can also bind encapsulation modules strongly. A SEBS encapsulation layer (100 μm thick) onto a BIND interface at 0.1 MPa for 1 hr before subjecting the soft-encapsulation BIND connection to a 180° peeling test (FIG. 4J). Interfacial toughness was defined as two times the peeling force plateau. Because the surface-exposed polymer on the BIND interface provides binding areas for the encapsulation layer, the interfacial toughness for BIND connection is 60 times larger than a traditional connection (0.24 N/mm vs. 0.004 N/mm), which consists of PDMS or other encapsulation layer onto a PDMS/Au interface (FIG. 1F). Further, the encapsulated BIND interface can be selectively patterned to expose only part of the BIND interface for applications such as electrophysiology electrode pads (see FIG. 4K bottom image). Besides, the resolution of single BIND interface, BIND connection and BIND encapsulation can achieve line width of 0.1 mm, meeting most of the need for interconnects (FIG. 4O).

To sum up, the BIND interface provides integration for soft, rigid and encapsulation components, which exhibits superior electrical and mechanical performance to current commercial pastes.

### Example 3B: Results and Discussion—Structural Analysis and Nanomechanics Investigation of BIND Interface Formation

To understand why BIND interface can adhere to each other without additional pastes, its surface and inner structure was investigated in nanoscale. Here adhesion AFM mapping was employed to provide top-view imaging of the BIND interface (FIG. 2A). The polymer and metal phases can be easily distinguished, because the former exhibits much larger adhesion force to AFM tip. The BIND interface clearly shows the co-existence of polymer and metal phase on the surface (FIGS. 5A and 5B). The ratio of polymer/metal phase in the biphasic nanostructure determines its macroscopic electro-mechanical properties (FIG. 2B to 2D, FIG. 5C). Excess of polymer phase results in highly adhesive yet non-conductive surfaces, while the lack of polymer phase deprives its self-adhesive property. Only on BIND interface, the exposed polymer and metal phases both reaches balance status. Therefore, BIND connection can be easily realized by pressing two BIND interfaces face-to-face together, while dynamic interaction between exposed self-healing polymer constructed mechanical pathway, and Ohm contact between exposed gold nanoparticles contributes to electrical pathway. The inner biphasic structure of BIND connection was explored via cross-sectional AFM mapping, showing nano-scaled interpenetrating network (FIG. 2E, FIGS. 6A and 6B). Similar to surface mapping, the inner distribution of polymer and metal phases also reflects its electro-mechanical property (FIG. 2F to 2H, FIG. 6C to 6K). The non-conductive connection exhibits much more inner polymer phase than the BIND interface, while non-adhesive interface cannot even adhere and form connection due to the concentrated metal phases on the surface.

To quantitatively investigate the inner structure of the BIND interface, Auger electron spectroscopy was conducted to sequentially reveal the inner layers layer-by-layer via exposure to reactive ion etching (FIG. 2I, 2J, example 2D, FIG. 1P to 1R). The ratio of polymer and metal phase can be determined from the carbon and gold atomic concentrations, respectively. The non-conductive interface clearly exhibits sparse and deep distribution of the metal phase, indicating insufficient electron pathway, while the non-adhesive interface has dense and stacking metal phase, with almost no polymer phase. In comparison, the BIND interface exhibits balanced polymer and metal phase, indicating bi-continuous pathway of both polymer and metal. More specifically, the balanced polymer and metal phases within the interpenetrating layer of BIND interface give a concentration of 75% and 25% for C and Au, respectively (FIG. 2K), with metal phase penetrating into BIND interface until a depth corresponding to ˜70 etching cycle (FIG. 2L). Such phase distribution influences the macroscopic electrical resistance. Although the metallic phase on the BIND interface is deeper than its non-adhesive counterpart, both interfaces have similar surficial and cross-sectional sheet resistance of <10 Ω/sq (FIG. 2M).

It has been demonstrated that the BIND connection, including mechanical and electrical pathways, originates from the interpenetrating polymer and metal phase at atomic level. Such connection does not come from the exposed polymer in the microcracks on BIND interface, which can be proved via reductio ad absurdum (FIG. 10A to 10L).

The reductio ad absurdum was carried out to prove that exposed polymer in microcrack is not the main reason of BIND connection.

The BIND interface, as microcrack-based stretchable electrode, has microcracks on the surface, while the opening and closing of microcrack endow its electrical stretchability. Although the substrate polymer, self-healing SEBS can be exposed via microcrack opening, it is not the main reason for the mechanical bonding in BIND connection. Reductio ad absurdum to was used to prove this.

Surface SEM on the non-conductive interface, BIND interface, and non-adhesive interface, respectively, was conducted under 0%, 50% and 100% strain (FIG. 10A to 10I). Here the Au thickness is 45 nm, and the evaporation rate determined the final surface properties. In images of non-conductive interface, the microcracks are very small and narrow, while the non-adhesive interface shows longer and wider microcracks. This is probably because the stacking of gold nanoparticles in non-adhesive interface is more brittle than the interpenetrating layer in non-conductive or BIND interface. According to the AFM mapping, the microcracks penetrate the interpenetrating layer in BIND interface, with a depth of ˜90 nm (FIG. 10J to 10L).

If the exposed polymer from microcracks is the main reason for BIND connection, then the non-adhesive interface should exhibit the strongest adhesion when pressed together. However, the electromechanical tests shows that the adhesion strength in fact decreases from non-conductive interface, to BIND interface, to non-adhesive interface, which is a completely opposite phenomenon. Therefore, the exposed polymer from microcracks is not the main reason for BIND connection.

Additionally, the biphasic interpenetration endows the BIND interface with high robustness to resist tape peeling (FIG. 2N, FIG. 3Q to 3S). The BIND interface remained intact after tape peeling, with nearly unchanged resistance. In comparison, the gold was easily peeled off in conventional PDMS/Au interface, leading to complete loss in conductivity.

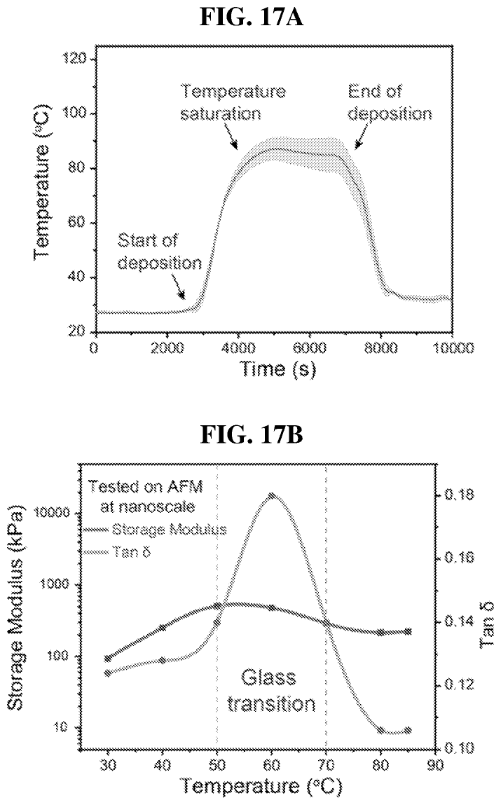

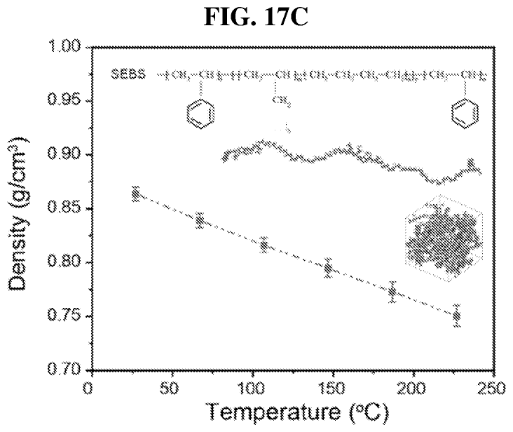

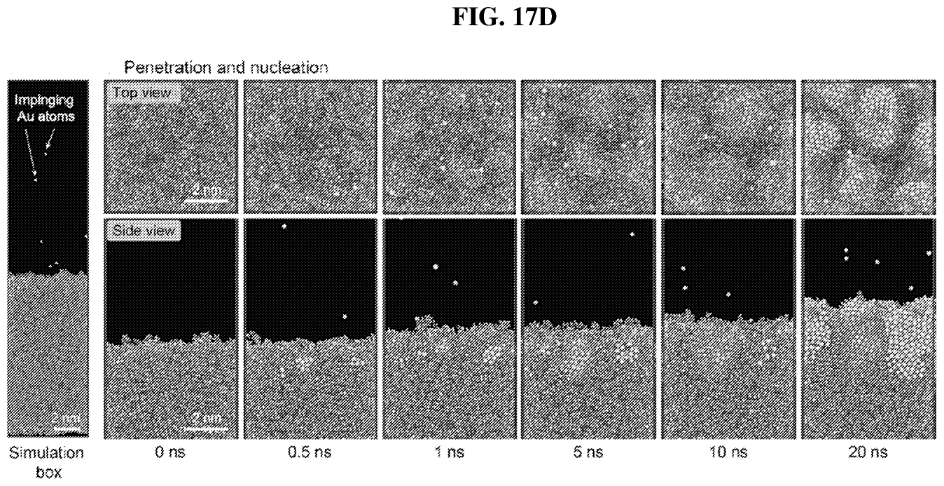

Furthermore, the nanomechanics process in the BIND interface formation was investigated, which can be divided into three stages: the initial stage, the nucleation stage, and the growth stage. This nanomechanics analysis is based on gas kinetics, experimental observations, and verified via molecular dynamics (MD) simulations, which clearly depicts the formation process of the biphasic, interpenetrating structure in BIND interface (FIG. 17A to 17D).

To investigate the nanomechanics in BIND interface formation, physical modelling and molecular dynamics (MD) simulations were conducted based on the Volmer-Weber or island growth theory, one of the three primary thin film deposition modes.

In the classic Volmer-Weber growth mode, the condensing atoms first nucleate into three-dimensional islands, followed by island growth and coalescence, resulting in continuous thin film on the substrate. Such growth mode occurs when the deposited thin film and substrate are composed of dissimilar materials without epitaxial relationship, so the film adatoms are more strongly coupled with each other than with the substrate. The deposition of Au thin film on SiO₂ and SiC was investigated extensively as examples of Volmer-Weber growth.

In this case, the thermal deposition of Au on block copolymer SEBS meets the condition of Volmer-Weber growth, yet replacement of originally inorganic crystalline substrate induces a new growth mechanism in terms of atom-molecular chain interaction, and new phenomenon in terms of bi-phasic nano-dispersed structure. Here the physical model of BIND interface formation during thermal deposition was constructed, which is a nanomechanics process of complete transformation of a flux of Au atoms from the gas phase into nano-dispersed structure, with three stages.

Stage I, initial stage: The initial physical picture of BIND interface formation was investigated quantitatively, providing thermodynamics parameters and material properties as inputs for MD simulations in next stage. For the Au atom flux, the heated Au source emits a gas phase atom flux under vacuum of 10⁻⁶ Torr. Following Maxwell-Boltzmann speed distribution, the root mean square velocity of Au atom vᵣₘₛ can be determined by the kinetic theory of gases: vᵣₘₛ=\sqrt{3RT/M}≈460 m/s, where T is the kelvin temperature, R the gas constant 8.314 J·K⁻¹·mol⁻¹, and M the molar mass of gas. Then, the Au atom flux density Jₙ can be calculated by the deposited Au on oscillating quartz crystal thickness sensor inside the thermal evaporator, assuming that Au on crystal sensor forms face centered cubic (fcc) packing, the most stable state of solid Au: Jₙ=s*D/V≈2.95*1018 (atom·s⁻¹·m⁻²), where s is the deposition rate calculated by crystal sensor, D is the fcc packing density (74%), and V is the Au atom volume.

For the resilient substrate, the mechanical properties of block copolymer SEBS in nanoscale were analysed at an elevated temperature. Such elevated temperature in SEBS copolymer during deposition comes from both thermal radiation from heated source underneath and the gold collision energy, where the latter consists of kinetic energy from Au atoms, cohesion energy from the transition of Au from vapor to solid, and interaction energy between Au atom and polymer. Experimentally, the substrate overall temperature was raised and saturated at around 90° C. during deposition, measured via a homemade in situ temperature sensor (FIG. 17A). Here the substrate experienced tensile stress and volume expansion. In this temperature range, the polymer viscoelasticity at nanoscale was investigated by AFM nanomechanical dynamic analysis (FIG. 17B). The tan 6, ratio of the loss to storage modulus, shows a peak in 50-70° C., corresponding to the glass transition associated with the hard polystyrene segments, indicating softening of polymer above the glass transition temperature.

Stage II, nucleation stage: In this stage, the flux of Au atoms impinges on the resilient polymersubstrate, penetrating the polymer surface and the nucleation starts. Here MD simulations based on the experimental/deduced parameters in stage I are employed to simulate this penetration and nucleation process.

Based on current computation capabilities in all-atom simulations, the atomistic molecular chain of the polymer is modelled with the similar styrene/ethylene-butylene ratio of ˜12:88, but at a reduced molecular weight ˜1,700 compared to ˜100,000 in experiments. Materials Studio was adopted to construct the amorphous block copolymer SEBS and the follow-up simulations were carried out based on LAMMPS using the COMPASS force field. The cut-off distance was set to 10 Å for the van der Waals and Coulomb interactions, where the electrostatic interactions are computed using the Particle-Particle-Particle-Mesh (PPPM) method with a precision of 10⁻⁶. All simulations were performed with a time step of 1 fs and under periodic boundary conditions (PBC). The NPT ensemble was adopted to simulate the bulk polymer systems at different temperatures (FIG. 17C). The simulated density was 0.86 g/cm³ at temperature of 27° C., close to the experimental value of 0.89 g/cm³, and the polymer density decreases with elevated temperature due to the increasing free volume, which result in less resistance to Au atom penetration.

To model the penetration and nucleation process of the Au atoms, the polymer substrate was constructed as a block with thickness around ˜12.6 nm inside a simulation box of 6.2×6.2×30 nm³ (FIG. 17D). The Au atoms were deposited from the top region with an initial velocity of 460 m/s toward the polymer substrate, following the calculation in stage I. Limited by the time scale of MD simulations, a relatively large deposition rate (˜250/ns) was chosen in the model to accelerate the whole deposition process. An artificial wall was introduced at the bottom to prevent the translation of the SEBS substrate. The Lennard-Jones potential compatible with COMPASS force field was adopted to describe the fcc Au metals and the interactions between Au atom and polymer. The Au atoms were modelled as an NVE ensemble while the substrate was modelled as an NVT ensemble with the experimental saturation temperature of 90° C. The time evolution of interface structures in MD simulations shows nucleation of Au atom flux in the early stage of deposition, where the incoming Au atoms initially penetrated into the substrate and then nucleated underneath the surface with depth of nanometres (FIG. 17D). As the subsequent Au atoms favourably bonded to the nuclei, both the nuclei size and penetration depth increase with time. Such penetration and nucleation arise uniquely from the influx of Au atom into polymer substrate composed of molecular chains with high mobility and low density based on experiments and simulations.

Stage III, growth stage: In this stage, the three-dimensional nuclei underneath the polymer surface undergoes continuous growth, penetration, and coalescence, finally forming the bi-phasic, interpenetrating nanostructure of the BIND interface. As more Au adatoms impinges the polymer surface over time, the as-formed nuclei in size of sub-nanometer gradually grow into larger nanoparticles. Experimentally it was observed that the final Au nanoparticles after deposition (that is, in BIND interface) have the size of −20 nm (FIG. 2a), containing thousands of Au atoms, and consistent with the nuclei-growth model. Besides the size, the penetration depth of Au nuclei/nanoparticles also increases over time, leading to the experimentally observed depth of −100 nm underneath polymer surface.

At the end of thermal deposition, the heated source was switched off with closed shutter, so substrate temperature was cooled down from 90° C. to room temperature. For the as-formed bi-phasic, interpenetrating nanostructure with −100 nm depth, its coefficient of thermal expansion (CTE) lies in between that of pure gold (˜15×10⁻⁶·K⁻¹) and pure SEBS (˜150×10⁻⁶·K⁻¹), largely different from the pure SEBS in further depth. This mismatch of thermal contraction leads to a compressive stress in the bi-phasic, interpenetrating nanostructure, which can be relieved either by buckling (FIG. 10A) or initial microcracks (FIGS. 10B and 10C), both beneficial to its electrical stretchability.

In all, the nanomechanics process in BIND interface formation was analyzed, based on the classic Volmer-Weber thin film growth theory. The three stages during formation process, the initial stage, the nucleation stage, and the growth stage, were analysed based on gas kinetics, experimental observations, and MD simulations, after which the bi-phasic, nano-dispersed nanostructure of BIND interface can be formed.

### Example 3C: Results and Discussion—BIND Device for In Vivo Neuromodulation

Employing BIND connection, various stretchable hybrid devices can be customized by assembling soft, rigid and encapsulation components together in personalized design. In vivo neuromodulation is an example requiring modular designed devices, where stretchable electrodes stimulate nerves and record evoked compound muscle action potential (CMAP). Here, different regions of the electrodes necessitate different properties: ultrathin electrodes are needed for contact with internal organ, by minimizing gap and increasing signal quality, while thick electrodes are required for robust wiring and anti-interference property. Therefore, the BIND electrode is composed of ultrathin, thick and encapsulation components via BIND connection (FIG. 3A, FIG. 4L to 4N). In its suture-free application, the BIND electrode was wrapped around nerve/muscles, and the ultrathin tail was pressed to form tight bonding due to its mechanical self-adhesion, before the supporting layer was removed (FIG. 3B). Compared to BIND electrode, one-piece electrode cannot fulfil both conformality and robustness: the all-ultrathin electrode crumpled easily in the wiring region, increasing its fragility to mechanical interference, while all-thick electrode cannot conformally wrap around tissue, leaving large gaps and resulted signal loss (FIG. 3C). For quantitative comparison, all the three types of electrodes are employed to detect subcutaneous CMAP signal (FIG. 3D). The representative signal from BIND electrode is also reliable, showing a consistent pattern of clear stimulation artifact and evoked CMAP signal (FIG. 3E). The lowest baseline noise and highest signal-to-noise ratio (SNR) of BIND electrodes show that such modular design that utilizes different form factors (thickness) can significantly enhance the signal quality (FIG. 3F).

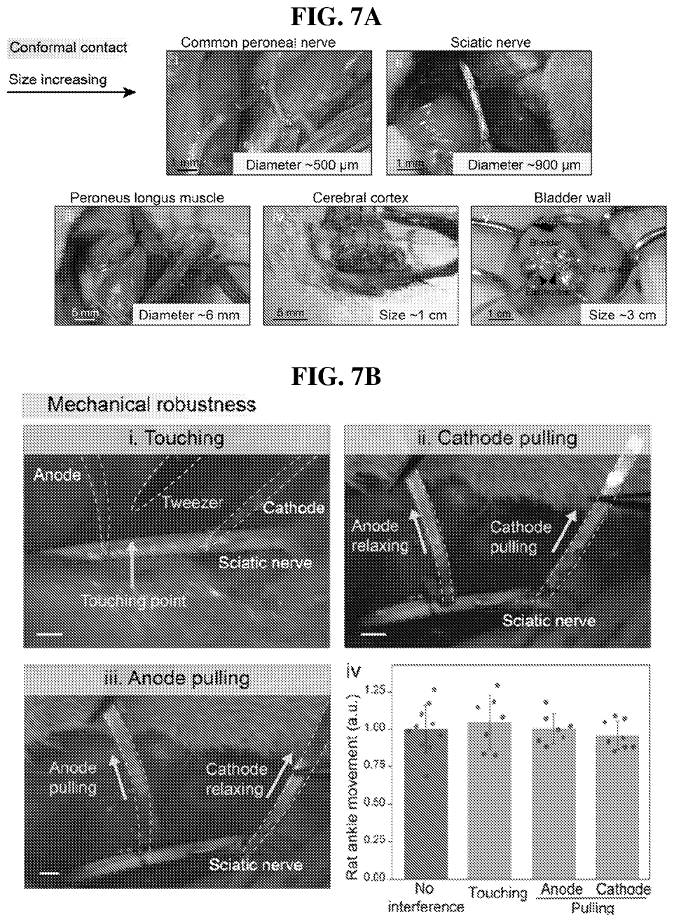

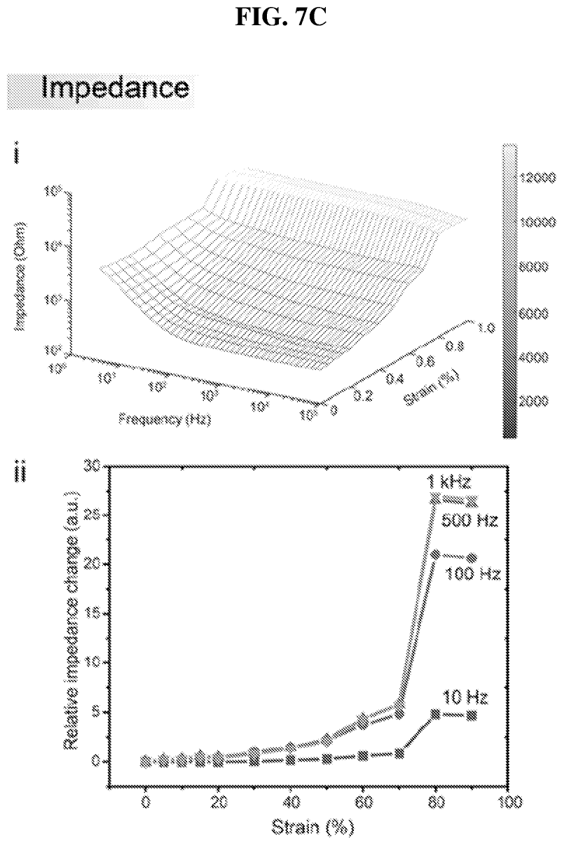

By this means, both conformal contact and robustness can be obtained via modular configuration of BIND electrodes. FIG. 7A to 7C also demonstrate for the BIND electrode, showing the robustness of in vivo neuromodulation BIND electrode, against mechanical interference. The BIND electrode, composed of the ultrathin part for conformal contact, and the thick part for robust wiring, was wrapped on sciatic nerve for stimulation. Here the thick wiring endows the robustness of the whole electrode, against mechanical interference. The evoked rat ankle movement, as result of sciatic nerve stimulation, shows little difference in case of nerve touching, cathode pulling, and anode pulling, demonstrating the resistance against mechanical interference. Conformal contact is evident on various internal tissues, with sizes ranging from cm to hundreds of μm, including sciatic nerve, common peroneal nerve, peroneus longus muscle, cerebral cortex and bladder wall (FIG. 7A). The proof for robustness can be seen from the resistance of the modular electrodes against mechanical interference commonly encountered in in vivo environment, such as touching, anode/cathode pulling, while still maintaining its electrical performance (FIG. 7B). Besides, the impedance of BIND interface is stable up to 70% strain, and the BIND connection only contributed to DC component in impedance (FIG. 7C).

To illustrate the feasibility and universality of BIND electrode, neuromodulation targeting different physiological functions was conducted, including subcutaneous CMAP, electrocorticography (ECoG) and bladder urination. For subcutaneous CMAP, both stimulation and 2-channel recording electrodes were constructed via BIND connection, and the recorded CMAP signal exhibits increasing and then saturated tendency via stimulation current (FIG. 3G, FIG. 8A to 8F). For ECoG recording, the 4-channel BIND electrode provided intimate contact with exposed soft cortex, resulting in prominent difference between healthy and epilepsy rat, in which the latter shows higher power in ECoG frequency range (FIG. 3H, FIG. 8G to 8H). For bladder stimulation, the BIND electrode was sutured on the bladder muscle wall, which transmitted electrical pulses to evoke bladder contraction and urination (FIG. 3I, FIG. 8I to 8K). The pressure changes via external sensor suggested successfully urination evoked from bladder wall stimulation. In all, the BIND electrode exhibited advantages in terms of conformal contact and robust wiring, compared to one-piece design, leading to high quality in vivo neuromodulation.

### Example 3D: Results and Discussion—BIND Device for In Vivo Neuromodulation

As a step further than the in vivo neuromodulation system, a more complex device—the 21-channel on-skin EMG electrode array—via BIND connection was synthesized, which utilized all three types of components (FIG. 4A). In this system, the 21-channel ultrathin, conformalelectrode with encapsulation provides conformal, airtight contact with skin, beneficial for high signal fidelity and spatial resolution. The collected signal was relayed to the thick and robust wiring with encapsulation, which acts as soft electrical wiring and mechanical transition region. Then the signal was memorized, transmitted, and processed by the customized PCB, based on Si-based microelectronics technology. These components with unique functions were fabricated separately, and then integrated via BIND connection, to put together a stretchable, 21-channel on-skin EMG electrode (FIG. 4B, FIG. 6L to 6O). As control, ACF-connected electrode with the same configuration was synthesized using conventional PDMS/Au electrodes and connected via commercial ACF.

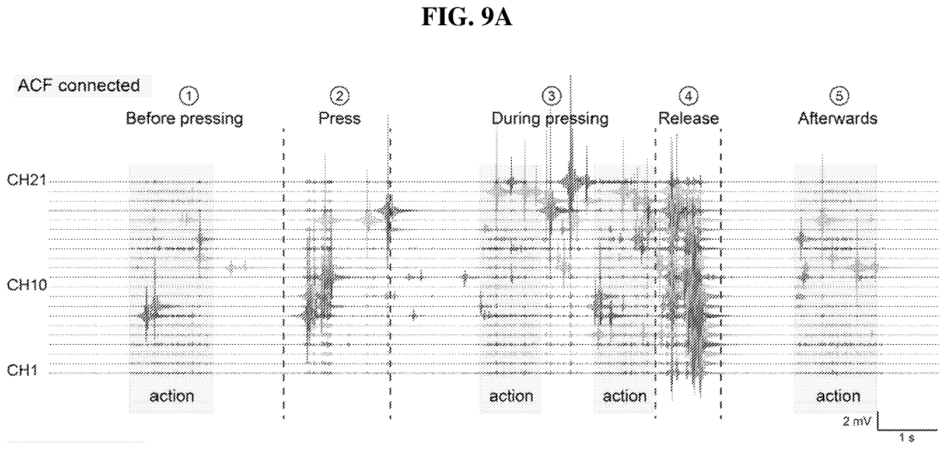

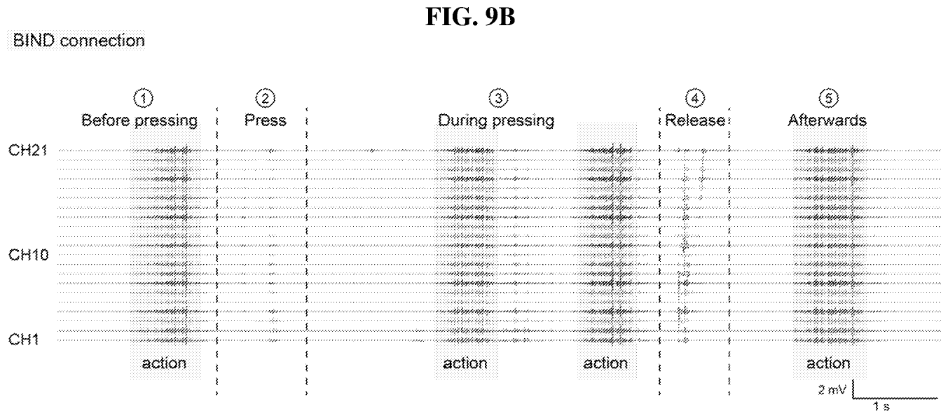

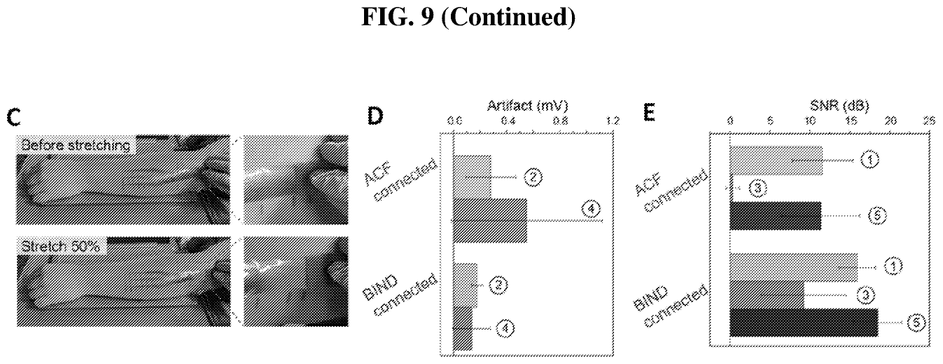

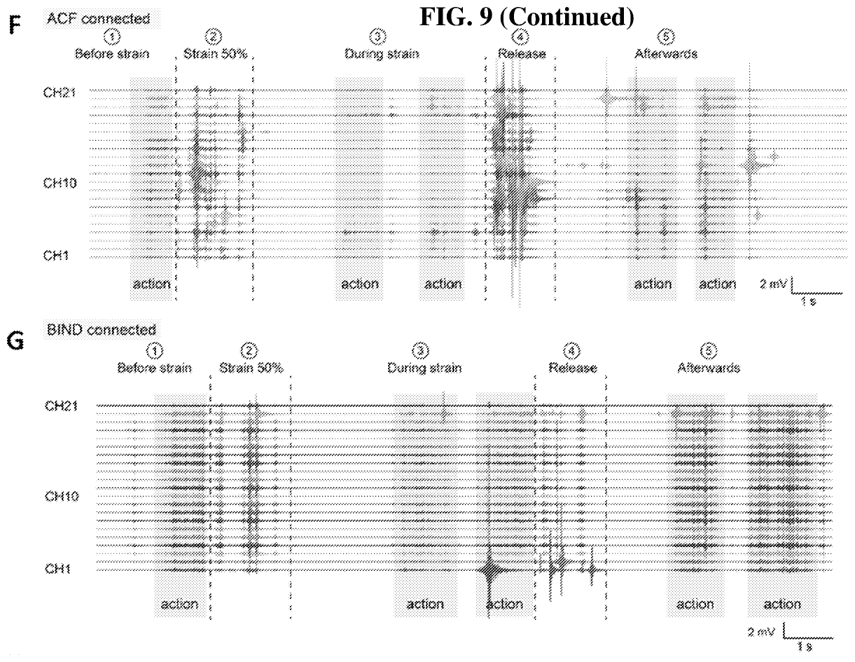

In a practical scenario, mechanical interference (pressure/strain) on connection is commonly encountered owing to strain concentration, which often leads to overwhelming noise or even electrode failure, resulting in deteriorated signal quality. Here it is shown that the present BIND electrode has a high resistance against such mechanical interference, due to the robustness of BIND connection. When connection pressure is applied (FIG. 4C), the BIND electrode shows clear EMG signal, in contrast with the weakened signal and large noise from ACF-connected control electrode (FIG. 4D, FIGS. 9A and 9B). In the BIND electrode, the pressing and releasing generated artifact of 0.03 mV and 0.12 mV, respectively, much lower than that of ACF-connected electrode (0.24 mV from pressing and 0.80 mV from releasing) (FIG. 4E). Meanwhile, in BIND electrode, the SNR of clenching action is 20.9 dB before pressing, 17.2 dB during pressing, and 20.3 dB afterwards, indicating high signal fidelity under pressure and good recovery ability. In contrast, the ACF-connected electrode with initial SNR of 11.0 dB suffers from significantly signal loss (−0.06 dB SNR) during pressing, and incomplete recovery afterwards (7.56 dB SNR) (FIG. 4F). Except for pressure, the BIND electrode also exhibited resistance against connection strain up to 50% (FIG. 9C to 9G). Its low stretching/releasing artifact (0.18 and 0.13 mV) and high SNR (9.2 dB during stretching and 18.4 dB afterwards) manifest its superiority to ACF-connected electrode, which has stretching/releasing artifact of 0.28 and 0.55 mV, and low SNR during and after stretching (0.32 dB and 11.34 dB).

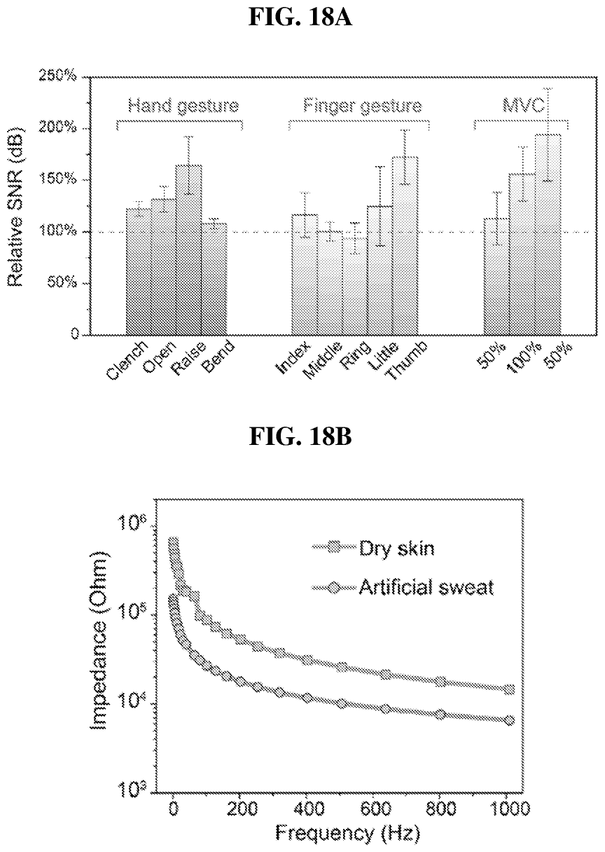

Employing the BIND electrode, 21-channel EMG mapping can be obtained from various gestures, including hand movement (clench, open, raise, bend), finger movement (stretching of individual fingers) and different levels of maximum voluntary contraction (MVC) (FIG. 4G, FIG. 10M). Moreover, the BIND electrode can work underwater, while maintaining its detection capability and even the resistance against mechanical interference (FIG. 4H, FIG. 10N) as well as on sweaty skin after exercise (see FIGS. 18A and 18B). This proved the airtightness of the BIND connection itself, as well as the contact between ultrathin, conformal electrode and skin.

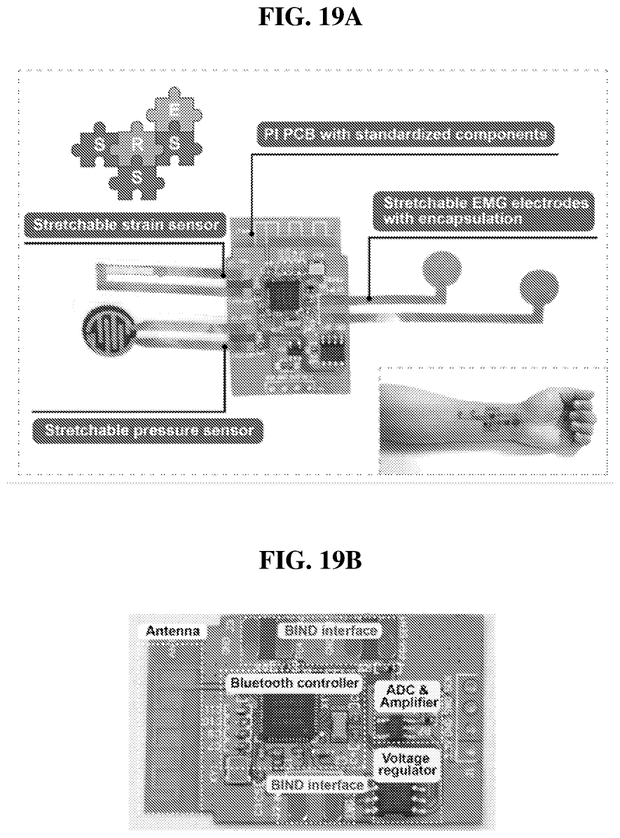

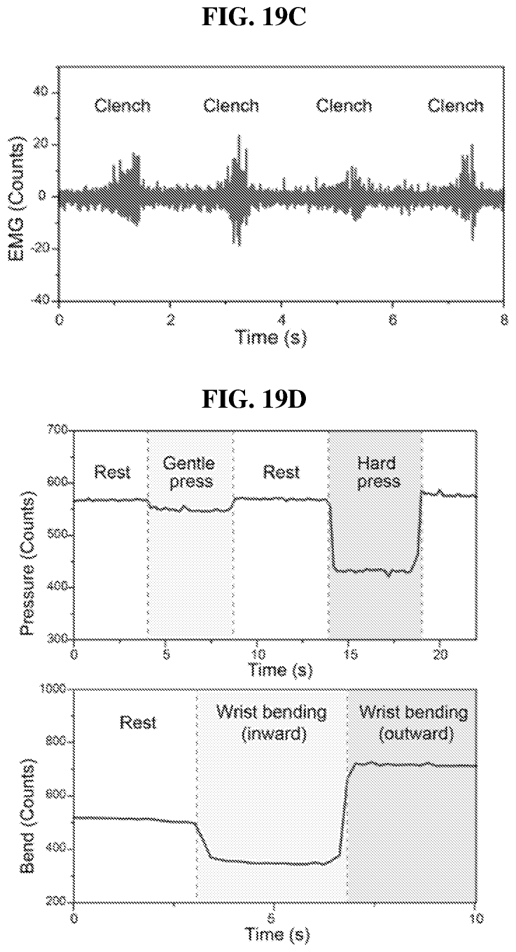

Therefore, given its high spatial resolution, resistance against mechanical interference, and waterproof capability, the 21-channel on-skin BIND electrode can provide high-quality EMG data for health monitoring and diagnostics. The efficiency and accuracy of gesture reconstruction can be further improved, if the BIND electrode provides input for more advanced algorithm. In addition, using such plug-and-play BIND connection, simultaneous detection of EMG together with pressure and strain signals can also be achieved with other customized circuit design (see FIG. 19A to 19D). These experiments demonstrate that BIND connections can assemble various modules into complex stretchable devices in a plug-and-play way, where its high-quality signals benefit health monitoring and diagnostics.

### Example 3E: Summary of Examples 1 to 3D

The above examples demonstrated a highly stretchable BIND connection to robustly integrate soft, rigid and encapsulation components together, into stretchable hybrid electronic devices. Such BIND connection is composed of a BIND interface, where interpenetrating polymer and metal phases constructed continuous mechanical and electrical pathway. For soft-soft BIND connection, high electrical (>180%) and mechanical (>600%) stretchability was realized. Its paste-free feature endows high resolution patterning and conformality for ultrathin design. Besides, its multiple metallic material choice and anti-tearing expands the practical application range. For soft-rigid BIND connection, high stretchability of ˜200% was also achieved within diverse rigid materials (PI, PET, glass). For soft-encapsulation BIND connection, the peeling force of 22 times larger than conventional encapsulation exhibits high adhesion. As typical stretchable hybrid device, in vivo neuromodulation electrode was integrated via BIND connection, which performed better than one-piece electrode due to its conformal contact and robust wiring. Further, a more complicated hybrid device, the 21-channel on-skin EMG electrode was integrated via BIND connection, exhibiting high resistance against mechanical interference, as well as airtightness in underwater environment. Employing the present BIND interface, more potential could be tapped to integrate stretchable hybrid device with various functionalities and complexity, especially for on-skin/implantable human-machine interface.

### Example 4A: Fabrication of Present Device for Use as an Interconnect

The present device may be usable and/or incorporable into another device as an interconnect. As the present device operable as an interconnect is binder-free and stretchable, it can be referred to herein as a binder-free, stretchable interconnect. The interconnect can integrate different stretchable modules together, and maintain its electrical and mechanical integration under mechanical strain.

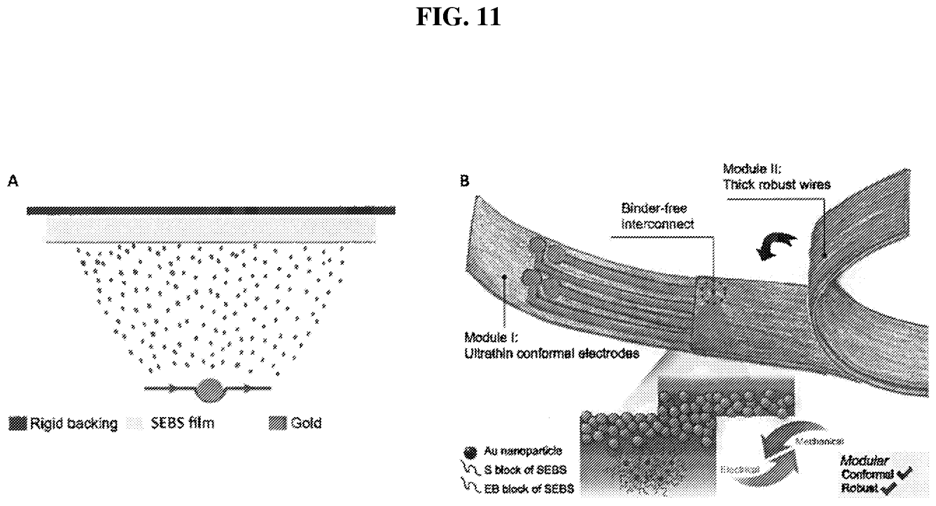

In one-limiting example, a thermoplastic elastomer of sytrene ethylene butylene styrene (SEBS) was selected as the stretchable substrate for forming a single stretchable electrode due to its self-healing properties (i.e. when the materials contact each other, they can adhere to each other. This is due to a process of interfacial partial polymer welding. SEBS is a non-limiting example of one such self-healing polymer). The SEBS solution in toluene (15 wt %) was poured into customized mold inside fume hood, and evaporate in at room temperature for 2 days, to get a transparent, stretchable thin film, with thickness of ˜100 μm. Then thick gold nanoparticle network was integrated on the surface of the SEBS polymer substrate via thermal deposition to form an electrically conductive pathway (FIG. 11A). The evaporation parameters were configured to render thickness of 45, 60, 75, and 90 nm, and evaporation rate of 0.1, 0.2, 0.5, 1.0, 2.0, 5.0, 10.0 Å/s. This parameter set is configurable to render the desired properties of the interconnect. The as-prepared single electrode comprise the gold nanoparticle network on top of stretchable elastomer, and it maintains electrical conductivity under mechanical strain. Then one electrode was placed upside down on top of another, with a small overlap area (FIG. 1B). By gently finger pressing of ˜10 s, the two electrodes were strongly adhered to each other. Such bonding includes both electrical bonding and mechanical bonding, which means the two single electrodes are integrated together functionally through the interconnect.

### Example 4B: Characterization of the Interconnect of Example 4A

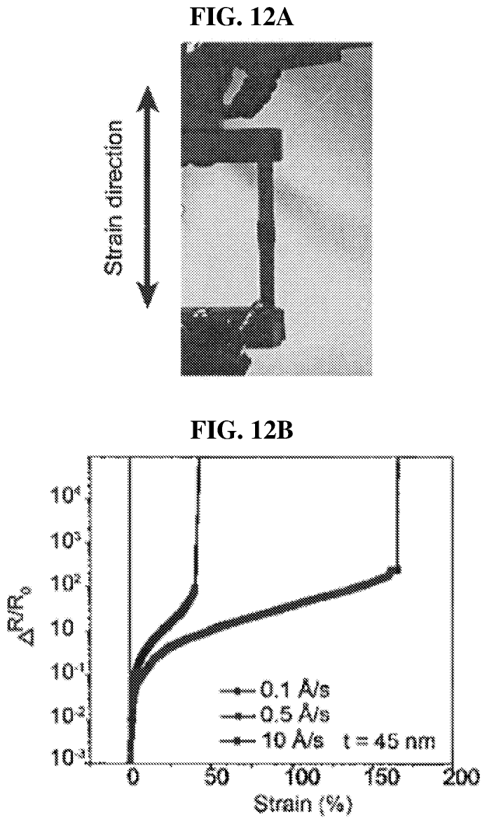

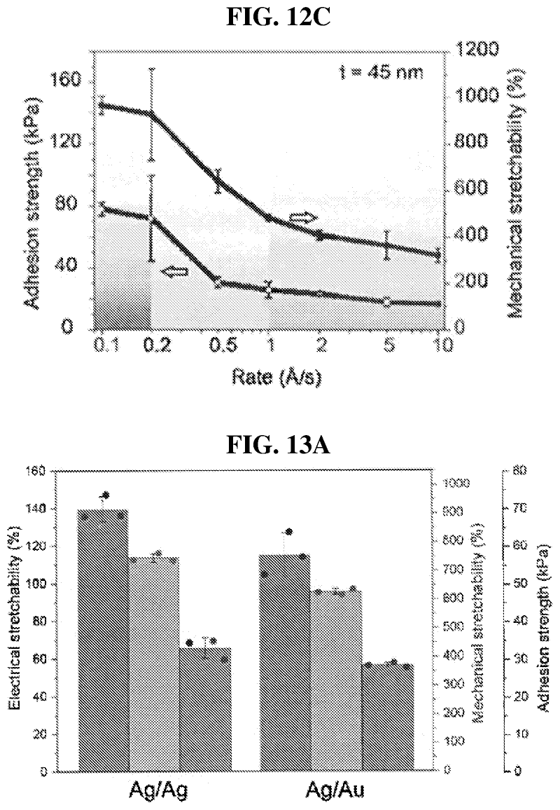

Mechanical and electromechanical test was employed to test the mechanical and electrical properties of the stretchable interconnect, respectively. Here the area of interconnect was 3.5*0.5 cm², with an overlapping area of 0.5*0.5 cm². The interconnect was clamped on grips of MTS M43 mechanical tensile machine, and stretcha until it was mechanically broken (FIG. 12A). The electrical resistance change, and the mechanical load during mechanical stretching was recorded. For thickness of 45 nm gold, interconnects with different evaporation rate shows different ability of electrical conductivity maintenance (FIG. 12B). The interconnect with 0.5 Å/s evaporation rate has electrical sheet resistance of ˜10 ohm/sq before stretching, and maintains conductivity (which is electron pathway) until ˜160% tensile strain. It was mechanically broken after ˜750% strain, which means it can endure large mechanical stretching (FIG. 12C).

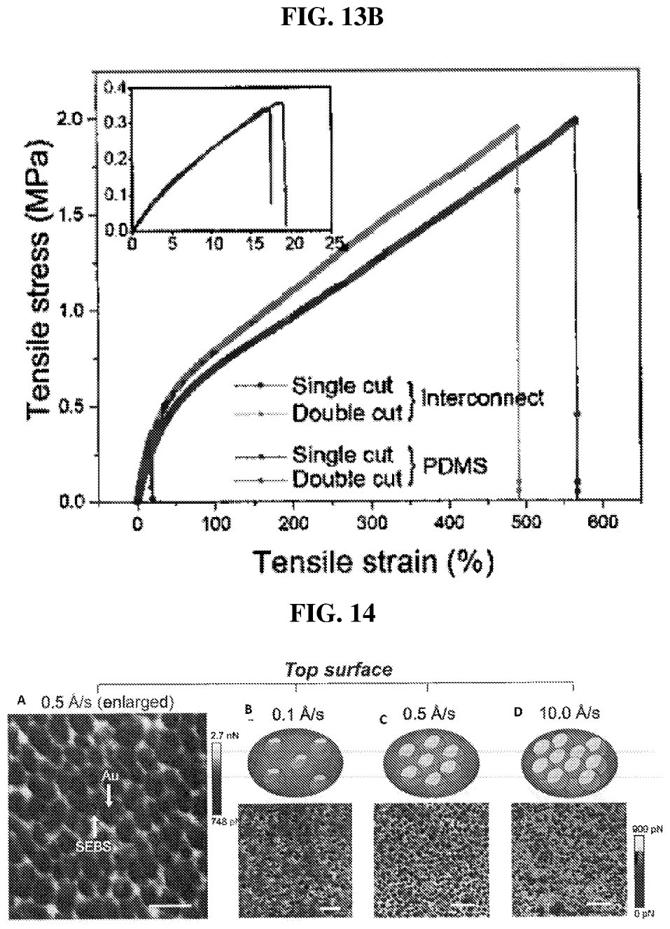

To investigate the universality of this interconnect, silver was employed to replace the gold nanoparticle network for characterization. Both silver and gold are widely applied in field of stretchable electronics, because silver has high electrical and thermal conductivity, cost evaluation while gold has high electrical conductivity and unblemished property. So, it is one material suitable for integrating different stretchable modules together which was made of silver and/or gold. The mechanical and electromechanical results show that, other than Au/Au, the present interconnect can be formed within Au/Ag and Ag/Ag (FIG. 13A). The mechanical stretchability, electrical stretchability and adhesion strength exhibit little difference in between silver and gold. Therefore, it has great potential in stretchable electronics due to such material compatibility.

Moreover, the present interconnect, when configured in electrodes, is able to render the electrodes endurable against mechanical damage without easily breaking. For polydimethylsiloxane (PDMS), if there is mechanical damage (e.g. a notch) on the thin film, it can easily break even under small strain. Since the practical application environment may induce mechanical damage into the device, intentionally or unintentionally, a stretchable interconnect with notch-insensitive property is needed. In the present stretchable interconnect based on SEBS, a blade was used to make one or two cuts (0.2 mm width) in the middle, and stretch the interconnect to gauge if it breaks (FIG. 13B). The mechanical test result shows that the interconnect with one or two cut can still maintain its integrity under more than 500% strain. In comparison, the cut PDMS sample easily breaks at 20% strain. Such notch-insensitive property of the present interconnect and device is very advantageous in practical applications and real environment.

### Example 4C: Study and Discussion of the Interconnect of Example 4A

To investigate the underlying mechanism of the interconnect, an adhesion mapping on the surface of one of the electrodes, via atomic surface microscopy (AFM), was carried out. Since the supramolecule SEBS polymer has much larger adhesion to AFM tip than the Au nanoparticles, the phases of SEBS and Au can be easily distinguished via adhesion mapping (FIG. 14A) clearly showing the co-existence of both SEBS and Au on the surface of the electrode. When two single electrodes overlap face-to-face, the SEBS polymer adhere to each other due to its self-healing property, resulting in mechanical binding, and the Au nanoparticles form ohmic contact with each other, resulting in electrical binding. Therefore, the as-formed interconnect has both mechanical and electrical stretchabilty. For different fabrication parameters, the ratio of SEBS polymer and Au nanoparticles on the surface is different. For single electrode with evaporation rate of 0.1 Å/s, the gold network immersed deeper inside the SEBS polymer, which exposed more polymer for stronger mechanical bonding (FIG. 14B). For evaporation rate of 0.5 Å/s, the ratio of Au and SEBS polymer reaches a balance (FIG. 14C). For evaporation rate of 10 Å/s, the gold nanoparticles network was piled on top of SEBS polymer rendering stronger electrical bonding but weaker mechanical bonding (FIG. 14D). these results are consistent with the electromechanical results. It also shows the performance of the present interconnect can be easily configured via its fabrication parameters.

### Example 5: Commercial and Potential Applications

The present flexible electronic device having the binder-free and stretchable interconnect is incorporable and/or combinable with other devices to form a stretchable electronic system. There is no limitation to the functions of other devices even when such an interconnect is used. Therefore, any kind of stretchable devices can be compatibly used, including physical, chemical, physiological sensors, memory, synapses, energy storage devices, etc. Potential and commercial applications of the present flexilbe electronic device include, without being limited to, wearable healthcare devices, implantable eletronics, intraoperative tools, soft robotics, etc.

While the present disclosure has been particularly shown and described with reference to specific embodiments, it should be understood by those skilled in the art that various changes in form and detail may be made therein without departing from the spirit and scope of the present disclosure as defined by the appended claims. The scope of the present disclosure is thus indicated by the appended claims and all changes which come within the meaning and range of equivalency of the claims are therefore intended to be embraced.

1. A flexible electronic device comprising:
a first component comprising a first biphasic portion; and
a second component,
wherein the first component and the second component are in contact with an electrically conductive stretchable interface configured between the first component and the second component,
wherein the electrically conductive stretchable interface comprises the first biphasic portion which is adhered to a portion of the second component, and
wherein the first biphasic portion comprises a first polymer having
(i) a surface partially covered with metal nanoparticles which are partially exposed at the surface, and
(ii) metal nanoparticles which are completely embedded in the first polymer.

2. The flexible electronic device of claim 1, wherein the electrically conductive stretchable interface is absent of an adhesive paste.

3. The flexible electronic device of claim 1, wherein the portion of the second component adhered to the first biphasic portion comprises a second biphasic portion.

4. The flexible electronic device of claim 3, wherein the second biphasic portion comprises a second polymer having
(i) a surface partially covered with metal nanoparticles which are partially exposed at the surface, and
(ii) metal nanoparticles which are completely embedded in the second polymer,
wherein the metal nanoparticles of the first biphasic portion and the second biphasic portion, which are partially exposed, are in contact.

5. The flexible electronic device of claim 4, wherein the first polymer comprises styrene ethylene butylene styrene or styrene-butadiene, and wherein the second polymer comprises styrene ethylene butylene styrene or styrene-butadiene.

6.-7. (canceled)

8. The flexible electronic device of claim 1,
wherein the first polymer and the metal nanoparticles residing (i) proximal to the surface of the first polymer and (ii) at a depth of up to 10 nm from the surface of the first polymer, have a weight ratio of 40:60 to 60:40; or
wherein the first polymer and the metal nanoparticles residing (i) proximal to the surface of the first polymer and (ii) at a depth of up to 10 nm from the surface of the first polymer have a weight ratio of 40:60 to 60:40, and wherein the first polymer and metal nanoparticles residing at a depth of more than 10 nm and up to 100 nm from the surface of the first polymer have a weight ratio of 30:70 to 70:30.

9. The flexible electronic device of claim 4,
wherein the second polymer and the metal nanoparticles residing (i) proximal to the surface of the second polymer and (ii) at a depth of up to 10 nm from the surface of the second polymer, have a weight ratio of 40:60 to 60:40; or
wherein the second polymer and the metal nanoparticles residing (i) proximal to the surface of the second polymer and (ii) at a depth of up to 10 nm from the surface of the second polymer have a weight ratio of 40:60 to 60:40, and wherein the second polymer and metal nanoparticles residing at a depth of more than 10 nm and up to 100 nm from the surface of the second polymer have a weight ratio of 30:70 to 70:30.

10. The flexible electronic device of claim 4, wherein the metal nanoparticles, which are completely embedded in the first polymer and the second polymer, are present in the first polymer and the second polymer up to a depth of 90 nm.

11. The flexible electronic device of claim 1, wherein:
the first component and the second component have identical rigidity; or
the first component has a higher rigidity than the second component.

12. The flexible electronic device of claim 1, wherein the second component is an encapsulation layer.

13. The flexible electronic device of claim 11, wherein the first component having the higher rigidity than the second component comprises polyimide, polyethylene terephthalate, glass, or silicon.

14. The flexible electronic device of claim 1, wherein the flexible electronic device is an electrode or an interconnect, and wherein the electrode is:
a neuro-modulation electrode, or
a 21-channel electromyography electrode attachable to a surface of a skin.

15. (canceled)

16. A method of forming the flexible electronic device of claim 1, comprising:
forming the first component comprising the first biphasic portion;
forming the second component; and
pressing the first component and the second component against each other to form the electrically conductive stretchable interface configured between and in contact with both the first component and the second component,
wherein the electrically conductive stretchable interface comprises the first biphasic portion which is adhered to a portion of the second component,
wherein the first biphasic portion comprises the first polymer having
(i) a surface partially covered with metal nanoparticles which are partially exposed at the surface, and
(ii) metal nanoparticles which are completely embedded in the first polymer.

17. The method of claim 16, wherein forming the first component comprises:
arranging the first polymer to face a metal source; and
heating the metal source to evaporate metal from the metal source to have metal nanoparticles incorporated to the first polymer, thereby rendering the first biphasic portion.

18. The method of claim 16, wherein forming the second component comprises:
arranging a second polymer to face a metal source; and
heating the metal source to evaporate metal from the metal source to have metal nanoparticles incorporated to the second polymer, thereby rendering a second biphasic portion in the portion of the second component.

19. The method of claim 16, when the first component has a higher rigidity than the second component, forming the first component comprises:
providing a rigid substrate;
treating the rigid substrate with oxygen plasma prior to contacting the rigid substrate with an organosilane;
forming the first polymer on the rigid substrate;
arranging the rigid substrate to have the first polymer face a metal source; and
heating the metal source to evaporate metal from the metal source to have metal nanoparticles incorporated to the first polymer, thereby rendering the first biphasic portion.

20. The method of claim 16, when the first component has a higher rigidity than the second component, forming the second component comprises:
arranging a second polymer to face a metal source; and
heating the metal source to evaporate metal from the metal source to have metal nanoparticles incorporated to the second polymer, thereby rendering a second biphasic portion in the portion of the second component.

21. The method of claim 16, when the second component is an encapsulation layer, forming the first component comprises:
arranging the first polymer to face a metal source; and
heating the metal source to evaporate metal from the metal source to have metal nanoparticles incorporated to the first polymer, thereby rendering the first biphasic portion.

22. The method of claim 16, when the second component is an encapsulation layer, forming the second component comprises:
providing an encapsulation material as the second component;
or
providing a substrate;
treating the substrate with oxygen plasma prior to contacting the substrate with an organosilane; and
depositing an encapsulation material on the substrate to form the encapsulation layer.

23. The method of claim 16, wherein the flexible electronic device is a neuro-modulation electrode or a 21-channel electromyography electrode,
wherein forming the first component comprises:
providing a rigid substrate;
treating the rigid substrate with oxygen plasma prior to contacting the rigid substrate with an organosilane;
forming the first polymer on the rigid substrate;
arranging the rigid substrate to have the first polymer face a metal source;
heating the metal source in the presence of a mask to evaporate metal from the metal source to have metal nanoparticles incorporated to the first polymer according to a pattern defined by the mask, thereby rendering the first biphasic portion; and
depositing an encapsulation material on the first polymer in a manner which exposes the first biphasic portion; and
wherein forming the second component comprises:
arranging a second polymer to face a metal source;
heating the metal source to evaporate metal from the meta source to form metal lines on the second polymer; and
depositing an encapsulation material to cover the metal lines except for two opposing ends of each of the metal lines.

24. (canceled)
